(19) United States
(12) Patent Application Publication
(10) Pub. No.: US 2026/0314550 A1
(43) Pub. Date: Oct. 15, 2026

(54) GENETIC MARKERS ASSOCIATED WITH INCREASED FERTILITY IN MAIZE

(71) Applicant: SYNGENTA CROP PROTECTION AG, BASEL (CH)

(72) Inventors: Zhigang GUO, Research Triangle Park, NC (US); Mary DUNN, San Diego, CA (US); Donna Ellen DELANEY, Slater, IA (US); Maria Elena FARICELLI, Bronxville, NY (US); Jonathan COHN, Research Triangle Park, NC (US); Ruijuan TAN, Research Triangle Park, NC (US)

(73) Assignee: SYNGENTA CROP PROTECTION AG, BASEL (CH)

(21) Appl. No.: 19/482,881

(22) PCT Filed: May 7, 2024
(86) PCT No.: PCT/US2024/028116

§ 371 Date: Nov. 10, 2025

Related U.S. Application Data
(60) Provisional application No. 63/501,094, filed on May 9, 2023.

(51) Int. Cl. A01H 1/04; A01H 1/02; C12Q 1/6895

(52) U.S. Cl. CPC A01H 1/045; A01H 1/022; C12Q 1/6895; C12Q 2600/13; C12Q 2600/156

(57) ABSTRACT

Compositions and methods to increased fertility of MIR162 plants are provided. Compositions include maize plants comprising fertile QTLs and/or transposable elements. Methods and kits are provided for identifying and producing these plants via transgenic means, breeding or genomic editing approaches.

### RELATED APPLICATION

This application claims priority to U.S. Provisional Application No. 63/501,094, filed on May 9, 2023. The entire content of said provisional application is herein incorporated by reference for all purposes.

### FIELD

This disclosure relates to the field of plant biotechnology.

### SEQUENCE LISTING

The official copy of the sequence listing is submitted electronically as an XML formatted sequence listing with a file named 109770_82681_SL, created on Apr. 23, 2024, and having a size of 304,751 bytes, and is filed concurrently with the specification. The sequence listing contained in this xml formatted document is part of the specification and is herein incorporated by reference in its entirety.

### BACKGROUND

The MIR162 event is a commercially important event for controlling certain pests in maize, such as fall armyworm. However, the MIR162 locus has been observed to be associated with decreased male fertility in certain inbred maize genetic backgrounds. The degree to which male fertility is impacted is inbred specific: some inbreds exhibit little or no reduction in male fertility when homozygous for the MIR162 locus; and other inbreds exhibit a significant reduction in male fertility when homozygous for the MIR162 locus. The degree to which male fertility is decreased is also affected by environmental factors, such as water availability and temperature. Furthermore, drought and high temperature conditions can increase MIR162-associated reductions in male fertility, which can make seed production more challenging and costly. There remains a need for methods to identify and use genetic backgrounds that are more likely to be associated with increased male fertility in the presence of the MIR162 locus.

### BRIEF SUMMARY

In one aspect, provided herein is a method of producing an MIR162 plant with increased fertility, said method comprising: selecting a first maize plant comprising one or more fertile QTLs, wherein the one or more fertile QTLs comprise one or more marker loci of SEQ ID NOs: 1-223 or 255, wherein at least one marker locus of the one or more marker loci comprises a favorable allele at position number 100 as shown in Table 3 or Table 4 or Table 9, crossing the first maize plant with a second maize plant to produce a plurality of progeny plants, wherein the first maize plant and/or the second maize plant comprises maize event MIR162, and selecting from the plurality of progeny plants a selected progeny MIR162 plant comprising one or more fertile QTLs.

In another aspect, provided herein is a method of producing a MIR162 plant with increased fertility, said method comprising: selecting a first maize plant comprising one or more marker loci linked to and within 10 centimorgans (cM) of one or more fertile QTL, wherein the one or more fertile QTLs are selected from the group consisting of a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3; a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4; a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4; a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4; a QTL located on chromosome 8 at position 14,787,385 comprising the marker locus of SEQ ID NO: 255, wherein the marker locus comprises a favorable allele at position number 100 as shown in Table 9; and a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4, and crossing the first maize plant with a second maize plant to produce a plurality of progeny plants, wherein the first maize plant and/or the second maize plant comprises maize MIR162 event; and selecting from the plurality of progeny plants a selected progeny MIR162 plant comprising the one or more fertile QTL using the one or more marker loci linked to and within 10 centimorgans (cM) of the one or more fertile QTL.

In yet another aspect, provided herein is a method of producing a MIR162 plant with increased fertility, said method comprising: selecting a first maize plant comprising one or more fertile QTLs, wherein the one or more fertile QTLs comprise one or more marker loci of SEQ ID NOs: 1-223 or 255, wherein at least one marker locus of the one or more marker loci comprises a favorable allele at position number 100 as shown in Table 3 or Table 4 or Table 9, and introducing a MIR162 event into the selected maize plant.

In yet another aspect, provided herein is a method of producing a MIR162 plant with increased fertility, said method comprising: selecting a first maize plant comprising one or more marker loci linked to and within 10 centimorgans (cM) of one or more fertile QTL, wherein the one or more fertile QTLs are selected from the group consisting of a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3; a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4; a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4; a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4; a QTL located on chromosome 8 at position 14,787,385 comprising the marker locus of SEQ ID NO: 255, wherein the marker locus comprises a favorable allele at position number 100 as shown in Table 9; and a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4, and introducing a MIR162 event into the selected maize plant.

In yet another aspect, provided herein is a method of producing a MIR162 plant with increased fertility, said method comprising: selecting a first maize plant comprising a transposon element (TE) comprising SEQ ID NO: 232, 233, or a variant thereof that is at least 90% identical to SEQ ID NO: 232 or 233, crossing the first maize plant with a second maize plant to produce a plurality of progeny plants, wherein the first maize plant and/or the second maize plant comprises maize MIR162 event, and selecting from the plurality of progeny plants a selected progeny MIR162 plant comprising the transposon element.

In yet another aspect, provided herein is a method of producing a plant compatible with a MIR162 event, said method comprising: selecting a first maize plant comprising a transposon element (TE) comprising SEQ ID NO: 232, 233, or a variant thereof that is at least 90% identical to SEQ ID NO: 232 or 233, crossing the first maize plant with a second maize plant to produce a plurality of progeny plants, and selecting from the plurality of progeny plants a selected progeny plant comprising the transposon element.

In yet another aspect, provided herein is a MIR162 plant comprising one or more fertile QTLs, wherein the one or more fertile QTLs comprise one or more marker loci of SEQ ID NOs: 1-223 or 255, wherein at least one marker locus of the one or more marker loci comprises a favorable allele at position number 100 as shown in Table 3 or Table 4 or Table 9.

In yet another aspect, provided herein is a MIR162 plant comprising one or more marker loci linked to and within 10 centimorgans (cM) of one or more fertile QTL, wherein the one or more fertile QTLs are selected from the group consisting of a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3; a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4; a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4; a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4; a QTL located on chromosome 8 at position 14,787,385 comprising the marker locus of SEQ ID NO: 255, wherein the marker locus comprises a favorable allele at position number 100 as shown in Table 9; and a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4.

In yet another aspect, provided herein is a MIR162 plant comprising one or more marker loci linked to and within 10 centimorgans (cM) of one or more fertile QTL, wherein the one or more fertile QTLs are selected from the group consisting of a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3; a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4; a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4; a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4; a QTL located on chromosome 8 at position 14,787,385 comprising the marker locus of SEQ ID NO: 255, wherein the marker locus comprises a favorable allele at position number 100 as shown in Table 9; and a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4.

In yet another aspect, provided herein is a kit for genotyping a plant wherein the kit comprises reagents for detecting the presence of one or more marker loci of SEQ ID Nos: 1-223 or 255; one or more marker loci linked to and within 10 centimorgans (cM) of one or more fertile QTL, wherein the one or more fertile QTLs are selected from the group consisting of a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3; a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4; a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4; a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4; a QTL located on chromosome 8 at position 14,787,385 comprising the marker locus of SEQ ID NO: 255, wherein the marker locus comprises a favorable allele at position number 100 as shown in Table 9; and a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4; and/or a transposon element (TE) of SEQ ID NO: 232, 233, or a variant thereof that is at least 90% identical to SEQ ID NO: 232 or 233.

In yet another aspect, provided herein is a method of detecting one or more marker loci linked to and within 10 centimorgans (cM) of one or more fertile QTL, the method comprising genotyping one or more fertile QTLs from the group consisting of a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3; a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4; a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4; a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4; a QTL located on chromosome 8 at position 14,787,385 comprising the marker locus of SEQ ID NO: 255, wherein the marker locus comprises a favorable allele at position number 100 as shown in Table 9; and a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4.

In yet another aspect, provided herein is a method of detecting one or more fertile QTLs in an MIR162 plant, the method comprising genotyping the plant at one or more marker loci linked to one or more fertile QTL, wherein the fertile QTL comprises one or more marker loci of SEQ ID Nos 1-223 or 255, wherein at least one marker locus of the one or more marker loci comprises a favorable allele at position number 100 as shown in Table 3 or Table 4 or Table 9.

In yet another aspect, provided herein is a method of detecting one or more marker loci linked to and within 10 centimorgans (cM) of one or more fertile QTL, the method comprising genotyping one or more fertile QTLs from the group consisting of a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3; a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4; a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4; a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4; a QTL located on chromosome 8 at position 14,787,385 comprising the marker locus of SEQ ID NO: 255, wherein the marker locus comprises a favorable allele at position number 100 as shown in Table 9; and a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4.

### DETAILED DESCRIPTION

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

Provided herein are methods and compositions related to identifying genomic loci associated with increased or decreased fertility in maize plants comprising MIR162 event.

These loci can be used to select plants having genetic backgrounds that have a higher likelihood of a successful MIR162 event conversion. Plants having genomic loci identified as associated with increased fertility can be selected and used in a breeding program to produce maize MIR162 plants having increased fertility as compared to control plants. Conversely, plants having genomic loci identified as associated with decreased fertility can be excluded from such a breeding program. These loci can so be used to rescue the conversion failure phenotype by introducing a favorable allele at a genomic locus (e.g., by breeding or genome editing) into a genetic background that contains the unfavorable allele at the genomic locus.

### I. Terminology

A “coding sequence” is a nucleic acid sequence that is transcribed into RNA such as mRNA, rRNA, tRNA, snRNA, sense RNA or antisense RNA. Preferably the RNA is then translated in an organism to produce a protein.

As used herein, “locus” is a chromosome region or chromosomal region where a polymorphic nucleic acid, trait determinant, gene, or marker is located. A locus can represent a single nucleotide, a few nucleotides or a large number of nucleotides in a genomic region.

The loci of this disclosure comprise one or more polymorphisms in a population (e.g., alternative alleles are present in some individuals).

As used herein, the term “allele” refers to one of two or more different nucleotides or nucleotide sequences (or the absence thereof) that occur at a specific locus or set of contiguous loci. In some embodiments, the term “allele” may be used interchangeably with the term “marker.”

As used herein, a “centimorgan” (cM) is a unit of measure of recombination frequency and genetic distance between two loci. One cM is equal to a 1% chance that a marker at one genetic locus will be separated from a marker at a second locus due to crossing over in a single generation. As used herein, “closely linked” means that the marker or locus is within about 20 cM, 15 cM, 10 cM, 5 cM, 41 cM, 3 cM, 2 cM, 1 cM, 0.5 cM, or less than 0.5 cM of another marker or locus. For example, 20 cM means that recombination occurs between the marker and the locus with a frequency of equal to or less than about 20%.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny. The plant disclosed herein may be a whole plant, or may be a plant cell, seed, or tissue, or a plant part such as leaf, stem, pollen, or cell that can be cultivated into a whole plant.

As used herein, the terms “backcross” and “backcrossing” refer to the process whereby a progeny plant is crossed back to one of its parents for one or more generations (e.g., 2 or more, 3 or more, 4 or more, 5 or more, 6 or more, or 7 or more times, etc.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on. In embodiments, at least one or more generations of progeny are identified and/or selected for the presence of the desired gene or locus (e.g., in a nucleic acid sample from the progeny plant or plant part). In embodiments, two or more generations (or even all generations) of progeny are identified and/or selected for the presence of the desired gene or locus.

As used herein, the terms “elite” and “elite line” refer to any line that has resulted from breeding and selection for desirable agronomic performance. An elite line may be substantially homozygous. Numerous elite lines are available and known to those of skill in the art, for example, those described in Table 5.

As used herein, “quantitative trait locus” (QTL) or “quantitative trait loci” (QTLs) refer to a genetic domain that effects a phenotype that can be described in quantitative terms and can be assigned a “phenotypic value” which corresponds to a quantitative value for the phenotypic trait.

As used herein the term transgenic “event” refers to a recombinant plant produced by transformation and regeneration of a plant cell or tissue with heterologous DNA, for example, an expression cassette that includes a gene of interest. The term “event” refers to the original transformant and/or progeny of the transformant that include the heterologous DNA. The term “event” also refers to progeny produced by a sexual outcross between the transformant and another corn line. Even after repeated backcrossing to a recurrent parent, the inserted DNA and the flanking DNA from the transformed parent is present in the progeny of the cross at the same chromosomal location. The term “event” also refers to DNA from the original transformant comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA. Normally, transformation of plant tissue produces multiple events, each of which represent insertion of a DNA construct into a different location in the genome of a plant cell. Based on the expression of the transgene or other desirable characteristics, a particular event is selected. Thus, in the context of this disclosure, the terms “event MIR162”, “MIR162” and “MIR162 event” are used interchangeably to refer to the event described in U.S. Pat. No. 8,232,456 and below. MIR162 plants, or cells or parts thereof, comprise the MIR162 event.

As used herein, the term “fertile” refers to a plant that is fertile enough for use in a breeding and/or seed production program. In some embodiments, fertile plants are plants that release at least about 100,000; 150,000; 200,000; 250,000, 300,000, 350,000, 400,000 or 450,000 pollen grains per tassel per day in the three-day period immediately following anther extrusion.

As used herein, the term “infertile” refers to a plant that is insufficiently fertile for use in a breeding program. In some embodiments, “infertile” plants are plants that release fewer than 25,000; 50,000; 75,000 or 100,000 pollen grains per tassel per day in the three-day period immediately following anther extrusion. “Infertile” plants may produce and/or release viable pollen grains. Indeed, in some embodiments, “infertile” plants produce and release viable pollen grains but do so a rate that is insufficient for effective use in a breeding and/or seed production program.

As used herein, the terms “increase,” “increases,” “increasing” and similar terms refer to an augmentation of at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 300% or more.

As used herein, the term “increased fertility” refers to an improvement in one or more fertility traits of a subject plant as compared to one or more control plants. A control plant, used in this context, refers to a plant having the identical genetic background as compared to the subject plant except for the lack of one or more markers/alleles associated with increased fertility. For example, an increase in fertility of an MIR162 plant having a fertile QTL is relative to a control plant and said control plant is an MIR162 plant lacking such a fertile QTL. Exemplary fertility traits include, but are not limited to, pollen count, pollen morphology, pollen production per anther, anther count, anther morphology, anthers per tassel, tassel count, tassel morphology, tassels per plant, silk count, silk morphology, silk production per plant, kernel count, kernel morphology, kernel production per ear, prevalence of kernel abortion, kernel production per plant and kernel viability. Thus, a plant that exhibits increased pollen production, increased pollen production per anther, improved pollen morphology, increased anther production, increased anther production per tassel, improved anther morphology, increased tassel production, improved tassel morphology, increased silk production, improved silk morphology, increased silk production per plant, increased kernel count, improved kernel morphology, increased kernel production per ear, decreased prevalence of kernel abortion, increased kernel production per plant, increased kernel viability, increased fertility under stress conditions (e.g., drought conditions), increased fertility under elevated daytime temperatures and/or increased fertility under elevated nighttime temperatures as compared to a control plant (e.g., a native plant/germplasm of the same species, one or both parents, a near isogenic plant that lacks one or more markers/alleles associated with increased fertility, a near isogenic plant that lacks MIR162, etc.) displays increased fertility. When used in reference to a plant part (e.g., a germplasm), the term “increased fertility” refers to an improvement in one or more fertility traits in a plant that arises from that plant part.

As used herein, the term “increased male fertility” refers to an improvement in one or more male fertility traits as compared to one or more controls (e.g., a native plant/germplasm of the same species; one or both parents, a near isogenic plant that lacks one or more markers/alleles associated with increased fertility, a near isogenic plant that lacks MIR162, a plant that contains MIR162 and one or more unfavorable alleles such as those in the Examples, etc.). Exemplary male fertility traits include, but are not limited to, pollen count, pollen morphology, pollen production per anther, anther count, anther morphology, anthers per tassel, tassel count, tassel morphology and tassels per plant. Thus, a plant that exhibits increased pollen production, increased pollen production per anther, improved pollen morphology, increased anther production, increased anther production per tassel, improved anther morphology, increased tassel production, improved tassel morphology, increased male fertility under stress conditions (e.g., drought conditions), increased male fertility under elevated daytime temperatures and/or increased male fertility under elevated nighttime temperatures as compared to a control plant (e.g., a native plant/germplasm of the same species, one or both parents, a near isogenic plant that lacks one or more markers/alleles associated with increased fertility, etc.). displays increased male fertility. When used in reference to a plant part (e.g., a germplasm), the term “increased male fertility” refers to an improvement in one or more male fertility traits in a plant that arises from that plant part.

As used herein, the term “unique” to MIR162 means distinctively characteristic of MIR162. Therefore, nucleic acids unique to event MIR162 are not found in other non-MIR162 maize plants.

The “Vip3A” class of proteins comprises, for example, Vip3Aa, Vip3Ab, Vip3Ac, Vip3Ad, Vip3Ae, VipAf, and Vip3Ag, and their homologues. “Homologue” means that the indicated protein or polypeptide bears a defined relationship to other members of the Vip3A class of proteins. “Vip3Aa20” is a Vip3A homologue unique to event MIR162. It was generated by spontaneous mutations introduced into the maize-optimized Vip3Aa19 gene comprised in pNOV1300 (SEQ ID NO: 234) during the plant transformation process as described in U.S. Pat. No. 8,232,456 and below.

As used herein, the phrase “associated with” as in, for example, “a marker associated with a trait” refers to when the marker and trait are linked such that the presence of the marker is an indicator of the presence and/or extent the desired trait or trait form will occur in a plant/plant part comprising the marker. Similarly, a marker is “associated with” an allele when the marker and allele are linked such that the presence of the marker is an indicator of the presence of the allele in a plant/plant part comprising the marker. For example, “a marker associated with increased male fertility” refers to a marker whose presence or absence can be used to predict whether and/or to what extent a plant/plant part will display increased male fertility.

As used herein, “SNP” or “single nucleotide polymorphism” means a sequence variation that occurs when a single nucleotide (A, T, C, or G) in the genome sequence is altered or variable. “SNP markers” exist when SNPs are mapped to sites on the genome.

As used herein, the terms “allele of interest,” and “favorable allele” are used interchangeably to refer to an allele that is linked to a desired trait, e.g., increased fertility. An “allele of interest” may be associated with either an increase or decrease of or in a given trait, depending on the nature of the desired phenotype, and may be associated with a change in morphology, color, etc. In some embodiments of the present invention, the “allele of interest” is associated with increased male fertility and may therefore be used as a marker to identify, select and/or produce fertile maize plants; to predict whether and/or to what extent a maize plant will be fertile; to reduce the costs associated with breeding and/or seed production programs; and/or to increase the efficiency of breeding and/or seed production programs.

As used herein, the term “unfavorable allele” refers to an allele that segregates with an unfavorable plant phenotype, therefore providing the benefit of identifying plants that can be removed from a breeding program or planting.

As used herein, “fertile QTL” refers to a QTL comprising a marker locus or marker loci with one or more alleles that, in combination, are associated with baseline or increased fertility of MIR162 plants, e.g., as measured by seed set and/or yield, relative to control plants that do not contain MIR162 or increased fertility relative to plants that contain MIR162 but also contain infertile QTLs. These alleles are referred to as “favorable allele” in this application.

As used herein, “infertile QTL” refers to a QTL comprising a marker locus or marker loci with one or more alleles that, in combination, are associated with decreased fertility of MIR162 plants, e.g., as measured by seed set and/or yield, relative to control plants that do not contain MIR162. These alleles are referred to as “unfavorable allele” in this application.

As used herein, the term “plant” may refer to any suitable plant, including, but not limited to, spermatophytes (e.g., angiosperms and gymnosperms) and embryophytes (e.g., bryophytes, ferns and fern allies). In some embodiments, the plant is a monocotyledonous (monocot) plant such as a rice, maize, wheat, barley, sorghum, millet, oat, triticale, rye, buckwheat, fonio, quinoa, sugar cane, bamboo, banana, ginger, onion, lily, daffodil, iris, amaryllis, orchid, canna, bluebell, tulip, garlic, secale, einkorn, spelt, emmer, durum, kamut, grass (e.g., gramma grass), teff, milo, flax, Tripsacum sp., or teosinte plant. In some embodiments, the plant is a dicotyledonous (dicot) plant such as a blackberry, raspberry, strawberry, barberry, bearberry, blueberry, coffee berry, cranberry, crowberry, currant, elderberry, gooseberry, goji berry, honeyberry, lemon, lime, lingonberry, mangosteen, orange, pepper, persimmon, pomegranate, prune, cotton, clover, acai, plum, peach, nectarine, cherry, guava, almond, pecan, walnut, amaranth, apple, sweet pea, pear, potato, soybean, sugar beet, sunflower, sweet potato, tamarind, tea, tobacco or tomato plant.

As used herein, the term “plant cell” refers to a cell existing in, taken from and/or derived from a plant (e.g., derived from a plant cell/tissue culture). Thus, the term “plant cell” may refer to an isolated plant cell, a plant cell in a culture, a plant cell in an isolated tissue/organ and/or a plant cell in a whole plant.

As used herein, the term “plant part” refers to at least a fragment of a whole plant or to a cell culture or tissue culture derived from a plant. Thus, the term “plant part” may refer to plant cells, plant tissues and plant organs, as well as cell/tissue cultures derived from plant cells, plant tissues and plant cultures. Embodiments of the present invention may comprise and/or make use of any suitable plant part, including, but not limited to, anthers, branches, buds, calli, clumps, cobs, cotyledons, ears, embryos, filaments, flowers, fruits, husks, kernels, leaves, lodicules, ovaries, palea, panicles, pedicels, pods, pollen, protoplasts, roots, root tips, seeds, silks, stalks, stems, stigma, styles, and tassels. In some embodiments, the plant part is a plant germplasm.

As used herein, the term “polymorphism” refers to a variation in the nucleotide sequence at a locus, where said variation is too common to be due merely to a spontaneous mutation. A polymorphism generally has a frequency of at least about 1% in a population. A polymorphism can be a single nucleotide polymorphism (SNP), or an insertion/deletion polymorphism, also referred to herein as an “indel.” Additionally, the variation can be in a transcriptional profile or a methylation pattern. The polymorphic site or sites of a nucleotide sequence can be determined by comparing the nucleotide sequences at one or more loci in two or more germplasm entries.

As used herein, the terms “polypeptide,” “peptide” and “protein” refer to a polymer of amino acid residues. The terms encompass amino acid polymers in which one or more amino acid residue is an artificial chemical analogue of a corresponding naturally occurring amino acid, as well as to naturally occurring amino acid polymers.

The term “progeny,” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

### II. MIR162 Plants and Fertility

MIR162 plants are described in U.S. Pat. No. 8,232,456. Methods of generating and identifying MIR162 plants are described in Example 1 of U.S. Pat. No. 8,232,456. A MIR162 plant can be hemizygous or homozygous for the MIR162 event. The MIR162 plants comprise unique nucleic acid sequences that are not found in plants not comprising the MIR162 event. The unique nucleic acid sequences include junction sequences, a Vip3Aa20 coding sequence, which encodes a Vip3Aa20 insecticidal protein that confers insect resistance, and a pmi coding sequence, which encodes a PMI protein that confers upon corn cells the ability to utilize mannose as a carbon source. A MIR162 plant typically comprises nucleic acid sequences one or more of SEQ ID NO: 224-231. In some embodiments, the MIR162 plant comprises nucleic acid sequences SEQ ID NO: 224 (Vip3A coding sequence), SEQ ID NO: 227 (the 5′ genome-insert junction sequence), and 228 (the 3′ genome-insert junction sequence). As disclosed in U.S. Pat. No. 8,232,456, seeds from MIR162 plants were deposited in accordance with the Budapest Treaty at the American Type Culture Collection (ATCC), 1801 University Boulevard, Manassas, Virginia, 20110 under ATCC Accession No. PTA-6188.

Introducing the MIR162 event into maize plants is effective in controlling lepidopteran insect pests including, but not limited to, black cutworm (BCW, Agrotis ipsilon), fall armyworm (FAW, Spodoptera frugiperda), tobacco budworm (TBW, Heliothis virescens), sugarcane borer (SCB, Diatraea saccharalis), lesser cornstalk borer (LCB, Elasmopalpus lignosellus), corn earworm (CEW, Helicoverpa zea), and western bean cutworm (WBCW, Striacosta albicosta).

However it has been observed that the MIR162 event is associated with unsuccessful event conversion in some maize backgrounds, i.e., introduction of the MIR162 event into certain maize backgrounds results in the plants having reduced, in some cases severely reduced, male fertility. For example, over a three year period, the overall unsuccessful event conversion rate for MIR162 across maize backgrounds was observed to be about 41-47% (Table 2).

### 1. Fertile QTLs

In one aspect, the disclosure provides various QTLs with marker loci that are associated with fertility of MIR162 plants. These QTLs comprise marker loci associated with fertility of MIR162 plants. These QTLs include, but are not limited to, a QTL on chromosome 5 that comprises one or more marker loci of SEQ ID NOs: 1-5; a QTL on chromosome 2 that comprises one or more of SEQ ID NO: 6-55; a QTL on chromosome 6 that comprises one or more of SEQ ID NO: 56-89; a QTL on chromosome 7 that comprises one or more of SEQ ID NO: 90-131; a a QTL located on chromosome 8 comprising SEQ ID NO: 255; and a QTL on chromosome 9 that comprises one or more of SEQ ID NO: 132-223. One or more of these QTLs can be used in the methods and comprised in the plants described in this disclosure. One or more of any of these QTLs can be used in the methods and comprised in the plants described in this disclosure.

Also provided by the disclosure are one or more fertile QTLs, i.e., QTLs comprising marker loci with alleles that are associated with increased fertility of MIR162 plants having these QTLs. In some embodiments, the fertile QTL is located on chromosome 5 and comprises one or more marker loci of SEQ ID NOs: 1-5, with the position number 100 of the sequences being the favorable alleles shown in Table 3, i.e., G, C, G, A, and C, respectively. In some embodiments, the fertile QTL comprises SEQ ID NO: 2, with the position number 100 of the sequence being C. In some embodiments, the fertile QTL comprises SEQ ID NO: 2, with the position number 100 of the sequence being C and one or more of SEQ ID NO: 1 and 3-5, with the position number 100 of the sequences being G, G, A, and C, respectively. In some embodiments, the fertile QTL is on chromosome 2, comprising one or more of SEQ ID NO: 6-55, the position number 100 of the sequences being the favorable alleles shown in Table 4. In some embodiments, the fertile QTL is on chromosome 6, comprising one or more of SEQ ID NO: 56-89, the position number 100 of the sequences being the favorable alleles shown in Table 4. In some embodiments, the fertile QTL is on chromosome 7, comprising one or more of SEQ ID NO: 90-131, the position number 100 of the sequences being the favorable alleles shown in Table 4. In some embodiments, the fertile QTL is on chromosome 8 and comprises SEQ ID NO: 255, with position 100 of the sequence being the favorable allele shown in Table 9. In some embodiments, the fertile QTL is on chromosome 9 and comprises one or more of SEQ ID NO: 132-223, with position 100 of the sequences being the favorable alleles shown in Table 4.

In some embodiments, the one or more fertile QTLs comprise a combination of SEQ ID NO: 2, 52, 88, 139, and 94, wherein position number 100 of each sequence, respectively and in combination, comprises one of the following: (i) C, A, A, T, and T; (ii) C, A, A, C, and T; (iii) C, A, G, C, and T; (iv) C, G, A, C, and T; (v) C, G, G, C, and T; (vi) C, G, G, C, and C; (vii) A, A, A, T, and T; (viii) A, A, G, T, and T; (ix) A, A, A, C, and T; (x) A, G, A, T, and T; (xi) A, G, A, C, and T, or (xii) A, A, G, C, and T.

In some embodiments, the one or more fertile QTLs comprise one of the following: (i) at least one of SEQ ID NO: 2 with a C at position 100, SEQ ID NO: 52 with an A at position 100, SEQ ID NO: 88 with an A at position 100, SEQ ID NO: 139 with a T at position 100, or SEQ ID NO: 94 with a T at position 100; (ii) at least one of SEQ ID NO: 2 with a C at position 100, SEQ ID NO: 52 with an A at position 100, SEQ ID NO: 88 with an A at position 100, SEQ ID NO: 139 with a C at position 100, or SEQ ID NO: 94 with a T at position 100; (iii) at least one of SEQ ID NO: 2 with a C at position 100, SEQ ID NO: 52 with an A at position 100, SEQ ID NO: 88 with a G at position 100, SEQ ID NO: 139 with a C at position 100, or SEQ ID NO: 94 with a T at position 100; (iv) at least one of SEQ ID NO: 2 with a C at position 100, SEQ ID NO: 52 with a G at position 100, SEQ ID NO: 88 with an A at position 100, SEQ ID NO: 139 with a C at position 100, or SEQ ID NO: 94 with a T at position 100; (v) at least one of SEQ ID NO: 2 with a C at position 100, SEQ ID NO: 52 with a G at position 100, SEQ ID NO: 88 with a G at position 100, SEQ ID NO: 139 with a C at position 100, or SEQ ID NO: 94 with a T at position 100; (vi) at least one of SEQ ID NO: 2 with a C at position 100, SEQ ID NO: 52 with a G at position 100, SEQ ID NO: 88 with a G at position 100, SEQ ID NO: 139 with a C at position 100, or SEQ ID NO: 94 with a C at position 100; (vii) at least one of SEQ ID NO: 2 with an A at position 100, SEQ ID NO: 52 with an A at position 100, SEQ ID NO: 88 with an A at position 100, SEQ ID NO: 139 with a T at position 100, or SEQ ID NO: 94 with a T at position 100; (viii) at least one of SEQ ID NO: 2 with an A at position 100, SEQ ID NO: 52 with an A at position 100, SEQ ID NO: 88 with a G at position 100, SEQ ID NO: 139 with a T at position 100, or SEQ ID NO: 94 with a T at position 100; (ix) at least one of SEQ ID NO: 2 with an A at position 100, SEQ ID NO: 52 with an A at position 100, SEQ ID NO: 88 with an A at position 100, SEQ ID NO: 139 with a C at position 100, or SEQ ID NO: 94 with a T at position 100; (x) at least one of SEQ ID NO: 2 with an A at position 100, SEQ ID NO: 52 with a G at position 100, SEQ ID NO: 88 with an A at position 100, SEQ ID NO: 139 with a T at position 100, or SEQ ID NO: 94 with a T at position 100; (xi) at least one of SEQ ID NO: 2 with an A at position 100, SEQ ID NO: 52 with a G at position 100, SEQ ID NO: 88 with an A at position 100, SEQ ID NO: 139 with a C at position 100, or SEQ ID NO: 94 with a T at position 100; or (xii) at least one of SEQ ID NO: 2 with an A at position 100, SEQ ID NO: 52 with an A at position 100, SEQ ID NO: 88 with a G at position 100, SEQ ID NO: 139 with a C at position 100, or SEQ ID NO: 94 with a T at position 100.

In some embodiments, the one or more fertile QTLs comprise SEQ ID NO: 2, wherein position number 100 is C or A; SEQ ID NO: 52, wherein position number 100 is A or G; SEQ ID NO: 88, wherein position number 100 is A or G; SEQ ID NO: 139, wherein position number 100 is T or C; and SEQ ID NO: 94, wherein position number 100 is T or C.

In some embodiments, the one or more fertile QTLs comprise one or more of SEQ ID NO: 2, wherein position number 100 is C or A; SEQ ID NO: 52, wherein position number 100 is A or G; SEQ ID NO: 88, wherein position number 100 is A or G; SEQ ID NO: 139, wherein position number 100 is T or C; or SEQ ID NO: 94, wherein position number 100 is T or C.

Plants having infertile QTLs are expected to have reduced male fertility in MIR162 plants, therefore detecting infertile QTLs in plants can be a criterion for excluding plants from being used to generate MIR162 plants. In some embodiments, the infertile QTLs include SEQ ID NO: 1-5, with position number 100 of the sequences being the unfavorable alleles in Table 3, i.e., A, A, A, G, and A, respectively. In some embodiments, the infertile QTL is on chromosome 2, comprising one or more of SEQ ID NO: 6-55, the position number 100 of the sequences being the unfavorable alleles shown in Table 4. In some embodiments, the infertile QTL is on chromosome 6, comprising one or more of SEQ ID NO: 56-89, the position number 100 of the sequences being the unfavorable alleles shown in Table 4. In some embodiments, the infertile QTL is on chromosome 7, comprising one or more of SEQ ID NO: 90-131, the position number 100 of the sequences being the unfavorable alleles shown in Table 4. In some embodiments, the infertile QTL is on chromosome 8, comprising SEQ ID NO: 255, with position 100 of the sequence being the unfavorable alle shown in Table 9. In some embodiments, the infertile QTL is on chromosome 9 and comprises one or more of SEQ ID NO: 132-223, with position 100 of the sequences being the unfavorable alleles shown in Table 4.

In some embodiments, the one or more infertile QTLs comprise a combination of SEQ ID NO: 2, 52, 88, 139, and 94, wherein position number 100 of each sequence, respectively and in combination, is the one of the following: (i) A, G, G, T, and T; (ii) A, G, G, C, and T; (iii) A, A, G, C, C; (iv) A, G, G, T, C; or (v) A, G, G, C, C.

In some embodiments, the one or more infertile QTLs comprise one of the following: (i) at least one of SEQ ID NO: 2 with an A at position 100, SEQ ID NO: 52 with a G at position 100, SEQ ID NO: 88 with a G at position 100, SEQ ID NO: 139 with a T at position 100, or SEQ ID NO: 94 with a T at position 100; (ii) at least one of SEQ ID NO: 2 with an A at position 100, SEQ ID NO: 52 with a G at position 100, SEQ ID NO: 88 with a G at position 100, SEQ ID NO: 139 with a C at position 100, or SEQ ID NO: 94 with a T at position 100; (iii) at least one of SEQ ID NO: 2 with an A at position 100, SEQ ID NO: 52 with an A at position 100, SEQ ID NO: 88 with a G at position 100, SEQ ID NO: 139 with a C at position 100, or SEQ ID NO: 94 with a C at position 100; (iv) at least one of SEQ ID NO: 2 with an A at position 100, SEQ ID NO: 52 with a G at position 100, SEQ ID NO: 88 with a G at position 100, SEQ ID NO: 139 with a T at position 100, or SEQ ID NO: 94 with a C at position 100; (v) at least one of SEQ ID NO: 2 with an A at position 100, SEQ ID NO: 52 with a G at position 100, SEQ ID NO: 88 with a G at position 100, SEQ ID NO: 139 with a C at position 100, or SEQ ID NO: 94 with a C at position 100.

In some embodiments, the presence of the QTL is confirmed by a locus that is closely linked to the QTLs, for example a locus that is within about 20 centimorgan (cM), 15 cM, 10 cM, 5 cM, 4 cM, 3 cM, 2 cM, 1 cM, 0.5 cM, or less than 0.5 cM of the QTL.

2. TE Associated with Increased Fertility

In another aspect, disclosed herein is a transposable element (TE) that is associated with fertility of MIR162 plants. In some embodiments, the TE is located on chromosomal 5. In some embodiments, the TE is located on Chromosome 5: 130436801-130439201 in B73_ver5. In one embodiment, the TE comprises SEQ ID NO: 232 or 233. In some embodiments, the TE comprises a sequence sharing at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 232 or 233. In one illustrative embodiment as shown in Table 8, the presence of the TE (SEQ ID NO: 232 or 233) on chromosome 5 was shown to positively correlate with conversion success.

In some embodiments, plants with one or more fertile QTLs and/or a TE disclosed herein are selected and used to cross with MIR162 plants to produce further MIR162 plants, e.g., hybrid plants. In some embodiments, the MIR162 event can be introduced to the plants with one or more fertile QTLs disclosed herein to produce MIR162 plants having increased fertility. In some embodiments, the MIR162 event can be introduced to the plants with a TE disclosed herein into Chromosome 2 or 5 at the location disclosed above to produce MIR162 plants having increased fertility. In some embodiments, the genome of MIR162 plants are edited to remove the unfavorable alleles and introduce favorable allele and/or the TE to increase fertility. These approaches are further described in detail below.

### III. Creating MIR162 Plants with Fertile QTLs

### 1. Crossing

In one approach, MIR162 plants with one or more fertile QTLs and/or a TE associated with increased fertility can be created by crossing a first maize plant comprising the fertile QTL(s) and/or the TE with a second maize plant to produce a plurality of progeny plants. In some embodiments, the first maize plant is an MIR162 plant. In some embodiments the second maize plant is an MIR162 plant. In some embodiments, both the first and second maize plant are MIR162 plants. In some embodiments, the MIR162 maize plant is obtained from growing a seed deposited at the ATCC under the accession No. PTA-6188. The crosses of the first and second maize plants produce a plurality of progeny plants.

In some embodiments, progeny plants are genotyped to confirm (i) the presence of fertile QTLs and/or the absence of infertile QTLs and/or (ii) the presence or absence of the TE associated with increased fertility using the methods disclosed in the section above.

In some embodiments, retention of the MIR162 event in these progeny plants can be confirmed by detecting the presence of one or more nucleic acids (e.g., one or more nucleic acids encoding Vip3Aa) or proteins that are unique to the MIR162 event and/or by detecting the insect resistance in the plant due to the presence of the MIR162 event. In one illustrative example, the method of detecting one or more nucleic acids that are unique to the MIR162 event comprises (a) contacting the sample with a pair of polynucleotide primers that, when used in a nucleic acid amplification reaction with genomic DNA from the MIR162 event produces an amplicon that is diagnostic for the MIR162 event; (b) performing a nucleic acid amplification reaction, thereby producing the amplicon; and (c) detecting the amplicon. In one aspect of this embodiment, the amplicon comprises a nucleotide sequence selected from the group consisting of SEQ ID NO: 224-231 and the complements thereof. Other example methods include use of probes to detect one or more nucleic acids that are unique to the MIR162 event or sequencing all or part of the genome of the plant to detect one or more nucleic acids that are unique to the MIR162 event. In another illustrative example, retention of the MIR162 event in these progeny plants can be confirmed by detection of Vip3A protein (e.g, Vip3Aa), e.g., by mass spectrometry or by enzyme-linked immunosorbent assay (ELISA). In some instances, Vip3A protein (e.g., Vip3Aa) is detected.

These progeny plants comprising the fertile QTL(s) and/or a TE associated with increased fertility and MIR162 events can be backcrossed with a parent plant comprising the MIR162 events and/or the fertile QTL(s) and/or the TE. The F1 progeny plants from the cross can be assayed for one or more fertile QTLs or a TE associated with increased fertility disclosed herein. A selected F1 progeny plant is then backcrossed with the parent MIR162 plant (recurrent parent). Plants from the BC1 generation are also genotyped for the one or more fertile QTLs or a TE associated with increased fertility disclosed herein. After multiple rounds of backcrossing (e.g., 5-7 generations) with the recurrent parent line, a new elite maize inbred line is obtained comprising both one or more fertile QTLs or a TE associated with increased fertility disclosed herein and the MIR162 event). In some embodiments, such inbred plants can be used as parent plants, e.g., as male parent plants, to create hybrid plants, which are a preferred commercial embodiment of maize in most countries due to heterosis of the hybrids.

### 2. Transformation

In another approach, MIR162 plants with fertile QTL(s) can be created by selecting a maize plant comprising one or more fertile QTLs or a TE associated with increased fertility as disclosed herein, introducing a MIR162 event into said maize plant, and growing the plant to result in a MIR162 plant comprising these fertile QTL(s) and/or TE. In some embodiments, the maize plant comprising one or more fertile QTL(s) and/or TE disclosed herein is a known maize line, for example, any one of the maize lines listed in Table 5.

The MIR162 event can be introduced into corn plants by transforming nucleic acid sequences that are unique to the MIR162 event into corn plants using methods well known in the art. See Section II above, entitled “Vip3A plants and fertility.” One illustrative example is described in Example 1 of U.S. Pat. No. 8,232,456.

### 3. Genome Editing

In another approach, one or more fertile QTLs and a TE associated with increased fertility can be introduced into a maize plant by genome editing. Various embodiments of the methods described herein use genome editing. In some embodiments, genome editing is used to modify the genome of a plant to produce plants having one or more of the fertile QTLs and/or a TE associated with increased fertility to increase the fertility of MIR162 plants. In some embodiments, the genome of an MIR162 plant is edited to replace the one or more unfavorable alleles in SEQ ID NOs: 1-223 or 255 with the corresponding favorable alleles in Tables 3, 4 or 9. For example, genome editing can be performed in a maize plant to replace the unfavorable allele A at position 100 in SEQ ID NO: 2 with the favorable allele C. In some embodiments, genome editing is used to introduce the favorable allele from any of the loci in Table 4, such as at chromosome 2 at position 243529534, chromosome 5 at position 129259011, chromosome 6 at position 158572433, chromosome 7 at position 134444680, or chromosome 9 at position 8013654, or an allelic combination in Table 4, into a maize plant that does not contain such allele(s) or allelic combination and would otherwise not succeed in MIR162 event conversion. It is expected that introduction of the favorable allele(s) or allelic combination will improve the likelihood of a successful MIR162 event conversion.

In some embodiments, the genome of a MIR162 plant is edited to insert a TE associated with increased fertility into the genome. In some embodiments, the TE is inserted on chromosome 5. In some embodiments, the TE is inserted into Chromosome 5 at positions 130436801-130439201 in B73 ver5. In one embodiment, the TE comprises SEQ ID NO: 232 or 233.

Methods of editing genomes are well known in the art. Such methods include, but are not limited to, meganucleases designed against the plant genomic sequence of interest CRISPR-Cas9, TALENs, and other technologies for precise editing of genomes (Feng, et al. Cell Research 23: 1229-1232, 2013, WO 2013/026740); Cre-lox site-specific recombination; FLP-FRT recombination (Li et al. (2009) Plant Physiol 151:1087-1095); Bxbl-mediated integration (Yau et al. Plant J (2011) 701: 147-166); zinc-finger mediated integration (Wright et al. (2005) Plant J 44:693-705); Cai et al. (2009) Plant Mol Biol 69:699-709); homologous recombination (Lieberman-Lazarovich and Levy (2011) Methods Mol Biol 701:51-65); prime editing and transposases (Anzalone, A. et al. (2020) Nat Biotechnol. 38(7):824-844); translocation; and inversion.

In some embodiments, provided herein are plants transformed with and expressing genome-editing machinery as described above, which, when crossed with a target plant, result in genome editing in the target plant.

In general, genome editing may involve transient, inducible, or constitutive expression of the genome editing components or systems in the target plant or bombardment of the genome editing components directly into the plant. Genome editing may involve genomic integration or episomal presence of the genome editing components or systems.

Genome editing generally refers to the use of a site-directed nuclease (including but not limited to CRISPR/Cas, zinc fingers, meganucleases, and the like) to cut a nucleotide sequence at a desired location. This may be to cause a deletion or an insertion/deletion (“indel”) mutation, (i.e., “SDN1”), a base edit (i.e., “SDN2”), or allele insertion or replacement (i.e., “SDN3”). SDN2 or SDN3 genome editing may comprise the provision of one or more recombination templates (e.g., in a vector) comprising a gene sequence of interest that can be used for homology directed repair (HDR) within the plant (i.e., to be introduced into the plant genome). In some embodiments, the gene or allele of interest is one that is able to confer increased fertility as disclosed herein. The recombination template can be introduced into the plant to be edited either through transformation or through breeding with a donor plant comprising the recombination template. Breaks in the plant genome may be introduced within, upstream, and/or downstream of a target sequence. In some embodiments, a double strand DNA break is made within or near the target sequence locus.

In some embodiments, breaks are made upstream and downstream of the target sequence locus, which may lead to its excision from the genome. In some embodiments, one or more single strand DNA breaks (nicks) are made within, upstream, and/or downstream of the target sequence (e.g., using a nickase Cas9 variant). Any of these DNA breaks, as well as those introduced via other methods known to one of skill in the art, may induce HDR. Through HDR, the target sequence is replaced by the sequence of the provided recombination template comprising a polynucleotide of interest, e.g., a fertile QTL and/or TE. By designing the system such that one or more single strand or double strand breaks are introduced within, upstream, and/or downstream of the corresponding region in the genome of a plant not comprising the gene sequence of interest, this region can be replaced with the template.

In some embodiments, mutations in the genes of interest described herein may be generated without the use of a recombination template via targeted introduction of DNA double strand breaks. Such breaks may be repaired through the process of non-homologous end joining (NHEJ), which can result in the generation of small insertions or deletions (indels) at the repair site. Such indels may lead to frameshift mutations causing premature stop codons or other types of loss-of-function mutations in the targeted genes.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261; 6,607,882; 6,746,838; 6,794,136; 6,824,978; 6,866,997; 6,933,113; and 6,979,539.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein and as described in foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cas12a).

The Cas protein as referred to herein, such as but not limited to Cas9, Cas12a (formerly referred to as Cpf1), Cas12b (formerly referred to as C2c1), Cas13a (formerly referred to as C2c2), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is Cas12a, optionally from Acidaminococcus sp., such as Acidaminococcus sp. BV3L6 Cpf1 (AsCas12a) or Lachnospiraceae bacterium Cas12a, such as Lachnospiraceae bacterium MA2020 or Lachnospiraceae bacterium MD2006 (LBCas12a). See U.S. Pat. No. 10,669,540, incorporated herein by reference in its entirety. Alternatively, the Cas12a protein may be from Moraxella bovoculi AAX08_00205 [Mb2Cas12a] or Moraxella bovoculi AAX11_00205 [Mb3Cas12a]. See WO 2017/189308, incorporated herein by reference in its entirety. In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b. Other Cas enzymes are available to a person skilled in the art.

Genome editing methods and compositions are also disclosed in U.S. Pat. Nos. 10,519,456 and 10,285,348 82, the entire content of which is herein incorporated by reference.

The gene-editing machinery (e.g., the DNA modifying enzyme) introduced into the plants can be controlled by any promoter that can drive recombinant gene expression in plants. In some embodiments, the promoter is a constitutive promoter. In some embodiments, the promoter is a tissue-specific promoter, e.g., a pollen-specific promoter or a sperm cell specific promoter, a zygote specific promoter, or a promoter that is highly expressed in sperm, eggs and zygotes (e.g., prOsActin1). Suitable promoters are disclosed in U.S. Pat. No. 10,519,456, the entire content of which is herein incorporated by reference.

In some embodiments, the guide RNA and the Cas protein (or any other suitable nucleases) may be delivered in DNA form, e.g., in a suitable vector that can be introduced into a yeast cell. Generally, DNA encoding the gRNA is cloned into a vector downstream of a promoter for expression. The sgRNA and Cas may be expressed from the same vector of the system or from different vectors. In some embodiments, the vectors are separately transformed into the maize plant to induce genome editing. In some embodiments, the coding sequence for Cas9 and the coding sequence for the sgRNA are ligated into a single vector, which is then transformed into the maize plant to induce genomic modification. The Cas9 vector and the sgRNA vector typically contains a selection marker, for example, spectinomycin, for identification of transformants comprising the genome editing machinery.

In some embodiments, the method of introducing desired genomic modifications comprises using a first maize plant expressing a DNA modification enzyme and at least one optional guide nucleic acid as described above to pollinate a target plant comprising genomic DNA to be edited.

### IV. Detecting Presence of QTLs and Tes in Plants

Also provided herein are methods of detecting the presence of a fertile QTL and/or a TE associated with increased fertility (“target nucleic acid sequence”) that can increase the fertility of MIR162 plants. A target sequence, e.g., a particular allele (e.g., a favorable allele in Table 3) can be detected by methods including, but not limited to, nucleic acid sequencing, hybridization methods, amplification methods (e.g., PCR-based sequence specific amplification methods), detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), detection of amplified fragment length polymorphisms (AFLPs), detection of expressed sequence tags (ESTs). SSR markers derived from EST sequences and/or randomly amplified polymorphic DNA (RAPD).

In one aspect the detection of the target nucleic acid sequence can be facilitated through the use of nuclear acid amplification methods. Such methods specifically increase concentration of poly nucleotides that span the polymorphic site, or include that site and sequences located either distal or proximal to it. Such amplified molecules can be readily detected by gel electrophoresis fluorescent detection methods or other means.

In another aspect, a target nucleic acid sequence can be detected by probe ligation methods as provided in U.S. Pat. No. 5,800,944 where sequence of interest is amplified and hybridized to probes followed by ligation to detect a labeled part of the probe.

Target nucleic acid sequence can also be detected by probe linking methods, employing at least one pair of probes having sequences homologous to adjacent portions of the target nucleic acid sequence and having side chains which non-covalently bind to form a stem upon base pairing of the probes to the target nucleic acid sequence. See, U.S. Pat. No. 5,616,464.

Polymorphism in nucleic acid sequences (e.g., SNPs disclosed in Table 3 or Table 4 or Table 9) can be detected using various methods. In one embodiment, polymorphism in nucleic acid sequences is detected by hybridization to allele specific oligonucleotide probes.

In one exemplary method, single or multiple nucleotide variations in nucleic acid sequence are detected in nucleic acids by a process in which the sequence containing the nucleotide variation is amplified, spotted on a membrane and treated with a labeled sequence-specific oligonucleotide probe. See, U.S. Pat. Nos. 5,468,613 and 5,217,863 In another embodiment, polymorphism in nucleic acid sequences is detected using microarray, in which oligonucleotide probe sets are assembled in an overlapping fashion to represent a single sequence such that a difference in the target sequence at one point would result in partial probe hybridization (Borevitz et al., Large-scale identification of single-feature polymorphism in complex genomes. Genome Research, 13:513-523 (2003).

Other exemplary methods for detecting polymorphism in nucleic acid sequences include single base extension (SBE) methods. Examples of SBE methods include, but are not limited, to those provided in U.S. Pat. Nos. 6,004,744; 6,013,431; 5,595,890; 5,762,876; and 5,945,283. SBE methods are based on extension of a nucleotide primer that is adjacent to a polymorphism to incorporate a detectable nucleotide residue upon extension of the primer. If the polymorphism is present on the template, one of the labeled dideoxynucleosidetriphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

In another exemplary method for detecting polymorphisms, polymorphism in nucleic acid sequences can be detected by methods provided in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g., by Forster-type energy transfer. During PCR, forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another aspect, a target nucleic acid sequence can be detected using nucleic acid sequencing technologies. Methods for nucleic acid sequencing are known in the art and include technologies provided by 454 Life Sciences (Branford, Conn.), Agencourt Bioscience (Beverly, Mass.), Applied Biosystems (Foster City, Calif.), LI-COR Biosciences (Lincoln, Nebr.), NimbleGen Systems (Madison, Wis.), Illumina (San Diego, Calif), Pac-Bio (Menlo Park, Calif) and VisiGen Biotechnologies (Houston, Tex.). Such nucleic acid sequencing technologies comprise formats such as parallel bead arrays, sequencing by ligation, capillary electrophoresis, electronic microchips, “biochips,” microarrays, parallel microchips, and single-molecule arrays.

Accordingly, provided herein are kits for genotyping a plant to detect the presence of favorable alleles in the QTLs disclosed herein, or to detect the presence of a TE that is associated with increased fertility. The kit may comprise one of more reagents disclosed above, for example, the kit may comprise reagents used for detecting the presence of one or more marker loci of SEQ TD Nos: 1-223 or 255; and/or a transposon element (TE) of SEQ ID NO: 232 or 233. In some embodiments, the kit comprises one or more of the following: primers for sequencing the one or more marker loci and/or the TE (for example, primers comprising at least 10 consecutive nucleotides that are complementary to any one of SEQ TD Nos: 1-223 or 255 or 232); primers for amplifying the one or more marker loci and/or the TE (for example, primers comprising at least 10 consecutive nucleotides that are complementary to any one of SEQ ID Nos: 1-223 or 255 or 232), or a nucleic acid molecule that is complementary to one or more marker loci. amplify and/or sequence one or more marker loci and/or the TE. can be used for amplifying and/or sequencing the alleles. Provided below in Table 1 are example primers and probes which can be used, e.g., in a TAQMAN assay, to detect one or more marker loci.

TABLE 1
Primer and Probes for certain marker loci

| | Assay component | | | Detected |
|---|---|---|---|---|
| SNP Code | name | DNA sequence | Probe label | nucleotide |
| 9_12621982 | SM12131 Fam probe | ATGCTTTTCGAGCATGC | 6-carboxyfluorescein | A |
| | | (SEQ ID NO: 235) | | |
| 9_12621982 | SM12131 Tet probe | CATATGCTTTTCGGGCA | tetrachlorofluorescein | G |
| | | TG (SEQ ID NO: 236) | | |
| 9_12621982 | SM12131 Forward | TGTCCGCTTCTTGGCTC | | |
| | Primer | TA (SEQ ID NO: 237) | | |
| 9_12621982 | SM12131 Reverse | ACTGGGCCGGTCATCA | | |
| | Primer | TTCTTCT (SEQ ID | | |
| | | NO: 238) | | |
| 9_12627976 | SM4554A1 Fam probe | ACTAGGCCAACCAAGC | 6-carboxyfluorescein | A |
| | | C (SEQ ID NO: 239) | | |
| 9_12627976 | SM4554A2 Tet probe | ACTAGGCCACCCAAGC | tetrachlorofluorescein | C |
| | | (SEQ ID NO: 240) | | |
| 9_12627976 | SM4554 Forward | TCACATGGGCCACGTTT | | |
| | Primer | TC (SEQ ID NO: 241) | | |
| 9_12627976 | SM4554 Reverse | GTTGGGCTAGCGTGGG | | |
| | Primer | AAT (SEQ ID NO: 242) | | |
| 9_12633481 | SM12130 Fam probe | TGCCTAATAATTTCTTA | 6-carboxyfluorescein | A |
| | | TATTTCT (SEQ ID | | |
| | | NO: 243) | | |
| 9_12633481 | SM12130 Tet probe | ATGTGCCTAATAATTTC | tetrachlorofluorescein | G |
| | | TTGTATTT (SEQ ID | | |
| | | NO: 244) | | |
| 9_12633481 | SM12130 Forward | AATTTTCTGTAAAAGTT | | |
| | primer | GAGTATCA (SEQ ID | | |
| | | NO: 245) | | |
| 9_12633481 | SM12130 Reverse | GCGCAGAATGGGAATG | | |
| | primer | TC (SEQ ID NO: 246) | | |
| 9_12648983 | SM3788A1 Fam probe | TAGAGCTTACCGACTGT | 6-carboxyfluorescein | G |
| | | A (SEQ ID NO: 247) | | |
| 9_12648983 | SM3788A2 Tet probe | TAGAGCTTACTGACTGT | tetrachlorofluorescein | A |
| | | A (SEQ ID NO: 248) | | |
| 9_12648983 | SM3788 Forward | CGTAGGCTTCCCGCACT | | |
| | primer | ATG (SEQ ID NO: 249) | | |
| 9_12648983 | SM3788 Reverse | AGGCTTTGTTGTGGCCT | | |
| | primer | GGT (SEQ ID NO: 250) | | |
| 9_12676927 | SM4989A1 Fam probe | TCCACACCAACCTGA | 6-carboxyfluorescein | A |
| | | (SEQ ID NO: 251) | | |
| 9_12676927 | SM4989A2 Tet probe | CACACCAGCCTGAAC | tetrachlorofluorescein | G |
| | | (SEQ ID NO: 252) | | |
| 9_12676927 | SM4989 Forward | GCACCGCATGGTCTCC | | |
| | primer | AC (SEQ ID NO: 253) | | |
| 9_12676927 | SM4989 Reverse | GAGGGCTTCCACAACC | | |
| | primer | TGAT (SEQ ID NO: 254) | | |

### V. Other Polynucleotides and Polypeptides of Interest

In some embodiments, the MIR162 plants with one or more fertile QTLs produced herein can be stacked with one or more polynucleotides encoding a desirable trait such as a polynucleotide that confers, for example, insect, disease or herbicide resistance or other desirable agronomic traits of interest including, but not limited to, traits associated with high oil content; increased digestibility; balanced amino acid content; and/or high energy content. Such traits may refer to properties of both seed and non-seed plant tissues, or to food or feed prepared from plants or seeds having such traits.

Example polynucleotides of interest include_the following events: Btl1 (see U.S. Pat. No. 6,114,608), MIR604 (see U.S. Pat. No. 8,884,102), 5307 (see U.S. Pat. No. 10,428,393), MZIR098 (see US Patent Application No. US20200190533), TC1507 (see U.S. Pat. No. 7,288,643), DAS-59122-7 (see U.S. Pat. No. 7,323,556), MON810 (see U.S. Pat. No. 6,713,259), MON863 (see U.S. Pat. No. 7,705,216), MON89034 (see U.S. Pat. No. 8,062,840), MON88017 (see U.S. Pat. No. 9,556,492), DP-4114 (see U.S. Pat. No. 9,725,772), MON87411 (see U.S. Pat. No. 9,441,240), DP-032218-9 (see US Patent Application No. US2015361447), DP-033121-3 (see US Patent Application No. US2015361446), DP-023211-2 (see PCT Publication No. WO2019209700), MON95379 (see US Patent Application No. US2020032289), DBN9936 (see PCT Publication No. WO2016173361), DBN9501 (see PCT Publication No. WO20207125), GH5112E-117C (see PCT Publication No. WO17/088480), LP007-1 (see Chinese Patent Application No. CN112852801), LP007-2 (Chinese Patent Application No. CN112831584), LP007-3 (Chinese Patent Application No. CN112877454), LP007-4 (Chinese Patent Application No. CN112831585), LP007-5 (Chinese Patent Application No. CN113151534), LP007-6 (Chinese Patent Application No. CN113151533), LP007-7 (Chinese Patent Application No. CN112852991), LP007-8 (CN113980958), Ruifeng8, ND207, Ruifeng125 (see Chinese Patent Application No. CN105017391), GA21 (see PCT Publication No. WO98/44140), NK603 (see U.S. Pat. No. 6,825,400), DAS40278 (see PCT Publication No. WO2011/022469), DBN9858 (see PCT Publication No. WO2016173508), MON87429 (see PCT Publication No. WO19/152316), LW2-2 (see Chinese Patent Application No. CN113278721), T25 (see USDA/APHIS Petition 94-357-01 for Determination of Nonregulated Status for Glufosinate Resistant Corn Transformation Events T14 and T25, June 1995), or the 3272 event (see U.S. Pat. No. 7,635,799).

As used herein, gene or trait “stacking” is combining desired genes or traits into one transgenic plant line. As one approach, plant breeders stack transgenic traits by making crosses between parents that each have a desired trait and then identifying offspring that have both of these desired traits (so-called “breeding stacks”). Another way to stack genes is by transferring two or more genes into the cell nucleus of a plant at the same time during transformation. Another way to stack genes is by re-transforming a transgenic plant with another gene of interest. For example, gene stacking can be used to combine two different insect resistance traits, an insect resistance trait and a disease resistance trait, or a herbicide resistance trait (such as, for example, Btl1). The use of a selectable marker in addition to a gene of interest would also be considered gene stacking.

In some embodiments, a nucleic acid molecule or vector of the disclosure can include an additional coding sequence for one or more polypeptides or double stranded RNA molecules (dsRNA) of interest for agronomic traits that primarily are of benefit to a seed company, grower or grain processor. A polypeptide of interest can be any polypeptide encoded by a nucleotide sequence of interest. Non-limiting examples of polypeptides of interest that are suitable for production in plants include those resulting in agronomically important traits such as herbicide resistance (also sometimes referred to as “herbicide tolerance”), virus resistance, bacterial pathogen resistance, insect resistance, nematode resistance, or fungal resistance. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, relative maturity group, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956. In some embodiments, the expression of the polypeptide in the maize plants results in a different insect resistance than the one conferred by the MIR162 event. In such instances, stacking this resistance trait with the MIR162 event, by expressing the additional polypeptide in a MIR162 plant, can result in maize plants protected from insect feeding damage to a greater degree than the insect resistance traits conferred by the MIR162 event alone.

Polynucleotides conferring resistance/tolerance to an herbicide that inhibits the growing point or meristem, such as an imidazalinone or a sulfonylurea can also be suitable in some embodiments. Exemplary polynucleotides in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a mutant glutamine synthetase (GS) resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

Polypeptides encoded by nucleotides sequences conferring resistance to glyphosate are also suitable for the disclosure. See, e.g., U.S. Pat. Nos. 4,940,835 and 4,769,061. 5,554,798 discloses transgenic glyphosate resistant maize plants, which resistance is conferred by an altered 5-enolpyruvyl-3-phosphoshikimate synthase (EPSPS) gene.

Polynucleotides coding for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268, and 5,561,236.

Other suitable polynucleotides include those coding for resistance to herbicides that inhibit photosynthesis, such as a triazine and a benzonitrile (nitrilase) See, U.S. Pat. No. 4,810,648. Additional suitable polynucleotides coding for herbicide resistance include those coding for resistance to 2,2-dichloropropionic acid, sethoxydim, haloxyfop, imidazolinone herbicides, sulfonylurea herbicides, triazolopyrimidine herbicides, s-triazine herbicides and bromoxynil. Also suitable are polynucleotides conferring resistance to a protox enzyme, or that provide enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Additional suitable polynucleotides include those coding for insecticidal polypeptides. These polypeptides may be produced in amounts sufficient to control, for example, insect pests (i.e., insect controlling amounts). It is recognized that the amount of production of an insecticidal polypeptide in a plant necessary to control insects or other pests may vary depending upon the cultivar, type of pest, environmental factors and the like. Polynucleotides useful for additional insect or pest resistance include, for example, those that encode toxins identified in Bacillus organisms. Polynucleotides comprising nucleotide sequences encoding Bacillus thuringiensis (Bt) Cry proteins from several subspecies have been cloned and recombinant clones have been found to be toxic to lepidopteran, dipteran and/or coleopteran insect larvae. Examples of such Bt insecticidal proteins include the Cry proteins such as Cry1Aa, Cry1Ab, Cry1Ac, Cry1B, Cry1C, Cry1D, Cry1Ea, Cry1Fa, Cry3A, Cry9A, Cry9B, Cry9C, and the like, as well as vegetative insecticidal proteins such as Vip1, Vip2, Vip3, and the like. A full list of Bt-derived proteins can be found on the worldwide web at Bacillus thuringiensis Toxin Nomenclature Database maintained by the University of Sussex (see also, Crickmore et al. (1998) Microbiol. Mol. Biol. Rev. 62:807-813).

In embodiments, an additional polypeptide is an insecticidal polypeptide derived from a non-Bt source, including without limitation, an alpha-amylase, a peroxidase, a cholesterol oxidase, a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus spp. (such as B. laterosporous) insecticidal protein, a Lysinibacillus spp. (such as L. sphearicus) insecticidal protein, a Chromobacterium spp. (such as C. subtsugae or C. piscinae) insecticidal protein, a Yersinia spp. (such as Y. entomophaga) insecticidal protein, a Paenibacillus spp. (such as P. propylaea) insecticidal protein, a Clostridium spp. (such as C. bifermentans) insecticidal protein, a Pseudomonas spp. (such as P. fluorescens) and a lignin.

Polypeptides that are suitable for production in plants further include those that improve or otherwise facilitate the conversion of harvested plants or plant parts into a commercially useful product, including, for example, increased or altered carbohydrate content or distribution, improved fermentation properties, increased oil content, increased protein content, modified oil profile, improved digestibility, and increased nutraceutical content, e.g., increased phytosterol content, increased tocopherol content, increased stanol content or increased vitamin content. Polypeptides of interest also include, for example, those resulting in or contributing to a reduced content of an unwanted component in a harvested crop, e.g., phytic acid, or sugar degrading enzymes. By “resulting in” or “contributing to” is intended that the polypeptide of interest can directly or indirectly contribute to the existence of a trait of interest (e.g., increasing cellulose degradation by the use of a heterologous cellulase enzyme).

In some embodiments, the polypeptide contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced. Heterologous production of xylanases in plant cells also can facilitate lignocellulosic conversion to fermentable sugars in industrial processing.

Numerous xylanases from fungal and bacterial microorganisms have been identified and characterized (see, e.g., U.S. Pat. No. 5,437,992; Coughlin et al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol. 14:566; Torronen et al. (1992) Bio/Technology 10:1461; and Xu et al. (1998) Appl. Microbiol. Biotechnol. 49:718).

In other embodiments, a polypeptide useful for the disclosure can be a polysaccharide degrading enzyme. Plants of this disclosure producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme and other glucoamylases.

Polysaccharide-degrading enzymes include: starch degrading enzymes such as α-amylases (EC 3.2.1.1), glucuronidases (E.C. 3.2.1.131); exo-1,4-α-D glucanases such as amyloglucosidases and glucoamylase (EC 3.2.1.3), β-amylases (EC 3.2.1.2), α-glucosidases (EC 3.2.1.20), and other exo-amylases; starch debranching enzymes, such as a) isoamylase (EC 3.2.1.68), pullulanase (EC 3.2.1.41), and the like; b) cellulases such as exo-1,4-3-cellobiohydrolase (EC 3.2.1.91), exo-1,3-β-D-glucanase (EC 3.2.1.39), β-glucosidase (EC 3.2.1.21); c) L-arabinases, such as endo-1,5-α-L-arabinase (EC 3.2.1.99), α-arabinosidases (EC 3.2.1.55) and the like; d) galactanases such as endo-1,4-β-D-galactanase (EC 3.2.1.89), endo-1,3-β-D-galactanase (EC 3.2.1.90), α-galactosidase (EC 3.2.1.22), β-galactosidase (EC 3.2.1.23) and the like; e) mannanases, such as endo-1,4-β-D-mannanase (EC 3.2.1.78), 0-mannosidase (EC 3.2.1.25), α-mannosidase (EC 3.2.1.24) and the like; f) xylanases, such as endo-1,4-β-xylanase (EC 3.2.1.8), β-D-xylosidase (EC 3.2.1.37), 1,3-β-D-xylanase, and the like; and g) other enzymes such as α-L-fucosidase (EC 3.2.1.51), α-L-rhamnosidase (EC 3.2.1.40), levanase (EC 3.2.1.65), inulanase (EC 3.2.1.7), and the like. In one embodiment, the α-amylase is the synthetic α-amylase, Amy797E, described is U.S. Pat. No. 8,093,453, herein incorporated by reference in its entirety.

Further enzymes which may be used with the disclosure include proteases, such as fungal and bacterial proteases. Fungal proteases include, but are not limited to, those obtained from Aspergillus, Trichoderma, Mucor and Rhizopus, such as A. niger, A. awamori, A. oryzae and M. miehei. In some embodiments, the polypeptides of this disclosure can be cellobiohydrolase (CBH) enzymes (EC 3.2.1.91). In one embodiment, the cellobiohydrolase enzyme can be CBH1 or CBH2.

Other enzymes useful with the disclosure include, but are not limited to, hemicellulases, such as mannases and arabinofuranosidases (EC 3.2.1.55); ligninases; lipases (e.g., E.C. 3.1.1.3), glucose oxidases, pectinases, xylanases, transglucosidases, alpha 1,6 glucosidases (e.g., E.C. 3.2.1.20); esterases such as ferulic acid esterase (EC 3.1.1.73) and acetyl xylan esterases (EC 3.1.1.72); and cutinases (e.g. E.C. 3.1.1.74).

Double stranded RNA molecules useful with the disclosure include but are not limited to those that suppress target insect genes. As used herein the words “gene suppression”, when taken together, are intended to refer to any of the well-known methods for reducing the levels of protein produced as a result of gene transcription to mRNA and subsequent translation of the mRNA. Gene suppression is also intended to mean the reduction of protein expression from a gene or a coding sequence including posttranscriptional gene suppression and transcriptional suppression. Posttranscriptional gene suppression is mediated by the homology between of all or a part of a mRNA transcribed from a gene or coding sequence targeted for suppression and the corresponding double stranded RNA used for suppression and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a dsRNA to effect what is called RNA interference (RNAi). Transcriptional suppression is mediated by the presence in the cell of a dsRNA, a gene suppression agent, exhibiting substantial sequence identity to a promoter DNA sequence or the complement thereof to effect what is referred to as promoter trans suppression. Gene suppression may be effective against a native plant gene associated with a trait, e.g., to provide plants with reduced levels of a protein encoded by the native gene or with enhanced or reduced levels of an affected metabolite. Gene suppression can also be effective against target genes in plant pests that may ingest or contact plant material containing gene suppression agents, specifically designed to inhibit or suppress the expression of one or more homologous or complementary sequences in the cells of the pest. Such genes targeted for suppression can encode an essential protein, the predicted function of which is selected from the group consisting of muscle formation, juvenile hormone formation, juvenile hormone regulation, ion regulation and transport, digestive enzyme synthesis, maintenance of cell membrane potential, amino acid biosynthesis, amino acid degradation, sperm formation, pheromone synthesis, pheromone sensing, antennae formation, wing formation, leg formation, development and differentiation, egg formation, larval maturation, digestive enzyme formation, hemolymph synthesis, hemolymph maintenance, neurotransmission, cell division, energy metabolism, respiration, and apoptosis.

### VI. Plants, Plant Cells and Plant Parts

Maize plants used in the methods and compositions are plants from Zea mays L. subsp. Mays. In some embodiments, the maize plant or plant part is from the group Zea mays L. subsp. Mays identata, sometimes referred to as dent corn. In some embodiments, the maize plant or plant part is from the group Zea mays L. subsp. Mays indurata, sometimes referred to as flint corn. In some embodiments, the maize plant or plant part is from the group Zea mays L. subsp. Mays saccharata, sometimes referred to as sweet corn. In some embodiments, the maize plant or plant part is from the group Zea mays L. subsp. Mays amylacea, sometimes referred to as flour corn. In some embodiments, the maize plant or plant part is from the group Zea mays L. subsp. Mays EVERTS, sometimes referred to as popcorn. Maize plants that can be identified selected and/or produced with methods and compositions of the present invention include hybrids, inbreds, partial inbreds, members of defined populations and members of undefined populations. In some embodiments, the maize plant is an elite maize line. In some embodiments, the elite line is one of NP2222, NP2660, NP2276, NP2391, NP2460, or ID3461 or one of the lines disclosed in Table 5.

In some embodiments, a plant cell, seed, or plant part or harvest product can be obtained from a plant produced as above and the plant cell, seed, or plant part can be screened using methods disclosed above for the evidence of stable incorporation of the polynucleotide. Examples of plant parts include, but are not limited to, single cells and tissues from pollen, ovules, zygotes, leaves, embryos, roots, root tips, anthers, flowers, flower parts, fruits, stems, shoots, cuttings, and seeds; as well as pollen, ovules, egg cells, zygotes, leaves, embryos, roots, root tips, anthers, flowers, flower parts, fruits, stems, shoots, cuttings, scions, rootstocks, seeds, protoplasts, calli, and the like.

In some embodiments, plant products can be harvested from the plant disclosed above and processed to produce processed products, such as meal, oil, plant extract, starch, fermentation products, digestion products, and the like. These processed products are also within the scope of this invention if they comprise a polynucleotide or polypeptide or variant thereof disclosed herein. In another example, this disclosure also provides a corn meal. In another example, this disclosure also provides a method of providing a corn meal by crushing oilseed of any of the plants provided herein.

### VII. Breeding Program and Seed Production Program

In some embodiments, a MIR162 maize plant can be bred by crossing a first parental maize plant that is a transgenic MIR162 maize plant comprising one or more fertile QTLs and/or TE as described above and a second parental maize, thereby producing a plurality of first progeny plants. In some embodiments, the second parental maize plant does not comprise MIR162 event. The method further comprises selecting a first progeny plant that comprises the MIR162 event and the one or more fertile QTLs.

In some embodiments, the method further comprises selfing the first progeny plant, thereby producing a plurality of second progeny plants; and then selecting from the second progeny plants that comprise both the MIR162 event and the fertile QTL(s) and/or TE.

In some embodiments, the first progeny plant is repeatedly crossed back to one of its parents through a process referred to herein as “backcrossing”. In some embodiments, the progeny is backcrossed for at least two generations to the maize parental plant (e.g., the second maize parental plant). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. (1995) Marker-assisted Backcrossing: A Practical Example, in Techniques et Utilisations des Marqueurs Moleculaires Les Colloques, Vol. 72, pp. 45-56; and Openshaw et al., (1994) Marker-assisted Selection in Backcross Breeding, in Proceedings of the Symposium “Analysis of Molecular Marker Data,” Joint Plant Breeding Symposia Series, American Society for Horticultural Science/Crop Science of America, Corvallis, Oregon, pp. 41-43. The initial cross gives rise to the F1 generation. The term “BC1” typically refers to the second use of the recurrent parent, “BC2” refers to the third use of the recurrent parent, and so on. The progeny plants may then be genotyped to confirm the presence of the MIR162 event and the fertile QTLs.

### Exemplary Embodiments

Embodiment 1 is a method of producing a MIR162 plant with increased fertility, said method comprising: selecting a first maize plant comprising one or more fertile QTLs, wherein the one or more fertile QTLs comprise one or more marker loci of SEQ ID NOs: 1-223 or 255, wherein at least one marker locus of the one or more marker loci comprises a favorable allele at position number 100 as shown in Table 3 or Table 4 or Table 9, crossing the first maize plant with a second maize plant to produce a plurality of progeny plants, wherein the first maize plant and/or the second maize plant comprises maize event MIR162, and selecting from the plurality of progeny plants a selected progeny MIR162 plant comprising one or more fertile QTLs.

Embodiment 2 is a method of producing a MIR162 plant with increased fertility, said method comprising: selecting a first maize plant comprising one or more marker loci linked to and within 10 centimorgans (cM) of one or more fertile QTL, wherein the one or more fertile QTLs are selected from the group consisting of a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3; a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4. SNPs significantly associated with MIR162 event conversion success; a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4; a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4; a QTL located on chromosome 8 at position 14,787,385 comprising the marker locus of SEQ ID NO: 255, wherein the marker locus comprises a favorable allele at position number 100 as shown in Table 9; and a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4, and crossing the first maize plant with a second maize plant to produce a plurality of progeny plants, wherein the first maize plant and/or the second maize plant comprises maize MIR162 event; and selecting from the plurality of progeny plants a selected progeny MIR162 plant comprising the one or more fertile QTL using the one or more marker loci linked to and within 10 centimorgans (cM) of the one or more fertile QTL.

Embodiment 3 is a method of producing a MIR162 plant with increased fertility, said method comprising: selecting a first maize plant comprising one or more fertile QTLs, wherein the one or more fertile QTLs comprise one or more marker loci of SEQ ID NOs: 1-223 or 255, wherein at least one marker locus of the one or more marker loci comprises a favorable allele at position number 100 as shown in Table 3 or Table 4 or Table 9, and introducing a MIR162 event into the selected maize plant.

Embodiment 4 is a method of producing a MIR162 plant with increased fertility, said method comprising: selecting a first maize plant comprising one or more marker loci linked to and within 10 centimorgans (cM) of one or more fertile QTL, wherein the one or more fertile QTLs are selected from the group consisting of a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3; a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4; a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4; a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4; a QTL located on chromosome 8 at position 14,787,385 comprising the marker locus of SEQ ID NO: 255, wherein the marker locus comprises a favorable allele at position number 100 as shown in Table 9; and a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4, and introducing a MIR162 event into the selected maize plant.

Embodiment 5 is the method of any one of embodiments 1-4, wherein the MIR162 event is introduced to the selected maize plant by transforming or breeding in a Vip3A coding sequence into the selected maize plant.

Embodiment 6 is the method of any one of embodiments 1-4, wherein the method further comprises producing an inbred MIR162 plant from the selected maize plant containing MIR162.

Embodiment 7 is the method of any one of embodiments 1-6, wherein the first maize plant comprises a QTL associated with increased fertility and the QTL comprises SEQ ID NO: 2, with a C at the position number 100.

Embodiment 8 is the method of any one of embodiments 1-6, wherein the one or more fertile QTLs comprise one or more of: SEQ ID NO: 1 with a G at position number 100, SEQ ID NO: 2 with a C at position number 100, SEQ ID NO: 3 with a G at position number 100, SEQ ID NO: 4 with an A at position number 100, or SEQ ID NO: 5 with a C at position number 100.

Embodiment 9 is the method of any one of embodiments 1-6, wherein the one or more fertile QTLs comprise SEQ ID NO: 2, 52, 88, 139, and 94, wherein position number 100 of each sequence, respectively and in combination, is the one of the following: (i) C, A, A, T, and T; (ii) C, A, A, C, and T; (iii) C, A, G, C, and T; (iv) C, G, A, C, and T; (v) C, G, G, C, and T; (vi) C, G, G, C, and C; (vii) A, A, A, T, and T; (viii) A, A, G, T, and T; (ix) A, A, A, C, and T; (x) A, G, A, T, and T; (xi) A, G, A, C, and T; or (xii) A, A, G, C, and T.

Embodiment 10 is a method of producing a MIR162 plant with increased fertility, said method comprising: selecting a first maize plant comprising a transposon element (TE) comprising SEQ ID NO: 232, 233, or a variant thereof that is at least 90% identical to SEQ ID NO: 232 or 233, crossing the first maize plant with a second maize plant to produce a plurality of progeny plants, wherein the first maize plant and/or the second maize plant comprises maize MIR162 event, and selecting from the plurality of progeny plants a selected progeny MIR162 plant comprising the transposon element.

Embodiment 11 is the method of example(s) 10, wherein the transposon element is on chromosome 5 130436801-130439201 in B73_ver5.

Embodiment 12 is the method of any one of embodiments 1-11, wherein the one or more fertile QTLs or the TE is introduced to the first maize plant by genome editing or by breeding.

Embodiment 13 is the method of example(s) 12, wherein the genome editing is performed using a site-directed nuclease selected from the group consisting of Cas9 nuclease, Cpfnucleas (Cas12a), meganucleases (MNs), zinc-finger nucleases, (ZFNs), transcription-activator like effector nucleases (TALENs), dCas9-Fokl, dCpf1-FokI, chimeric Cas9-cytidine deaminase, chimeric Cas9-adenine deaminase, chimeric FENI-FokI, MegaTALs, a nickase Cas9 (nCas9), chimeric dCas9 non-Fokl nuclease, dCpf1 non-Fokl nuclease, chimeric Cpf1-cytidine deaminase, and Cpf1-adenine deaminase.

Embodiment 14 is the method of any one of embodiments 1-13, wherein the first maize plant and/or the second maize plant is an elite maize line.

Embodiment 15 is the method of example(s) 1, wherein the increased fertility is increased male fertility.

Embodiment 16 is the method of any one of embodiments 1-15, wherein the method further comprises using the selected progeny MIR162 plant as a pollinator in a second cross with itself or a third maize plant.

Embodiment 17 is the method of example(s) 16, wherein the third maize plant expresses a polynucleotide or polypeptide of interest.

Embodiment 18 is the method of example(s) 17, wherein the polynucleotide or polypeptide of interest confers insect resistance, disease resistance, herbicide resistance, high oil content, increased digestibility; balanced amino acid content, and/or high energy content.

Embodiment 19 is the method of example(s) 16, wherein the second cross produces increased seed production as compared to a control cross.

Embodiment 20 is a method of producing a plant compatible with a MIR162 event, said method comprising: selecting a first maize plant comprising a transposon element (TE) comprising SEQ ID NO: 232, 233, or a variant thereof that is at least 90% identical to SEQ ID NO: 232 or 233, crossing the first maize plant with a second maize plant to produce a plurality of progeny plants, and selecting from the plurality of progeny plants a selected progeny plant comprising the transposon element.

Embodiment 21 is the method of example(s) 20, wherein the method further comprises crossing the selected progeny plant comprising the transposon element with a second maize plant comprising maize MIR162 event.

Embodiment 22 is a breeding program comprising the method of any one of embodiments 1-21.

Embodiment 23 is a MIR162 plant produced according to any one of embodiments 1-19.

Embodiment 24 is a MIR162 plant comprising one or more fertile QTLs, wherein the one or more fertile QTLs comprise one or more marker loci of SEQ ID NOs: 1-223 or 255, wherein at least one marker locus of the one or more marker loci comprises a favorable allele at position number 100 as shown in Table 3 or Table 4 or Table 9.

Embodiment 25 is a MIR162 plant comprising one or more marker loci linked to and within 10 centimorgans (cM) of one or more fertile QTL, wherein the one or more fertile QTLs are selected from the group consisting of a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3; a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4; a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4; a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4; a QTL located on chromosome 8 at position 14,787,385 comprising the marker locus of SEQ ID NO: 255, wherein the marker locus comprises a favorable allele at position number 100 as shown in Table 9; and a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4.

Embodiment 26 is the MIR162 plant of example(s) 24 or 25, wherein the one or more fertile QTLs comprise SEQ ID NO: 2, with a C at the position number 100.

Embodiment 27 is the MIR162 plant of example(s) 24 or 25, wherein the one or more fertile QTLs comprise one or more of: SEQ ID NO: 1, with a G at position number 100, SEQ ID NO: 2, with a C at position number 100, SEQ ID NO: 3, with a G at position number 100, SEQ ID NO: 4, with an A at position number 100, or SEQ ID NO: 5, with a C at position number 100.

Embodiment 28 is the method of example(s) 24 or 25, wherein one or more fertile QTLs comprise SEQ ID NO: 2, 52, 88, 139, and 94, wherein position number 100 of each sequence, respectively and in combination, is the one of the following: (i) C, A, A, T, and T; (ii) C, A, A, C, and T; (iii) C, A, G, C, and T; (iv) C, G, A, C, and T; (v) C, G, G, C, and T; (vi) C, G, G, C, and C; (vii) A, A, A, T, and T; (viii) A, A, G, T, and T; (ix) A, A, A, C, and T; (x) A, G, A, T, and T; (xi) A, G, A, C, and T; or (xii) A, A, G, C, and T.

Embodiment 29 is a MIR162 plant comprising a transposon element (TE) comprising SEQ ID NO: 232, 233, or a variant thereof that is at least 90% identical to SEQ ID NO: 232 or 233.

Embodiment 30 is the MIR162 plant of example(s) 29 wherein the transposon element is on chromosome 5 130436801-130439201 in B73 ver5.

Embodiment 31 is the MIR162 plant of example(s) 23-31, wherein the MIR162 plant is an inbred MIR162 plant.

Embodiment 32 is a plant cell, seed, or plant part derived from the MIR162 plant of any one of embodiments 24-33.

Embodiment 33 is a harvested product derived from the MIR162 plant of any one of embodiments 24-33 or the plant cell, seed, or plant part of example(s) 32.

Embodiment 34 is a kit for genotyping a plant wherein the kit comprises reagents for detecting the presence of one or more marker loci of SEQ ID Nos: 1-223 or 255; one or more marker loci linked to and within 10 centimorgans (cM) of one or more fertile QTL, wherein the one or more fertile QTLs are selected from the group consisting of a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3; a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4; a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4; a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4; a QTL located on chromosome 8 at position 14,787,385 comprising the marker locus of SEQ ID NO: 255, wherein the marker locus comprises a favorable allele at position number 100 as shown in Table 9; and a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4; and/or a transposon element (TE) of SEQ ID NO: 232, 233, or a variant thereof that is at least 90% identical to SEQ ID NO: 232 or 233.

Embodiment 35 is the kit of example(s) 34, wherein reagents comprise one or more of the following: primers for sequencing the one or more marker loci and/or the TE, primers for amplifying the one or more marker loci and/or the TE, or a oligonucleotide probe that is complementary to one or more marker loci.

Embodiment 36 is a method of detecting one or more fertile QTLs in an MIR162 plant, the method comprising genotyping the plant at one or more marker loci linked to one or more fertile QTL, wherein the fertile QTL comprises one or more marker loci of SEQ ID Nos 1-223 or 255, wherein at least one marker locus of the one or more marker loci comprises a favorable allele at position number 100 as shown in Table 3 or Table 4 or Table 9.

Embodiment 37 is a method of detecting one or more marker loci linked to and within 10 centimorgans (cM) of one or more fertile QTL, the method comprising genotyping one or more fertile QTLs from the group consisting of a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3; a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4; a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4; a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4; a QTL located on chromosome 8 at position 14,787,385 comprising the marker locus of SEQ ID NO: 255, wherein the marker locus comprises a favorable allele at position number 100 as shown in Table 9; and a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4.

Embodiment 38 is the method of example(s) 36 or 37, wherein the one or more fertile QTLs comprise one or more of: SEQ ID NO: 1 with a G at position number 100, SEQ ID NO: 2 with a C at position number 100, SEQ ID NO: 3 with a G at position number 100, SEQ ID NO: 4 with an A at position number 100, or SEQ ID NO: 5 with a C at position number 100.

Embodiment 39 is the method of example(s) 36-38 wherein the one or more fertile QTLs comprise SEQ ID NO: 2, 52, 88, 139, and 94, wherein position number 100 of each sequence, respectively and in combination, is the one of the following: (i) C, A, A, T, and T; (ii) C, A, A, C, and T; (iii) C, A, G, C, and T; (iv) C, G, A, C, and T; (v) C, G, G, C, and T; (vi) C, G, G, C, and C; (vii) A, A, A, T, and T; (viii) A, A, G, T, and T; (ix) A, A, A, C, and T; (x) A, G, A, T, and T; (xi) A, G, A, C, and T; or (xii) A, A, G, C, and T.

Embodiment 40 is the method of any one of embodiments 36-39, wherein the genotyping is performed by sequencing, polymerase chain reaction (PCR), probe hybridization, or single base extension.

The examples presented below are intended to illustrate potential and specific implementations of the disclosure. It can be appreciated that the examples are intended primarily for purposes of illustration of the disclosure for those skilled in the art. There may be variations to these diagrams, or the operations described herein without departing from the spirit of the disclosure. For instance, in certain cases, method steps or operations may be performed or executed in differing order, or operations may be added, deleted or modified.

### EXAMPLES

### Example 1. Chromosome 5 Locus Identification

The MIR162 event is known to be associated with a reduced male fertility phenotype in certain inbred genetic backgrounds, which is thought to be attributed to reduced pollen shed. In a prior study, a first approach was to use a genome-wide association study (GWAS) (Yu et al. 2006) to identify genomic loci associated with the MIR162 inbred pollen shed phenotype. However, due to complicated genetic architecture, even with tremendous efforts in the GWAS approach, a portion of the variation of the MIR162 male fertility phenotype observed in certain maize backgrounds remained unexplained. In a second study, a bi-parental QTL mapping approach was used. Loci from the second study are disclosed, e.g., in U.S. Pat. No. 10,214,784. However, these loci did not contribute to an understanding of the full variation of the MIR162-associated male fertility phenotype observed in certain maize backgrounds.

In the current study, a genomic random forest (GRAF) prediction analytics model was used by integrating random forest into genomic prediction. Genotype and phenotype data of 338 inbred lines that went through the trait introgression program from 2017 to 2020 in maize was used (Table 2). Phenotypic data was classified as a binary trait, as either “failure” or “success” for each MIR162 conversion project. A MIR162 conversion project typically involved the initial crossing of an elite inbred line with a MIR162-containing donor line followed by repeated backcrossing to the elite inbred line to introgress the MIR162 event. Each inbred was evaluated based on 1) if they met the minimum seed quantity (e.g., producing at least 130 seeds per conversion event) and number of versions of conversion events resulted from individual backcrosses to be considered a successful trait introgression and 2) if it did not meet the key performance indicators (KPIs) in 1), were there other factors besides MIR162 that contributed to the failure. If the answer to the first question was “Yes”, it was defined as “success”, and if the answer to both questions was “No”, it was defined as a “failure”. If the answer to the question 2 was “Yes”, it was eliminated from the training data set. Each conversion was also followed through Version Testing (VT) and Seed Production Research (SPR) and if it failed a version test because of low pollen count the previous rating of “success” was updated to “failure”. In the context of this analysis, a version is a distinct instance of a finished conversion that derives from an individual backcross plant. Typically, backcrossing is performed until a certain % recurrent parent recovery (% RP) (e.g., >95% RP) is achieved (typically 2-4 backcrossed) and then self pollination of backcrossed plants (e.g., BC2, BC3 or BC4) is performed to start fixing the traits. 3-4 versions of each conversion were analyzed. Each version was derived from a unique BC plant that was selfed twice to create a BxF3 converted line, e.g., 4 B3F3 versions handed off and each one derived back to a different BC3 plant that was selfed. The minimum number of versions that was considered “successful” is 2 versions. The minimum seed quantity per version that is considered successful was 130 seeds per version. Over four years, the MIR162 failure rate averaged 0.43 with a range from 0.41 to 0.47. This failed conversion of MIR162 is thought to attributed to a decrease in male fertility in inbreds.

In the context of this analysis, the failure rate refers to the number of elite inbreds for which a MIR162 conversion was attempted and did not succeed (i.e. the plants remained infertile or had reduced fertility) out of the total number MIR162 conversions that were attempted in a MIR162 conversion project. For example, in one experiment, it was attempted to convert 123 inbreds as described herein and 58 of them were not successful. Thus, the term “failure rate” in this context refers to the number of MIR162 conversion attempts (e.g., elite inbred or recurrent parents) that are infertile out the total number of MIR162 conversions s produced from a predetermined number of MIR162 projects. Conversely, the term “success rate” refers to the number of MIR162 conversions that are fertile (e.g., successful trait stacking) over the total number of MIR162 conversions produced from a predetermined number of MIR162 projects. In some embodiments, the predetermined number is at least 10, at least 20, at least 30, at least 40, at least 50, at least 60, at least 70, at least 80, at least 90, at least 100, at least 110, or at least 120. The sum of the success rate and the failure rate for particular event equals 1 or 100%.

A total of 5,030 SNPs were used to genotype each inbred line before MIR162 conversion, and the genotypic data from these inbreds were used for subsequent analysis. The genotypes from each untraited inbred line were used for the training and prediction of the GRAF model.

TABLE 2
Binary phenotypic decision data for MIR162 event conversion

| | | | | Failure | Success |
|---|---|---|---|---|---|
| Year | Failure | Success | Total | rate | rate |
| 2017 | 58 | 65 | 123 | 0.47 | 0.53 |
| 2018 | 35 | 46 | 81 | 0.43 | 0.57 |
| 2019 | 30 | 40 | 70 | 0.43 | 0.57 |
| 2020 | 26 | 38 | 64 | 0.41 | 0.59 |

The GRAF model was used to identify novel loci associated with MIR162 event conversion. One locus on Chromosome 5 was highly correlated with the conversion failure (see Table 3 below). Additional further analysis using the GRAF model also identified SNPs on chromosomes 2 and 8 as being highly correlated with MIR162 event conversion (see Table 9 below). In particular, the SNP at position 129259011 of Chromosome 5 was the most significantly correlated with the phenotype within the locus according to the GRAF model. These loci and/or the SNPs contained therein are considered to provide stronger predictive power than previous loci and SNPs disclosed in U.S. Pat. No. 10,214,784.

TABLE 3
Chromosome 5 SNPs significantly associated with MIR162 event conversion success

| | | | | | | No. of | No. of | |
|---|---|---|---|---|---|---|---|---|
| | | Position on | | | SEQ | plants with | successes with | |
| Marker | SNP Code | chromosome | Favorable | Unfavorable | ID | favorable | favorable | Success |
| locus # | ID | (bp) | Allele | Allele | NO | allele | allele | rate |
| 1 | 9_12627822 | 126407035 | G | A | 1 | 102 | 93 | 91% |
| 2 | 9_12627976 | 129259011 | C | A | 2 | 113 | 103 | 91% |
| 3 | 9_12627772 | 130949501 | G | A | 3 | 93 | 86 | 92% |
| 4 | 9_12627820 | 131470622 | A | G | 4 | 165 | 134 | 81% |
| 5 | 9_12627920 | 137510449 | C | A | 5 | 147 | 119 | 81% |

TABLE 9
Chromosome 2 and 8 SNPs significantly associated
with MIR162 event conversion success

| | | | | | SEQ ID NO | |
|---|---|---|---|---|---|---|
| | | Position on | | | of region, N = | |
| | | chromosome | Favorable | Unfavorable | position of | Importance |
| SNP Code | Chrom. | (bp) | Allele | Allele | SNP in region | Index |
| 9_12661204 | 2 | 241,883,899 | C | T | 49 | 0.003506 |
| 9_12668711 | 2 | 243,907,360 | T | C | 53 | 0.008007 |
| 9_12648983 | 2 | 243,529,534 | A | G | 52 | 0.002730 |
| 9_12634054 | 8 | 14,787,385 | G | A | 255 | 0.005206 |

The importance index in Table 9 is a measure of the relative importance of each SNP in predicting the outcome of the model. It is calculated by measuring the decrease in the model's accuracy when the values of a particular feature are randomly permuted. The higher the decrease in accuracy, the more important the feature is considered to be in the model. This index can be used to identify the most important genomic markers in the model and to gain insights into the relationships between the markers and the outcome of MIR162 event conversion.

SEQ ID NO: 255:
TCTGTAAAACAAACAGCCATTTGATCCATTCAGCCTGATTTTTCGTATAG
GGTCAAAAAACTTGGTAACTTCAAAGCAAGGCACTAAATGTTCACATGAA
CAAAACAAAGGGCAGACCCAGCTCCCACATGAATGTGGGGTCTTTCCCCC
ACAAATGCGGAAAGGAAAATCCAGCATGTAAATTCATCCAAGCAACAGAA
ACAAAAACTCGGCCNGGGAAGGAAAGACCGCCCTCCCGGTATTCTATTAA
GAAGAGACCGAAACATGGTCCCGGCCGAAAAAATCCCCGAACCCTAGCCC
CCCATCACTAGTTGGCCGACATCGCGCACTCTGCAAATGCCCAGCCGGAG
GGTGGGGTGCATGACATAACCCGAGGGCGGGCGGGGCACAACGAAGGGAT
TTTTTAACCAAGCCCGAAATTCGCCCCCAAGGGGGATCGAACCCGGGACC
TGGAGGTGCTACTTGGAAGCTTTAACCATTACGCTAAAGGCCCTTTCGCA
GCGACAGAAACACACATAAGTTGATTCCTAAACTAAAGCTGTTACAGAAA
AACAGTAATGTCCGAACTCCGGATATGTAGATTATCTAACAGAACTGATC
ACCAGAAGCAAAGGGCCTGTTCAGTTTGGGTTTCAGCTGCTG

### Example 2. Other Non-Chromosome 5 Locus Identification

While lines that did contain the favorable allele at the SNP at position 129259011 of Chromosome 5 had about an 80% success rate, lines that did not contain the favorable allele at the SNP at position 129259011 of Chromosome 5 had about a 50% success rate. This indicates that there are other genomic loci that correlate with successful MIR162 event conversion and could be used instead of or with the SNP on Chromosome 5. To discover possibly new, conditionally distinct loci, fertile individuals carrying the favorable allele at the Chromosome 5 locus were removed from the sample set and a new sample set of 411 lines was used for a new GWAS. Structure analysis was performed to detect and correct for false positives due to population relatedness using a Nei similarity kinship matrix. GWAS was performed using a precomputed K (kinship) similarity matrix. Loci were detected on Chromosomes, 2, 6, 7 and 9 having SNPs with a significant correlation to the conversion phenotype (see Table 4 below). In these loci, the following SNPs were the most significantly correlated with the conversion phenotype: Chromosome 2 at position 243529534, chromosome 6 at position 158572433, chromosome 7 at position 134444680, and chromosome 9 at position 8013654. The favorable alleles in Table 4 could be used to positively select varieties that are likely to have a successful MIR162 conversion and the unfavorable alleles could be used to eliminate varieties that are likely to have an unsuccessful MIR162 conversion. The loci and/or the SNPs contained therein in Table 4 and Table 3, e.g., in combinations of two, three, or more, were considered to be of stronger predictive power than previous loci and SNPs disclosed in U.S. Pat. No. 10,214,784.

TABLE 4
SNPs significantly associated with MIR162 event conversion success

| | | | | | SEQ ID | | |
|---|---|---|---|---|---|---|---|
| | | | | | NO of | | |
| | | | | | region, N = | | |
| | | Position on | | | position of | | |
| | | chromosome | Favorable | Unfavorable | SNP in | | |
| SNP Code | Chrom. | (bp) | Allele | Allele | region | locus.logp | Locus.logFDR |
| 9_12648195 | 2 | 220,204,265 | G | A | 6 | 5.021 | 3.138 |
| 9_12632559 | 2 | 221,013,701 | C | T | 7 | 6.124 | 3.576 |
| 9_12632435 | 2 | 221,014,747 | T | C | 8 | 6.655 | 3.858 |
| 9_12648293 | 2 | 221,449,222 | G | A | 9 | 3.491 | 2.347 |
| 9_12664455 | 2 | 221,455,842 | A | C | 10 | 3.572 | 2.390 |
| 9_12648345 | 2 | 221,919,777 | A | G | 11 | 3.498 | 2.352 |
| 9_12648219 | 2 | 222,495,398 | C | T | 12 | 4.180 | 2.748 |
| 9_12666490 | 2 | 223,402,380 | G | C | 13 | 4.432 | 2.903 |
| 9_12632229 | 2 | 223,802,023 | A | G | 14 | 3.813 | 2.535 |
| 9_12664513 | 2 | 223,802,087 | G | T | 15 | 3.536 | 2.371 |
| 9_12664515 | 2 | 223,802,247 | A | C | 16 | 3.748 | 2.510 |
| 9_12664900 | 2 | 226,345,787 | C | G | 17 | 5.271 | 3.223 |
| 9_12648503 | 2 | 226,692,234 | T | C | 18 | 4.989 | 3.122 |
| 9_12648401 | 2 | 226,695,985 | T | C | 19 | 4.698 | 2.964 |
| 9_12648558 | 2 | 227,456,844 | G | A | 20 | 4.575 | 2.937 |
| 9_12634276 | 2 | 231,641,173 | C | T | 21 | 3.348 | 2.258 |
| 9_67926582 | 2 | 231,642,040 | A | C | 22 | 3.348 | 2.258 |
| 9_67925517 | 2 | 232,178,347 | T | C | 23 | 4.592 | 2.951 |
| 9_12633459 | 2 | 232,517,703 | G | T | 24 | 3.340 | 2.253 |
| 9_12633460 | 2 | 232,517,799 | A | G | 25 | 3.067 | 2.086 |
| 9_12648580 | 2 | 233,658,276 | G | A | 26 | 3.115 | 2.118 |
| 9_12663433 | 2 | 234,534,902 | C | T | 27 | 4.880 | 3.043 |
| 9_12648499 | 2 | 235,051,089 | T | C | 28 | 3.507 | 2.356 |
| 9_12648579 | 2 | 235,056,628 | A | G | 29 | 3.301 | 2.229 |
| 9_12648578 | 2 | 235,060,777 | C | A | 30 | 3.229 | 2.181 |
| 9_12648513 | 2 | 235,062,868 | G | A | 31 | 3.301 | 2.229 |
| 9_12648508 | 2 | 235,078,329 | C | T | 32 | 4.160 | 2.735 |
| 9_12648480 | 2 | 235,788,869 | C | T | 33 | 3.832 | 2.547 |
| 9_12649090 | 2 | 236,271,580 | A | G | 34 | 4.671 | 2.964 |
| 9_12649084 | 2 | 236,785,733 | T | C | 35 | 3.622 | 2.420 |
| 9_12649078 | 2 | 238,193,674 | C | A | 36 | 4.532 | 2.932 |
| 9_12649102 | 2 | 238,199,546 | G | A | 37 | 4.436 | 2.904 |
| 9_12648900 | 2 | 239,006,852 | G | A | 38 | 3.961 | 2.614 |
| 9_12661882 | 2 | 239,866,487 | G | A | 39 | 7.376 | 4.425 |
| 9_12649068 | 2 | 239,873,039 | A | G | 40 | 6.426 | 3.773 |
| 9_12648965 | 2 | 239,888,276 | C | A | 41 | 7.071 | 4.178 |
| 9_12648884 | 2 | 239,888,529 | T | C | 42 | 5.383 | 3.273 |
| 9_12648951 | 2 | 239,889,178 | A | G | 43 | 5.360 | 3.271 |
| 9_12649058 | 2 | 240,012,142 | A | G | 44 | 5.377 | 3.273 |
| 9_12648966 | 2 | 240,013,229 | G | A | 45 | 5.904 | 3.505 |
| 9_12649265 | 2 | 240,149,427 | T | C | 46 | 7.995 | 4.976 |
| 9_12648956 | 2 | 240,320,463 | G | A | 47 | 6.118 | 3.576 |
| 9_12633664 | 2 | 241,695,303 | G | T | 48 | 3.022 | 2.056 |
| 9_12661204 | 2 | 241,883,899 | C | T | 49 | 9.148 | 5.829 |
| 9_12648947 | 2 | 242,416,915 | A | C | 50 | 3.552 | 2.378 |
| 9_12649030 | 2 | 242,908,554 | A | G | 51 | 3.230 | 2.181 |
| 9_12648983 | 2 | 243,529,534 | A | G | 52 | 10.262 | 6.466 |
| 9_12668711 | 2 | 243,907,360 | T | C | 53 | 8.863 | 5.669 |
| 9_12651808 | 2 | 244,109,662 | A | G | 54 | 9.256 | 5.829 |
| 9_12651616 | 2 | 244,411,717 | G | A | 55 | 5.126 | 3.152 |
| 9_12616880 | 6 | 146,526,712 | A | G | 56 | 4.654 | 2.964 |
| 9_12650485 | 6 | 146,533,637 | C | T | 57 | 4.654 | 2.964 |
| 9_12617069 | 6 | 146,533,970 | C | T | 58 | 4.566 | 2.934 |
| 9_12617018 | 6 | 146,680,665 | G | A | 59 | 6.147 | 3.576 |
| 9_12617021 | 6 | 146,905,334 | C | A | 60 | 4.665 | 2.964 |
| 9_12617045 | 6 | 146,908,985 | T | G | 61 | 4.479 | 2.927 |
| 9_12617017 | 6 | 146,914,894 | T | G | 62 | 4.479 | 2.927 |
| 9_12616935 | 6 | 146,914,981 | A | G | 63 | 5.054 | 3.143 |
| 9_12616906 | 6 | 146,915,382 | T | G | 64 | 4.479 | 2.927 |
| 9_12616934 | 6 | 146,918,564 | G | T | 65 | 5.159 | 3.176 |
| 9_12616944 | 6 | 146,987,289 | C | T | 66 | 4.770 | 2.978 |
| 9_12616847 | 6 | 146,989,136 | C | A | 67 | 4.770 | 2.978 |
| 9_12616914 | 6 | 147,234,110 | G | A | 68 | 4.717 | 2.964 |
| 9_12650386 | 6 | 147,780,305 | T | C | 69 | 4.908 | 3.057 |
| 9_236648968 | 6 | 147,949,285 | A | T | 70 | 5.117 | 3.152 |
| 9_12617047 | 6 | 147,951,329 | G | A | 71 | 4.630 | 2.964 |
| 9_12616909 | 6 | 148,095,701 | T | G | 72 | 4.632 | 2.964 |
| 9_12616940 | 6 | 148,383,482 | T | C | 73 | 4.446 | 2.910 |
| 9_12616933 | 6 | 148,485,229 | A | G | 74 | 4.446 | 2.910 |
| 9_12617065 | 6 | 148,604,164 | A | G | 75 | 4.363 | 2.868 |
| 9_12616929 | 6 | 148,856,952 | T | C | 76 | 3.187 | 2.160 |
| 9_12616988 | 6 | 148,929,841 | T | C | 77 | 5.207 | 3.207 |
| 9_12617059 | 6 | 149,159,790 | T | C | 78 | 4.932 | 3.075 |
| 9_12616832 | 6 | 149,401,839 | T | C | 79 | 4.298 | 2.818 |
| 9_12665583 | 6 | 149,410,520 | C | T | 80 | 4.599 | 2.952 |
| 9_12665585 | 6 | 149,410,749 | A | G | 81 | 4.599 | 2.952 |
| 9_12665581 | 6 | 149,410,930 | T | C | 82 | 4.787 | 2.982 |
| 9_12616861 | 6 | 149,716,866 | A | G | 83 | 5.236 | 3.218 |
| 9_12617016 | 6 | 149,717,734 | C | A | 84 | 5.438 | 3.273 |
| 9_12664078 | 6 | 150,034,051 | C | T | 85 | 3.122 | 2.121 |
| 9_12616936 | 6 | 150,034,828 | G | A | 86 | 6.506 | 3.788 |
| 9_12650391 | 6 | 150,161,054 | A | G | 87 | 3.283 | 2.223 |
| 9_12633481 | 6 | 158,572,433 | A | G | 88 | 8.172 | 5.075 |
| 9_12634011 | 6 | 168,005,927 | C | G | 89 | 3.700 | 2.474 |
| 9_12621641 | 7 | 130,409,997 | G | A | 90 | 3.001 | 2.046 |
| 9_12621766 | 7 | 131,823,521 | A | G | 91 | 3.769 | 2.523 |
| 9_12621604 | 7 | 132,495,308 | G | A | 92 | 3.237 | 2.186 |
| 9_12621720 | 7 | 132,795,329 | A | G | 93 | 4.464 | 2.916 |
| 9_12621982 | 7 | 134,444,680 | T | C | 94 | 5.873 | 3.505 |
| 9_12667237 | 7 | 137,483,036 | C | T | 95 | 3.953 | 2.610 |
| 9_12668288 | 7 | 138,224,010 | C | G | 96 | 4.634 | 2.964 |
| 9_12621880 | 7 | 138,940,786 | A | G | 97 | 4.634 | 2.964 |
| 9_12643258 | 7 | 139,074,924 | T | C | 98 | 4.634 | 2.964 |
| 9_12622014 | 7 | 139,301,604 | G | T | 99 | 4.414 | 2.892 |
| 9_12621999 | 7 | 141,821,119 | G | C | 100 | 3.759 | 2.515 |
| 9_12632690 | 7 | 141,821,210 | C | G | 101 | 3.987 | 2.618 |
| 9_67924733 | 7 | 141,821,227 | A | T | 102 | 4.259 | 2.787 |
| 9_12668141 | 7 | 147,734,794 | A | T | 103 | 4.547 | 2.932 |
| 9_12622372 | 7 | 148,065,669 | C | T | 104 | 3.204 | 2.168 |
| 9_12622381 | 7 | 148,065,789 | C | T | 105 | 3.204 | 2.168 |
| 9_12668586 | 7 | 148,159,012 | C | T | 106 | 4.215 | 2.762 |
| 9_12622286 | 7 | 148,272,987 | A | C | 107 | 4.069 | 2.680 |
| 9_12667535 | 7 | 148,349,039 | T | C | 108 | 5.429 | 3.273 |
| 9_12622322 | 7 | 148,462,294 | T | C | 109 | 3.601 | 2.404 |
| 9_12622469 | 7 | 148,696,027 | G | A | 110 | 3.042 | 2.070 |
| 9_12622428 | 7 | 149,060,593 | A | G | 111 | 4.659 | 2.964 |
| 9_12663161 | 7 | 149,061,350 | C | A | 112 | 4.659 | 2.964 |
| 9_12622453 | 7 | 149,250,370 | G | T | 113 | 3.153 | 2.141 |
| 9_12622411 | 7 | 149,250,395 | G | T | 114 | 3.153 | 2.141 |
| 9_12622311 | 7 | 149,691,895 | A | C | 115 | 4.478 | 2.927 |
| 9_12622524 | 7 | 149,697,659 | G | A | 116 | 4.736 | 2.970 |
| 9_12622644 | 7 | 150,908,721 | C | A | 117 | 3.977 | 2.618 |
| 9_12622850 | 7 | 159,173,360 | C | T | 118 | 3.581 | 2.395 |
| 9_12622938 | 7 | 159,573,860 | C | T | 119 | 3.924 | 2.604 |
| 9_12622889 | 7 | 159,584,567 | A | G | 120 | 3.924 | 2.604 |
| 9_12622788 | 7 | 159,604,492 | G | A | 121 | 3.402 | 2.286 |
| 9_12622857 | 7 | 163,439,022 | T | C | 122 | 3.295 | 2.228 |
| 9_12622948 | 7 | 163,439,866 | A | G | 123 | 3.140 | 2.131 |
| 9_12622910 | 7 | 163,716,317 | A | G | 124 | 3.152 | 2.141 |
| 9_12623261 | 7 | 167,425,189 | A | C | 125 | 3.590 | 2.398 |
| 9_12623241 | 7 | 167,630,881 | G | A | 126 | 3.981 | 2.618 |
| 9_12623281 | 7 | 167,720,497 | T | C | 127 | 4.072 | 2.680 |
| 9_12623101 | 7 | 173,092,102 | T | G | 128 | 4.515 | 2.932 |
| 9_12668149 | 7 | 173,270,813 | C | A | 129 | 3.177 | 2.151 |
| 9_12632876 | 7 | 178,249,562 | G | A | 130 | 3.758 | 2.514 |
| 9_12633515 | 7 | 180,307,479 | G | A | 131 | 3.514 | 2.359 |
| 9_12676615 | 9 | 974,242 | T | G | 132 | 4.084 | 2.687 |
| 9_12676601 | 9 | 1,005,035 | T | G | 133 | 3.414 | 2.293 |
| 9_12676434 | 9 | 1,021,644 | A | C | 134 | 3.582 | 2.395 |
| 9_12676612 | 9 | 1,024,365 | T | C | 135 | 3.479 | 2.340 |
| 9_12676456 | 9 | 1,445,358 | C | T | 136 | 6.393 | 3.773 |
| 9_12676911 | 9 | 3,447,627 | A | G | 137 | 3.221 | 2.176 |
| 9_12676821 | 9 | 7,353,784 | A | G | 138 | 3.389 | 2.279 |
| 9_12676927 | 9 | 8,013,654 | T | C | 139 | 6.751 | 3.909 |
| 9_12676796 | 9 | 8,302,477 | G | A | 140 | 3.163 | 2.145 |
| 9_12676868 | 9 | 9,407,326 | G | A | 141 | 3.925 | 2.604 |
| 9_12676984 | 9 | 10,507,728 | A | G | 142 | 3.833 | 2.547 |
| 9_12677123 | 9 | 10,516,440 | C | A | 143 | 3.630 | 2.426 |
| 9_12633240 | 9 | 12,697,877 | A | G | 144 | 3.049 | 2.074 |
| 9_12676990 | 9 | 13,109,730 | A | G | 145 | 3.909 | 2.594 |
| 9_12677005 | 9 | 13,110,823 | G | A | 146 | 4.141 | 2.723 |
| 9_12677144 | 9 | 13,140,908 | A | C | 147 | 4.648 | 2.964 |
| 9_12677210 | 9 | 13,153,883 | A | G | 148 | 3.912 | 2.596 |
| 9_12676980 | 9 | 13,449,360 | C | T | 149 | 3.235 | 2.186 |
| 9_12677172 | 9 | 13,707,254 | T | C | 150 | 3.506 | 2.356 |
| 9_12646023 | 9 | 20,784,904 | A | G | 151 | 3.514 | 2.359 |
| 9_12677402 | 9 | 21,633,820 | A | G | 152 | 3.732 | 2.498 |
| 9_12677449 | 9 | 21,677,503 | T | G | 153 | 3.824 | 2.542 |
| 9_12677440 | 9 | 21,679,884 | A | G | 154 | 3.893 | 2.583 |
| 9_12679045 | 9 | 83,793,513 | C | T | 155 | 3.021 | 2.056 |
| 9_12678966 | 9 | 84,190,900 | T | C | 156 | 3.505 | 2.356 |
| 9_12679160 | 9 | 84,190,925 | G | A | 157 | 3.474 | 2.337 |
| 9_12678970 | 9 | 84,225,762 | T | C | 158 | 3.505 | 2.356 |
| 9_12678958 | 9 | 84,822,522 | G | T | 159 | 3.381 | 2.273 |
| 9_12651486 | 9 | 90,618,689 | C | T | 160 | 3.552 | 2.378 |
| 9_12679559 | 9 | 95,105,653 | C | T | 161 | 3.373 | 2.272 |
| 9_12679501 | 9 | 95,421,027 | C | T | 162 | 3.048 | 2.074 |
| 9_12680150 | 9 | 101,308,862 | A | C | 163 | 3.108 | 2.113 |
| 9_12681065 | 9 | 110,058,275 | A | C | 164 | 3.258 | 2.202 |
| 9_12662909 | 9 | 111,068,507 | G | C | 165 | 4.562 | 2.934 |
| 9_12670820 | 9 | 111,795,368 | C | A | 166 | 4.108 | 2.701 |
| 9_12670816 | 9 | 111,795,374 | T | C | 167 | 4.220 | 2.765 |
| 9_12681007 | 9 | 113,098,874 | T | C | 168 | 3.874 | 2.572 |
| 9_12680969 | 9 | 113,419,960 | T | C | 169 | 4.039 | 2.657 |
| 9_23542737 | 9 | 113,853,656 | A | T | 170 | 4.504 | 2.931 |
| 9_12649193 | 9 | 114,152,389 | A | G | 171 | 4.344 | 2.857 |
| 9_12681243 | 9 | 116,434,271 | A | G | 172 | 5.300 | 3.231 |
| 9_12681270 | 9 | 117,144,362 | G | A | 173 | 5.794 | 3.475 |
| 9_12681214 | 9 | 117,502,354 | T | C | 174 | 4.595 | 2.951 |
| 9_12681555 | 9 | 120,108,301 | A | G | 175 | 5.116 | 3.152 |
| 9_12681443 | 9 | 121,998,408 | A | C | 176 | 3.545 | 2.376 |
| 9_12681580 | 9 | 122,117,106 | G | A | 177 | 4.234 | 2.772 |
| 9_12611838 | 9 | 124,178,905 | C | G | 178 | 3.950 | 2.609 |
| 9_12612119 | 9 | 124,182,398 | A | G | 179 | 4.601 | 2.952 |
| 9_12612126 | 9 | 124,182,786 | G | A | 180 | 5.404 | 3.273 |
| 9_12611816 | 9 | 124,183,138 | T | G | 181 | 5.236 | 3.218 |
| 9_12612124 | 9 | 124,183,878 | G | A | 182 | 4.502 | 2.931 |
| 9_12681809 | 9 | 125,270,943 | C | T | 183 | 4.529 | 2.932 |
| 9_12681731 | 9 | 125,271,875 | T | C | 184 | 3.879 | 2.574 |
| 9_12681716 | 9 | 125,823,977 | C | T | 185 | 4.771 | 2.978 |
| 9_12681699 | 9 | 125,910,054 | T | C | 186 | 5.024 | 3.138 |
| 9_12681742 | 9 | 126,074,752 | C | T | 187 | 4.457 | 2.914 |
| 9_12681852 | 9 | 126,077,381 | C | T | 188 | 4.701 | 2.964 |
| 9_12681817 | 9 | 126,211,114 | T | C | 189 | 5.024 | 3.138 |
| 9_12681734 | 9 | 126,212,234 | C | A | 190 | 4.701 | 2.964 |
| 9_12681798 | 9 | 126,277,420 | C | A | 191 | 4.562 | 2.934 |
| 9_12681845 | 9 | 126,312,700 | G | A | 192 | 3.981 | 2.618 |
| 9_12681661 | 9 | 127,013,134 | G | T | 193 | 3.745 | 2.509 |
| 9_12682116 | 9 | 130,777,524 | C | T | 194 | 3.045 | 2.072 |
| 9_12682058 | 9 | 130,885,701 | G | A | 195 | 5.632 | 3.367 |
| 9_12682099 | 9 | 131,693,936 | T | C | 196 | 3.525 | 2.365 |
| 9_12682055 | 9 | 132,374,915 | C | T | 197 | 3.980 | 2.618 |
| 9_12666554 | 9 | 132,744,132 | A | G | 198 | 3.866 | 2.568 |
| 9_12681964 | 9 | 133,460,917 | C | T | 199 | 3.140 | 2.131 |
| 9_12682222 | 9 | 134,252,870 | T | C | 200 | 4.103 | 2.698 |
| 9_12669047 | 9 | 135,224,204 | C | T | 201 | 3.885 | 2.579 |
| 9_12682286 | 9 | 135,944,370 | C | T | 202 | 3.711 | 2.483 |
| 9_12682265 | 9 | 136,370,529 | A | C | 203 | 3.100 | 2.108 |
| 9_12682230 | 9 | 136,755,180 | C | T | 204 | 3.554 | 2.378 |
| 9_12682123 | 9 | 136,758,864 | T | C | 205 | 3.412 | 2.292 |
| 9_12612193 | 9 | 137,493,484 | G | A | 206 | 3.354 | 2.260 |
| 9_12682516 | 9 | 139,526,358 | T | C | 207 | 3.337 | 2.251 |
| 9_12682596 | 9 | 140,002,725 | C | T | 208 | 4.733 | 2.970 |
| 9_12682398 | 9 | 140,011,231 | T | C | 209 | 4.603 | 2.952 |
| 9_12661722 | 9 | 140,318,492 | A | T | 210 | 4.436 | 2.904 |
| 9_12682582 | 9 | 140,618,740 | A | G | 211 | 3.545 | 2.376 |
| 9_12682396 | 9 | 140,705,251 | A | G | 212 | 3.545 | 2.376 |
| 9_12682386 | 9 | 141,484,086 | C | T | 213 | 3.586 | 2.397 |
| 9_12682412 | 9 | 141,572,995 | A | G | 214 | 3.018 | 2.056 |
| 9_12682544 | 9 | 141,813,796 | A | G | 215 | 3.586 | 2.397 |
| 9_12682704 | 9 | 142,109,686 | A | G | 216 | 3.348 | 2.258 |
| 9_12682809 | 9 | 142,589,380 | C | T | 217 | 3.448 | 2.319 |
| 9_12676667 | 9 | 148,911,079 | G | A | 218 | 3.168 | 2.147 |
| 9_12682908 | 9 | 148,994,565 | C | T | 219 | 3.916 | 2.599 |
| 9_12608342 | 9 | 156,054,746 | G | A | 220 | 3.185 | 2.159 |
| 9_12667183 | 9 | 156,767,924 | A | G | 221 | 3.194 | 2.163 |
| 9_12608743 | 9 | 157,816,936 | A | G | 222 | 3.398 | 2.286 |
| 9_12608555 | 9 | 158,560,296 | C | T | 223 | 3.304 | 2.230 |

Key for Table 4: Favorable Allele=allele significantly associated with MIR162 conversion success, Unfavorable Allele=allele not significantly associated with MIR162 conversion success, Position is mapped to the ZmB73 refence genome, version 5 (B73_ver5) available as of the filing date (see, e.g., maizegdb.org/genome/assembly/Zm-B73-REFERENCE-NAM-5.0 or ncbi.nlm.nih.gov/assembly/GCF_902167145.1 for full assembly). Locus.log P refers to the strength of the association for the locus with the MIR162 conversion trait measured as −log 10 transformed P (probability of the occurrence given the event). The P value was calculated with the Wald test based on the T-statistic. Locus.log FDR is a measure of false discovery rate (FDR), which is the expected ratio of the number of false positive results to the number of total positive test results.

### Example 3. Publicly Available Lines that Contain the Favorable Alleles

Publicly available maize lines were screened and it was determined, using methods described in Section IV of this disclosure, that the following lines comprised one or more of the favorable alleles at respective loci as shown in Table 3 and Table 4. The variety codes and the published patent literature describing these varieties are shown in Table 5. Each of the listed patents and patent applications is incorporated herein by reference in its entirety.

TABLE 5
Publicly available maize lines

| Variety | Patent literature |
|---|---|
| code | disclosing the variety |
| NP2174 | U.S. Pat. No. 6,844,489 |
| GAQ2025 | U.S. Pat. No. 10,004,195 |
| HAF2482 | USSN 17/522340 |
| IAX3489 | U.S. Pat. No. 9,504,217 |
| ADAA3608 | US20220151182 |
| GN05-61211 | U.S. Pat. No. 7,276,651 |
| G06-NP2760 | U.S. Pat. No. 7,371,943 |
| FF6224 | U.S. Pat. No. 9,332,700 |
| MFF7398 | USSN 16/774062 |
| AA2205 | U.S. Pat. No. 8,853,505 |
| GAB2010 | U.S. Pat. No. 9,756,811 |
| NPFA6899 | U.S. Pat. No. 8,742,233 |
| NP2460 | U.S. Pat. No. 7,122,726 |
| FF6788 | U.S. Pat. No. 8,946,527 |
| FX6134 | U.S. Pat. No. 9,992,966 |
| JCR503 | U.S. Pat. No. 6,635,808 |
| FX6278 | U.S. Pat. No. 10,681,886 |
| HID3483 | U.S. Pat. No. 9,035,145 |
| HID3753 | U.S. Pat. No. 9,332,715 |
| G06-NP2593 | U.S. Pat. No. 7,435,884 |
| G06-NP2623 | U.S. Pat. No. 7,323,626 |
| NP2171 | U.S. Pat. No. 6,759,579 |
| JRFX6624 | USSN 17/522202 |
| NPCI6621 | U.S. Pat. No. 8,258,379 |
| NP2222 | U.S. Pat. No. 6,710,233 |

### Example 4. Allelic Combination Data

The most highly correlated SNPs from each chromosomal loci in Table 4 were used to create allelic combinations that also correlated with the MIR162 conversion phenotype. Each data set had an N of at least 3. The allelic combinations are shown below in Table 6 and Table 7. The combinations in Table 6 could be used to positively select varieties that are likely to have a successful MIR162 conversion and the combinations in Table 7 could be used to eliminate varieties that are likely to have an unsuccessful MIR162 conversion.

TABLE 6
Allelic combinations correlated with successful MIR162 event conversion

| | Chromosome | | | | | | |
|---|---|---|---|---|---|---|---|
| | Chr 5 | Chr 2 | Chr 6 | Chr 9 | Chr 7 | | |
| | 129,259,011 | 243,529,534 | 158,572,433 | 8,013,654 | 134,444,680 | | |
| B73_ver5 bp | SEQ ID | SEQ ID | SEQ ID | SEQ ID | SEQ ID | Success | Failure |
| pos | NO: 2 | NO: 52 | NO: 88 | NO: 139 | NO: 94 | Rate | Rate |
| Combo 1 | C | A | A | T | T | 100% | 0% |
| Combo 2 | C | A | A | C | T | 100% | 0% |
| Combo 3 | C | A | G | C | T | 80% | 20% |
| Combo 4 | C | G | A | C | T | 93% | 7% |
| Combo 5 | C | G | G | C | T | 86% | 14% |
| Combo 6 | C | G | G | C | C | 86% | 14% |
| Combo 7 | A | A | A | T | T | 81% | 19% |
| Combo 8 | A | A | G | T | T | 81% | 19% |
| Combo 9 | A | A | A | C | T | 82% | 18% |
| Combo 10 | A | G | A | T | T | 83% | 17% |
| Combo 11 | A | G | A | C | T | 62% | 38% |
| Combo 12 | A | A | G | C | T | 59% | 41% |

TABLE 7
Allelic combinations correlated with failed MIR162 event conversion

| | Chromosome | | | | | | |
|---|---|---|---|---|---|---|---|
| | chr5 | chr2 | chr6 | chr9 | chr7 | | |
| | 129,259,011 | 243,529,534 | 158,572,433 | 8,013,654 | 134,444,680 | | |
| B73_ver5 bp | SEQ ID | SEQ ID | SEQ ID | SEQ ID | SEQ ID | Success | Failure |
| pos | NO: 2 | NO: 52 | NO: 88 | NO: 139 | NO: 94 | Rate | Rate |
| Combo 1 | A | G | G | T | T | 47.37% | 52.63% |
| Combo 2 | A | G | G | C | T | 8.11% | 91.89% |
| Combo 3 | A | A | G | C | C | 33.33% | 66.67% |
| Combo 4 | A | G | G | T | C | 33.33% | 66.67% |
| Combo 5 | A | G | G | C | C | 7.69% | 92.31% |

### Example 5. Breeding to Rescue the Conversion Failure Phenotype

A first parent maize plant containing a favorable allele from any of the SNPs in Table 4, such as at chromosome 2 at position 243529534, chromosome 5 at position 129259011, chromosome 6 at position 158572433, chromosome 7 at position 134444680, or chromosome 9 at position 8013654, or containing an allelic combination in Table 4, or the TE described in Example 6 is crossed to a second parent maize plant that does not contain such allele(s) or allelic combination and would otherwise fail MIR162 event conversion. The progeny are then backcrossed for at least two generations to the second parent maize plant to fix the favorable allele(s) or allelic combination or TE into the second parent maize plant's genetic background. It is expected that fixation of the favorable allele(s) or allelic combination or TE will improve the likelihood of a successful MIR162 event conversion.

Alternatively, genome editing (e.g., using a CRISPR enzyme such as Cas9 or Cpf1, or a variant thereof) is used to introduce the favorable allele from any of the loci in Table 4, such as at chromosome 2 at position 243529534, chromosome 5 at position 129259011, chromosome 6 at position 158572433, chromosome 7 at position 134444680, or chromosome 9 at position 8013654, or an allelic combination in Table 4, or introduce the TE into a maize plant that does not contain such allele(s) or allelic combination and would otherwise fail MIR162 event conversion. It is expected that introduction of the favorable allele(s) or allelic combination will improve the likelihood of a successful MIR162 event conversion.

### Example 6. Functional Annotation of Chr 5 Locus

The locus on chromosome 5 is devoid of any genes, but there are potential function regions based on low/no methylation. The region has no accessible chromatin regions (ACR) or histone modifications. The region is also devoid of any known miRNA. The region does have several predicted repetitive elements, notably a long terminal repeat (LTR) transposable element (TE). The sequence of the TE is SEQ ID NO: 232 and is located at Chromosome 5: 130436801-130439201 in B73_ver5. A blast search of the TE identified a region consisting of 2,340 nucleotides on chromosome 2 on B73_v5, and the region has 100% identity along its length to the TE on chromosome 5. This region on chromosome 2 is upstream of indole-3-acetic acid amido synthetase, aka., aas2, or auxin synthetase2). The maize allele of aas2 is Zm00001d006753, which was found to be highly expressed in maize embryo, anther, tassel, and spikelets. The region on chromosome 2 also contains ACRs from maize ear tissue, as well as H3K56Ac marks that suggest active transcription in ear primordia and young leaf tissue. The results related to the region on chromosome 2 are consistent with the hypothesis that the region mapped by GRAF identified a TE on chromosome 5 that carries a regulatory region. It is possible that this regulatory region may impact the area near the locus on chromosome 5. The nearest flanking gene is SWEET4c, known to be involved in sucrose transport to developing kernels. The presence of TE (SEQ ID NO: 232, present in certain genetic backgrounds like NP2222) or a synthetic TE (SEQ ID NO: 233, present in certain genetic backgrounds such as NPCI6621) on chromosome 5 is positively correlated with conversion success (see Table 8). One of ordinary skill in the art would understand that the sequence of the TE can vary to some degree due to the highly repetitive nature of such sequences.

TABLE 8
Association of TE with MIR162 conversion success

| Genetic | Chr5 TE | Conversion | Conversion |
|---|---|---|---|
| background | present | Success | Fail |
| Variety A | Yes (SEQ ID | 139 | 30 |
| | NO: 232) | | |
| Variety B | Yes (SEQ ID | 143 | 13 |
| | NO: 233) | | |
| Variety C | No | 72 | 98 |

### REFERENCES

1. Bernardo R., and Yu J. (2007). Prospects for genome wide selection for quantitative traits in maize. Crop Sci., 47, 1082-1090. DOI: 10.2135/Cropsci2006.11.0690
2. Breiman, L. (2001). Random forests. Mach. Learn., 45, 5-32. doi: 10.1023/A:1010933404324
3. Chen, L., et al. (2015). A cascade of sequentially expressed sucrose transporters in the seed coat and endosperm provides nutrition for the Arabidopsis embryo. Plant Cell, 27, 607-19
4. Chen, L., et al. (2012). Sucrose efflux mediated by SWEET proteins as a key step for phloem transport. Science, 335, 207-11
5. Guo, Z., et al. (2012). Evaluation of genome-wide selection efficiency in maize nested association mapping populations. Theor. Appl. Genet., 124, 261-275
6. Meuwissen, T. H., et al. (2001). Prediction of total genetic value using genome-wide dense marker maps. Genetics, 157, 1819-1829
7. Yu, J., et al. (2006). A unified mixed-model method for association mapping that accounts for multiple levels of relatedness. Nat. Genet., 38, 203-208

1. A method of producing a MIR162 plant with increased fertility, said method comprising:
selecting a first maize plant comprising one or more fertile QTLs, wherein the one or more fertile QTLs comprise one or more marker loci of SEQ ID NOs: 1-223 or 255, wherein at least one marker locus of the one or more marker loci comprises a favorable allele at position number 100 as shown in Table 3 or Table 4 or Table 9;
crossing the first maize plant with a second maize plant to produce a plurality of progeny plants, wherein the first maize plant and/or the second maize plant comprises maize event MIR162; and
selecting from the plurality of progeny plants a selected progeny MIR162 plant comprising one or more fertile QTLs.

2. A method of producing a MIR162 plant with increased fertility, said method comprising:
selecting a first maize plant comprising one or more marker loci linked to and within 10 centimorgans (cM) of one or more fertile QTL, wherein the one or more fertile QTLs are selected from the group consisting of
(i) a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3,
(ii) a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4,
(iii) a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4,
(iv) a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4,
(v) a QTL located on chromosome 8 at position 14,787,385 comprising the marker locus of SEQ ID NO: 255, wherein the marker locus comprises a favorable allele at position number 100 as shown in Table 9, and
(vi) a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4;
crossing the first maize plant with a second maize plant to produce a plurality of progeny plants, wherein the first maize plant and/or the second maize plant comprises maize MIR162 event; and
selecting from the plurality of progeny plants a selected progeny MIR162 plant comprising the one or more fertile QTL using the one or more marker loci linked to and within 10 centimorgans (cM) of the one or more fertile QTL.

3.-4. (canceled)

5. The method of claim 1, wherein the MIR162 event is introduced to the selected maize plant by transforming or breeding in a Vip3A coding sequence into the selected maize plant.

6. The method of claim 1, wherein the method further comprises producing an inbred MIR162 plant from the selected maize plant containing MIR162.

7. The method of claim 1, wherein the first maize plant comprises a QTL associated with increased fertility, and the QTL comprises SEQ ID NO: 2, with a C at the position number 100.

8. The method of claim 1, wherein the one or more fertile QTLs comprise one or more of:
SEQ ID NO: 1 with a G at position number 100,
SEQ ID NO: 2 with a C at position number 100,
SEQ ID NO: 3 with a G at position number 100,
SEQ ID NO: 4 with an A at position number 100, or
SEQ ID NO: 5 with a C at position number 100.

9. The method of claim 1, wherein the one or more fertile QTLs comprise SEQ ID NO: 2, 52, 88, 139, and 94, wherein position number 100 of each sequence, respectively and in combination, is the one of the following:
(i) C, A, A, T, and T;
(ii) C, A, A, C, and T;
(iii) C, A, G, C, and T;
(iv) C, G, A, C, and T;
(v) C, G, G, C, and T;
(vi) C, G, G, C, and C;
(vii) A, A, A, T, and T;
(viii) A, A, G, T, and T;
(ix) A, A, A, C, and T;
(x) A, G, A, T, and T;
(xi) A, G, A, C, and T; or
(xii) A, A, G, C, and T.

10. A method of producing a MIR162 plant with increased fertility, said method comprising:
selecting a first maize plant comprising a transposon element (TE) comprising SEQ ID NO: 232, 233, or a variant thereof that is at least 90% identical to SEQ ID NO: 232 or 233;
crossing the first maize plant with a second maize plant to produce a plurality of progeny plants, wherein the first maize plant and/or the second maize plant comprises maize MIR162-event-event; and
selecting from the plurality of progeny plants a selected progeny MIR162 plant comprising the transposon element.

11. The method of claim 10, wherein the transposon element is on chromosome 5 130436801-130439201 in B73_ver5.

12. The method of claim 1, wherein the one or more fertile QTLs or the TE is introduced to the first maize plant by genome editing or by breeding.

13. (canceled)

14. The method of claim 1, wherein the first maize plant and/or the second maize plant is an elite maize line.

15. The method of claim 1, wherein the increased fertility is increased male fertility.

16. The method of claim 1, wherein the method further comprises using the selected progeny MIR162 plant as a pollinator in a second cross with itself or a third maize plant.

17. The method of claim 16, wherein the third maize plant expresses a polynucleotide or polypeptide of interest.

18.-21. (canceled)

22. A breeding program comprising the method of claim 1.

23.-33. (canceled)

34. A kit for genotyping a plant wherein the kit comprises reagents for detecting the presence of
(a) one or more marker loci of SEQ ID Nos: 1-223 or 255;
(b) one or more marker loci linked to and within 10 centimorgans (cM) of one or more fertile QTL, wherein the one or more fertile QTLs are selected from the group consisting of
(i) a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3,
(ii) a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4,
(iii) a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4,
(iv) a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4,
(v) a QTL located on chromosome 8 at position 14,787,385 comprising the marker locus of SEQ ID NO: 255, wherein the marker locus comprises a favorable allele at position number 100 as shown in Table 9, and
(vi) a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4; and/or
(c) a transposon element (TE) of SEQ ID NO: 232, 233, or a variant thereof that is at least 90% identical to SEQ ID NO: 232 or 233.

35. (canceled)

36. A method of detecting one or more fertile QTLs in an MIR162 plant, the method comprising genotyping the plant at one or more marker loci linked to one or more fertile QTL, wherein the fertile QTL comprises one or more marker loci of SEQ ID Nos 1-223, and wherein at least one marker locus of the one or more marker loci comprises a favorable allele at position number 100 as shown in Table 3 or Table 4 or Table 9.

37. A method of detecting one or more marker loci linked to and within 10 centimorgans (cM) of one or more fertile QTL, the method comprising genotyping one or more fertile QTLs from the group consisting of
(i) a QTL located on chromosome 5 between 126407035 and 137510449 comprising one or more marker loci of SEQ ID NOs: 1-5, wherein at least one marker locus of the one or more marker loci comprises a favorable allele at position number 100 as shown in Table 3,
(ii) a QTL located on chromosome 2 between 220,204,265 and 244,411,717 comprising one or more marker loci of SEQ ID NO: 6-55, wherein at least one marker locus of the one or more marker loci comprises a favorable allele at position number 100 as shown in Table 4,
(iii) a QTL located on chromosome 6 between 146,526,712 and 168,005,927 comprising one or more marker loci of SEQ ID NO: 56-89, wherein at least one marker locus of the one or more marker loci comprises a favorable allele at position number 100 as shown in Table 4,
(iv) a QTL located on chromosome 7 between 130,409,997 and 180,307,479 comprising one or more marker loci of SEQ ID NO: 90-131, wherein at least one marker locus of the one or more marker loci comprises a favorable allele at position number 100 as shown in Table 4,
(v) a QTL located on chromosome 8 at position 14,787,385 comprising the marker locus of SEQ ID NO: 255, wherein the marker locus comprises a favorable allele at position number 100 as shown in Table 9, and
(vi) a QTL located on chromosome 9 between 974,242 and 158,560,296 comprising one or more marker loci of SEQ ID NO: 132-223, wherein at least one marker locus of the one or more marker loci comprises a favorable allele at position number 100 as shown in Table 4.

38. The method of claim 36, wherein the one or more fertile QTLs comprise one or more of:
SEQ ID NO: 1 with a G at position number 100,
SEQ ID NO: 2 with a C at position number 100,
SEQ ID NO: 3 with a G at position number 100,
SEQ ID NO: 4 with an A at position number 100, or
SEQ ID NO: 5 with a C at position number 100.

39. The method of claim 36, wherein the one or more fertile QTLs comprise SEQ ID NO: 2, 52, 88, 139, and 94, and wherein position number 100 of each sequence, respectively and in combination, is the one of the following:
(i) C, A, A, T, and T;
(ii) C, A, A, C, and T;
(iii) C, A, G, C, and T;
(iv) C, G, A, C, and T;
(v) C, G, G, C, and T;
(vi) C, G, G, C, and C;
(vii) A, A, A, T, and T;
(viii) A, A, G, T, and T;
(ix) A, A, A, C, and T;
(x) A, G, A, T, and T;
(xi) A, G, A, C, and T; or
(xii) A, A, G, C, and T.

40. The method of claim 36, wherein the genotyping is performed by sequencing, polymerase chain reaction (PCR), probe hybridization, or single base extension.


SEQUENCE LISTING (255 sequences)

SEQ ID NO: 1 - DNA, 200 bp, synthetic construct
  source 1..200: mol_type = other DNA
ttcatatttggctagtgttgattatacctactttgtgatacgagtcatcttagcatgttgcagcggcttcgcttactcagacagggtatcctatctttanaggaattcaacagacttgattagtgagtgccttttggatggttcgcgaatcagtcatcgtataatgacccttctaaggattgtttgtctctcgtagagtt

SEQ ID NO: 2 - DNA, 200 bp, synthetic construct
  source 1..200: mol_type = other DNA
gtgtgtggcatgattaaaagtgtttgcttaaattaaaaaaagaaaaatatataaatgggaaactgctcacatgggccacgttttcccacaactaggccanccaagccaacgacccagctagcccattcccacgctagcccaactccactgcatcttgtgaccagtaatccatttagcgaccggtcaccaccatgcagggt

SEQ ID NO: 3 - DNA, 200 bp, synthetic construct
  source 1..200: mol_type = other DNA
ccgtgtcctaggcatatcgtcgtgctacatcattggctagctgatggcaacgtgcaacgattatttgcatgtcgcgatcgcacagcacaactcgacactntcaatcagccgttgaagccagccgaacaagactggccgtccatgcatgtccgttccagccgagcgacacgagtgtcccgatcgagtcactcactcatctt

SEQ ID NO: 4 - DNA, 200 bp, synthetic construct
  source 1..200: mol_type = other DNA
gatcacgcgcgcgtggcgtggcgcccctcgtcatcagcacagtacacagataggatcgagctagtagcaactacttactgttcattgatgagatatgcgnatgtgtatttattaatttgctgtctgtctggcactgtcactgtgtggcctagtactacatcgatctactcaccgatcttggccagctcctcgaagaactt

SEQ ID NO: 5 - DNA, 200 bp, synthetic construct
  source 1..200: mol_type = other DNA
tttttatttattagttagcaggatatttatctgctttctgaagttatttctgaaatgagcttaatccatatttgtttgtgctatttcccaaccaatacantctagtttctttgcttgagctgcatgtgaccactgcactacttgactaatgtctattaggataattctggagcagtccctctcgtagcaaagcaccataa

SEQ ID NO: 6 - DNA, 200 bp, synthetic construct
  source 1..200: mol_type = other DNA
acgatcgagttgcgatgcgctggggtgcggttggatagatggtagcagggatagtggaggcgaactttatgtatacgacccgcgacggtacttggccttncggctcggctcgtcgtcctgtcctggcagcgcgcaggaaggtgcccgtctgcttccgccaagtacttggccttgcggctgcgagagggatagctgacggc

SEQ ID NO: 7 - DNA, 200 bp, synthetic construct
  source 1..200: mol_type = other DNA
gccacaccatgccacgctatatggatataataatgccagtgctaagagattctgtttgcgtttagtgttactgttaccactgactaatactaatgcatgnatttgcatactaaggctttagtgagcacagaagattctgatattattttatccatatagggttcttaatgcagcagcacaaaggcaagctagagaagctg

SEQ ID NO: 8 - DNA, 200 bp, synthetic construct
  source 1..200: mol_type = other DNA
tttttctagaagtctaaaaataatttgtatccaaacagggtctaaaagggttaaaaaactggataagcattgttttgcatcatacttcaccaagaagccngaggaaatagtcgaactgcagatatgtgaaattcgttcataatttgtccttgctatcactgttgtcacccctgaaagtttctgtttggactttaaaagtg

SEQ ID NO: 9 - DNA, 200 bp, synthetic construct
  source 1..200: mol_type = other DNA
ttctttctatgttgagcactcgatgcgagagactgtcaaggctagacaggctgccgttgttgagcttgagcgatgtagattacaggatcgtgaaaggccngctctatagatagactggcaattggcaccaaccactacacgcacttgtttctacacgaaggaccatgccgcagcatggagcgcatgacgcccagtcagat

SEQ ID NO: 10 - DNA, 200 bp, synthetic construct
  source 1..200: mol_type = other DNA
ggatcaaatggatgcatgggggtacatgctgttgcacagcgctctcttctcttgtgtgctgcgtgctgcgtgctgcttccaaacgctagctacgcttaanccgactcgcatgtttcgcagtcggggagcgccttgcccgcgaggaagccctggacaagcacgagcgctcgctttggctggtagctgggcacctcgtgccc

SEQ ID NO: 11 - DNA, 200 bp, synthetic construct
  source 1..200: mol_type = other DNA
agggaattggcccaaaaacgaaccgaaaccatctagcagtaaagcaagcgagtcttgacagatctggccaaagcacagccggataccgcgtatagagtgnagagaaacaacagatcagaccatgtccaattccaatcacaccgccacagagcagcgcacagctgagccgaaaccaacaaccgcttccggtggccaggaat

SEQ ID NO: 12 - DNA, 200 bp, synthetic construct
  source 1..200: mol_type = other DNA
agaaaaaggtctgattatgtacaaatctgcgatggacgggacgcatccgatgaagaacgggatgaatcgcaagaggaagaggacaattccttaccgaggnaatttccggtgtccacgcacccccaacgaagagccgaagaggctgctgggcctggaccctggagtgccgctgcgaactcggctggtggttgaacagagtg

SEQ ID NO: 13 - DNA, 200 bp, synthetic construct
  source 1..200: mol_type = other DNA
tcaacaagccctttgtagacatcttgtggcctctcatgaaaagttcttaggtcctgatttctgcatatagaacatgaagaagaaatatattactcgctgncagtggcaaatgtatttatctaagctcttaaaaatcctttttgtatggcatgccaatttgttatattttgagaagcgttttctgcgttacctgaagctga

SEQ ID NO: 14 - DNA, 200 bp, synthetic construct
  source 1..200: mol_type = other DNA
ttgcaaaatttagagactggtcaagcactacaccaagtactaaccatgtgttggcatggtacagcttctgaaaaaaacaatgcatcagctacctaacatnaagctacgatgcaacagtgggatacccctcccacatgaatttattattgaaaaaacatataaatgctcaccatcccgcagttgagcctgctgctccatgg

SEQ ID NO: 15 - DNA, 200 bp, synthetic construct
  source 1..200: mol_type = other DNA
cttctgaaaaaaacaatgcatcagctacctaacatgaagctacgatgcaacagtgggatacccctcccacatgaatttattattgaaaaaacatataaangctcaccatcccgcagttgagcctgctgctccatggcctgcaaccttatcttcagctcagcattttctgccgaaagtccagttgtgtctctctgccgagt

SEQ ID NO: 16 - DNA, 200 bp, synthetic construct
  source 1..200: mol_type = other DNA
cattttctgccgaaagtccagttgtgtctctctgccgagtaaaacaaaacagtgtgaagaatattaagcacagtaatacaagagtaaattgcctggaccntaaagtattacgtgattctcgtttggatcagtctatttggctccctaacatattttactaatcttatcccggtcttaattagacatggggatgcctagaa

SEQ ID NO: 17 - DNA, 200 bp, synthetic construct
  source 1..200: mol_type = other DNA
cgaggctggctctgggcaccgatgacgccgccagcggcgactgctccagcacccggaatgggtcgtcgaggagcatcaggtcccacagcccgccaccagngcgcccgtacggtaccagcgcggcgaccggagccgcaaggcacgccattgtcaccaccgctaccgcgaggacgacgacggccgctgaagccgacgccttc

SEQ ID NO: 18 - DNA, 200 bp, synthetic construct
  source 1..200: mol_type = other DNA
ctccaagaagagaaggatggaggccctctgttaaaaatgaagttattgcactgcttctagattggccgtgcagagttattgagcatttccatgaagaaangatatggaaaaacttatttggtttctattattgtttggaagaaatccgctgtaaggttctactgaatgccaagaaactgccaacagtttgctagcttcaa

SEQ ID NO: 19 - DNA, 200 bp, synthetic construct
  source 1..200: mol_type = other DNA
tagagcggtatgcgtttttatatgatgtttcaatatcgcatttgttttgtaaatgcattatcttatattgtttcatgaattacagattgtggaacttaangatgagaggaactggagaagtgggcttagagttcgattgttgagcacttgcatggtaaccttccattgaacttcattaattgccgatgagatggcttatt

SEQ ID NO: 20 - DNA, 200 bp, synthetic construct
  source 1..200: mol_type = other DNA
cacgcacctccacctggcactgcagcgactgtacgtagtttatgatctcgtccagcatcacggccttcccggtaaccttcacgaggtcgaggagtctttngaaattgaaatatgcgtgtgttttcaaccgaattttgagcaacggccgtcgatgtttacttaccttgctgcagcctggcacgaggtcctgcagcagcttc

SEQ ID NO: 21 - DNA, 200 bp, synthetic construct
  source 1..200: mol_type = other DNA
aagtaaaaatggatcttccaaggatactgacaaaacaagtaaaggctctaaaagagtccatgaagaatcagatagctctgaattaaatgatagaaagagngatatggataatagcgacgtggattcaaggccaagaaagaaaagggcagaaaaggctaaagtcaccaaaaaacagaaaatagttacaaatgagaggaaac

SEQ ID NO: 22 - DNA, 200 bp, synthetic construct
  source 1..200: mol_type = other DNA
tgtgttttcttgttgactctggtaaagattttttttcgaaggacacccaaaacgggcagggagctcctgcaattcacttaagaagaaagaattggcccantttataaggtaaattgggcttaaaacccatacaacttcatggttaattaagaaaaattggcaaaaacttaaaaggatacatcgggtgcaaccatatctat

SEQ ID NO: 23 - DNA, 200 bp, synthetic construct
  source 1..200: mol_type = other DNA
acaagattagcaagagtagtcttgcctaaccctccaaagccagcaatagacagtacaactggctgcttggatgcctccttgtcgccctccatgagccttntcacaaggtcactgctcttctcttcggcgccaacaagttctgtcgcctttctgtaaagagccgacagccgcaggttgtcgatgcttctgccaacaggctt

SEQ ID NO: 24 - DNA, 200 bp, synthetic construct
  source 1..200: mol_type = other DNA
cgaatgcaggtactgcaacgcttccgcggtcttgatcgcgatcctcatgcgtagatccagcgggaaatcctcctggctacacaggatgtcgaggatgctncctttagcgacgtactcgtagaggaaagcggtggtctggggttccagacagcagcccagaactctgacgatgttcctgtgggcgatcttggacacgacca

SEQ ID NO: 25 - DNA, 200 bp, synthetic construct
  source 1..200: mol_type = other DNA
gcttcctttagcgacgtactcgtagaggaaagcggtggtctggggttccagacagcagcccagaactctgacgatgttcctgtgggcgatcttggacacnaccatccctccgttgatgaacgcctcctcagagccctcgtagaccaatctatgcaccgccaccatcgtgttgtcctcaagcgttcctttataaacctcgg

SEQ ID NO: 26 - DNA, 200 bp, synthetic construct
  source 1..200: mol_type = other DNA
ggcagacacatgaagcttcaggggaagaggccggaacagggaagaaaaacagcttgtcagcttacttggcttgatgacgacgcatcagcgggcagcatcngtagcacgaggagctgaagcagcactagtagtgcagaagatgacacagagacaacgtgtggtctccagcccatctttggatttatgttctctctctgcta

SEQ ID NO: 27 - DNA, 200 bp, synthetic construct
  source 1..200: mol_type = other DNA
cttcatcaaagcatctatttttaaaaagtccacgaagaactatgttgtatgtacatatgtccatagcgataccactttctgtcatttcatggaatttcanctttgcaggaactgttctcccggcctgaaataacccatcaattatgatgctgtataaaatagttgaaggctttattcccctttgcagcatttctctgaaa

SEQ ID NO: 28 - DNA, 200 bp, synthetic construct
  source 1..200: mol_type = other DNA
tccacttgagtggcttattgccctttcgatctcttttatcttcaatttcagctctcgaactaaatgtacactaccttggggagaagcgaaatgcaagtangccttggccaaaattagctgatcttgcatcatccagatcttctctttggtgtctgggggagatttagatatcctttcatcttcttccgccgccgtttcgt

SEQ ID NO: 29 - DNA, 200 bp, synthetic construct
  source 1..200: mol_type = other DNA
ggaattaccaaggagcagaaatgccttcatccttagaccaagcttccggaacatgaatctctcttcatctgttattgtttcaggaggtctggaatcttcngttggcctcagggctgtctcaacctttcctagtaccctttcggctttctccatctttttctgtgcctgtacataaataaagtgaaattagttcaattcaa

SEQ ID NO: 30 - DNA, 200 bp, synthetic construct
  source 1..200: mol_type = other DNA
accaaggctcatgaggctgacatgcacgttctcagtttctcctctatatcaaaaactctagtcatttaaaatatcactacatatttcaatccacaaccanaaattcaaccgctactcacgcagcagcagcctgcgctccgggtgaaacatttctgtggaacgcgctcaggcttgtgcgtgcaatcggctccccgaacaac

SEQ ID NO: 31 - DNA, 200 bp, synthetic construct
  source 1..200: mol_type = other DNA
acggacaatagaagacactgggcgagacctttccctacctctagtgaagggaactatgcagaatgagcacatattattgcaacctctcatgattgacacnaatgccgtaattgaattgtcagagatccttactggggtgatatcagcataagtttcctcgagtgatagaagtgtgttcatgcccttgtggccgtaatcga

SEQ ID NO: 32 - DNA, 200 bp, synthetic construct
  source 1..200: mol_type = other DNA
tattgttattgttatttcattttacgttatgtccttagaagtactctactcgtggctaacattacaattttatttaaatacatccggttcttcccatatntgatacacatctattcgaactcatccggttcttcccatatctgatacacatttgaattggtgtcgtaactctcaaacgctcaattgttccatagacgtcg

SEQ ID NO: 33 - DNA, 200 bp, synthetic construct
  source 1..200: mol_type = other DNA
agctcctcagcgtaagcatcttcttctcagccattggtgttgactgaatgcttgcgactggacgacaggtcgaaatcctcgctaagtacgtgcggcgcgngacaggatgattgaatacgaacggaaggacgtgacctggagcagtcagcgtttggattgacgatgtttgaaaacgaacgaacggaagtccaggaatctgt

SEQ ID NO: 34 - DNA, 200 bp, synthetic construct
  source 1..200: mol_type = other DNA
gattgtagctttagcacgagctatatactagtatgaataaagaatcttttagccataaattttgtgggggttaaaactctagcaacgagctacatttccngtcttattttgtgctagtatttgcacttctaagcccagatagtccatatttcagtgttcaatttaattttgtcatatgtgtgaactagttcattaggact

SEQ ID NO: 35 - DNA, 200 bp, synthetic construct
  source 1..200: mol_type = other DNA
cacagttctttactaatccaatcaagccttcatctgtggtgccttcaacaaatcgcaagttcaatttcctaagcagcttgcatccttccccaatagcaanaagtcctggatctccaataaagcaagcctgcattttaaggtttgtcttcatactttgtccataaatatatattgggtctacaaaagcggaactgtatgga

SEQ ID NO: 36 - DNA, 200 bp, synthetic construct
  source 1..200: mol_type = other DNA
agcgtggccaagctcctcttccaaatcagcagctaccttctactcatcaacatcctggagcccttagtagccagccaaatatacctggagtcggtgggcnattttatcctgccaggaatcttcctgatggaccatcctctgctgagactaataacatgggttttatgaactcacctgctcaaatgcatcaaggtggagca

SEQ ID NO: 37 - DNA, 200 bp, synthetic construct
  source 1..200: mol_type = other DNA
atttcctatacctactttttatttacaatcaatttttcatatgctaacaattactcgtacgtaaacctactgttacaggaataaacagtgctgcagggtntgaacattaacacgctatatctagagggagagagaagagaaccgacgtgtagagccctgcggggtgcagcgctgcgggcgtagggtgcccccctgcaggc

SEQ ID NO: 38 - DNA, 200 bp, synthetic construct
  source 1..200: mol_type = other DNA
taacctcgtcatcaccatcaaacaggcagcgtctcaggagcacaaatatgcgaggctggataaaaagattgcaagtgcaatggacatttgacagatcagnaacattgcagagcaaggacatcattgaagtaaaaaaaaaacatgcaagtaccttgagtgagtcaaccaaggcaccaaactttgccaatgtgctgactgca

SEQ ID NO: 39 - DNA, 200 bp, synthetic construct
  source 1..200: mol_type = other DNA
ggccaccatcatatcggtcatcatcatagcgagcaaaacgatctgatcctccaacgcggtcaccacgtcccccagcgccaccaccataccttgaagcggnagaatatcttccagttctaccactgccatctgaataagggcattcacgagcccaatgtccagaacggccgcatttgaaacattcatcactcactgctgga

SEQ ID NO: 40 - DNA, 200 bp, synthetic construct
  source 1..200: mol_type = other DNA
ttgggaaaatttagcagtaacagttgcccctcacttttgatgtcatcgtgttgaaactgtggggaagaattcaacgaggtctggacgtctggtaggtagnaagtcatgttgaaaccggatagaattcaagggctgtagtcctgttgaccaactatgtctctgtgcctttctcctggatgtaactggtttgttctgtagat

SEQ ID NO: 41 - DNA, 200 bp, synthetic construct
  source 1..200: mol_type = other DNA
ccgactcggtcaccccatacaaaacttgcagcaaatttacagacacggacacagcgcacggtctctggcaggcagccaggttcttacaagggcaacgccnaaaaaaaaacaatccagcaccaggcacgctgtctgttgccggggcatgaggccatctttccgggtgatgtgagggtatatggattaagatgaaaaacggc

SEQ ID NO: 42 - DNA, 200 bp, synthetic construct
  source 1..200: mol_type = other DNA
tgtcccgctttcttcatggaagccctcgggtcttccctggatttgggggcgccctggacttagcagctggtccagtctttggagagcattgtgccttgcngccgatgtctttggagttcccgatgctgtggcggtgcagccggcagcacactgcgccgccgccgccgtttcctccctctggtgtgtgaggcgtgtcgctg

SEQ ID NO: 43 - DNA, 200 bp, synthetic construct
  source 1..200: mol_type = other DNA
tgtgtaggctgtcttcagcagatcgtgcatatggtcgcgtaaaatattgttttgtaccgtagactgcactattcacaggtgggggtgaagtttgaaatanaagatgggatggaagataggatgggtaagctgctggagacagccttacaagaggcatcaaatatgcgtgtacgaatgacacggtacaagcataattaccg

SEQ ID NO: 44 - DNA, 200 bp, synthetic construct
  source 1..200: mol_type = other DNA
gtgtctgcagaataaacccaccgcctccccatttagggatatggatcttgctaaaaggatatgattactcacttttccattgtctgcaagttctgcccantcataagcagcacttatctgcgcagctgcatcagtgacctccttccgcttttttgaagcattaccacgggaagctttccctgctgccgcttctgcctcct

SEQ ID NO: 45 - DNA, 200 bp, synthetic construct
  source 1..200: mol_type = other DNA
agcttaagagtatcattgccaataacgacttcatagctatttacctgggttagcaaattggcatgcagagaaatttatcagatcaatacgtttgaatatnttcgaagctttcttgatttgttcatctgttgcacgcggtgcatcatcagaagtcacatgaacctgcaaaccaagcaagaaaattagcttacagagatcgt

SEQ ID NO: 46 - DNA, 200 bp, synthetic construct
  source 1..200: mol_type = other DNA
ctccatctcctataagccggtcaagcatggcaggcccggtatcggtgccacccactcctcgcggtttattcctctgaaatgagctgttacccggctctgnagtctcccaagagtcagttggcaatgttcgttcagcttttctacttgttttgaacaagttaaatgcccaagatattttgttttgttccattccagctcaa

SEQ ID NO: 47 - DNA, 200 bp, synthetic construct
  source 1..200: mol_type = other DNA
aattgttaaaaacgaaggaaaccaagaatgttgcatctaaatccaaatgaatttgcttaagtaaactagtaccgctaattcagctactcagaatgtcgcnccacccaaatctaaatcagctaaaactgtggcagccaaagtatgtggcgcaggaaccacagatccccaatcggtctcaagccctagctagtcacacaagt

SEQ ID NO: 48 - DNA, 200 bp, synthetic construct
  source 1..200: mol_type = other DNA
atgttaccctgaaatgaaaaaccaacagacccctgtcaactgttagccatactcttgtggcatattgccacttgagctacaccacagaaagaatgaatangaacaatacagcaaaccaaaaatttatctacaaaagaaaatattggtagttgtacattagagccagccaaaatataaaagataaggtttgttgaagtcac

SEQ ID NO: 49 - DNA, 200 bp, synthetic construct
  source 1..200: mol_type = other DNA
tctgtggcttatcacatagtgcagcacagtgccattacaccatcatagaaacgctgtagggcaaccactacttattggactgttacatgttgatgcttcntacatattggactgttagcactacactgcaatgaaatgattgatgcttcttatttacatgcttaattatttgattgaatgagtcattgatatctatgtat

SEQ ID NO: 50 - DNA, 200 bp, synthetic construct
  source 1..200: mol_type = other DNA
aatggcgttccagtcgccggggtctgacaaggacgcgccattcccattcccattcccattatatgtaggattgtaggctagcaaccacacagacacaggnagatagatgggcacaaaatggattcatctcctccgccgccgccgttgagtcagctgcatgcccaagtgtcgcactagcaaagaacggatggcggcggcgg

SEQ ID NO: 51 - DNA, 200 bp, synthetic construct
  source 1..200: mol_type = other DNA
gattggttaaaccatgctctttctaggtaaatctatgtggccagcgtgtgctagacagatcactgagactcgtcataaccatggatcgaaagctacagtntcctagaccctggttcaggctcgccggagctcggctctctgcctccgccacggtcggagatgctctaggttatttgggatcgattaacctaggggacgta

SEQ ID NO: 52 - DNA, 200 bp, synthetic construct
  source 1..200: mol_type = other DNA
cctgtgcggggtctggggaagggtatctttaagcgtcaagccttaccagcataatatgcagaggctgaggctcgaacctgggaccttctggttacagtcngtaagctctaccgctgcaccaggccacaacaaagccttaatcaactgatgcttctaaaaccgcatctagaactagggatggtaatgggtcacgatccaaa

SEQ ID NO: 53 - DNA, 200 bp, synthetic construct
  source 1..200: mol_type = other DNA
agccggcagcagcaggtgcagtgggaccagcagacacatgcccaggcccagacaagctcatcatcgtcctccttcatgatgaggcaggatcagcagggantgccgcctccacacaacatctggtacacttttatgcccacttctacctagttctttgcctcaattatatatggtcaacgccattatcatgatacatcctg

SEQ ID NO: 54 - DNA, 200 bp, synthetic construct
  source 1..200: mol_type = other DNA
agggcgtccaactattgcaaatcatcaaacaaaaatgacagcttaagctgagctataaaaacactacaaaagcatcaatactgcaaacatctcaaccctnacgagaaggcgctgactttttttatataataaaaagataactaaggactgtctagagtctatttccagatattatcgtgcacagtacaaaccaatttcag

SEQ ID NO: 55 - DNA, 200 bp, synthetic construct
  source 1..200: mol_type = other DNA
tctttgaacaacgttttcaactaataatactacatgagtttattaacaacagaacaaggccacacctaacatgatgagcagcagcaacaccctaagcatnagtccagcagcgtcagatgcttactattagtggaatcaagatatgaagatagtgtgtagtattttagtagggtaggtatgatgaaacctgatgatccgaa

SEQ ID NO: 56 - DNA, 200 bp, synthetic construct
  source 1..200: mol_type = other DNA
atccatgtctcacatattaggaattgaatggagcggtatggccctactgggttactgcttcaagtccgcaagtcatctggaattctggataagttgttgnttcgtaccactctttaaaacatatatgcacgcatttaagatgctataatagagcgagccaattaatataaatgtgtttaccttgcctccggtagatttaa

SEQ ID NO: 57 - DNA, 200 bp, synthetic construct
  source 1..200: mol_type = other DNA
ccagtaaaaactattttagtaataccatgagtcataagtcacaagtgacatcccaacattatagaaatttaaataaagaaagaagaaagaaagataagtntgcacccattagttgtgcatgtttcaagaaaccctgactttctcccagtgtctgcagcatagtccatagtaggaagttctttcttataaagcattagcac

SEQ ID NO: 58 - DNA, 200 bp, synthetic construct
  source 1..200: mol_type = other DNA
caattttccctttttctaaatgttgtcaaatcaggcagacataacgaatgtgattctgataaggtacaacctttttcttaattcatgataccggaatggngaaacaaacgttacaaacttgattacctcaagcaaggacttggtttggctatctgcatcgcttggattgagaaggatgaactgagagccctcacctacac

SEQ ID NO: 59 - DNA, 200 bp, synthetic construct
  source 1..200: mol_type = other DNA
ctttcaactcccatccgcttgccgttgccgcgtttgtttatcgagccaatttgatcagggcgccacaccgaaccctagttcttgatttaatcacgggttntgtggtcatgccggccgggtcaccccgttcttcttctcttccacgagttcaggaggtagagtgagagagaacatggaacgacaggggatggagggcaaca

SEQ ID NO: 60 - DNA, 200 bp, synthetic construct
  source 1..200: mol_type = other DNA
gccgacctcacccccctcatggacggtggccgagccgcagccgtcggcggcgacccctctcccatcgctgctccttccaatcccccggcgctgagcagcnggcccgtcgatgtctcctcgttcacccataccccgtgctactggtaaccaacccctttgcccattttatcgatccatagtttctcgcagtagattcaagc

SEQ ID NO: 61 - DNA, 200 bp, synthetic construct
  source 1..200: mol_type = other DNA
ctgatctttatgttgttttcatatccaacgaggaaaaaaaggtgatttattgcttatttttctattgttggagatcttattttccttcagttggaaattncccttccattcaggttcctctctggcatcagaaagcaggccattctgatgatggatttatttgctgggattatcacgtgatatgcatccaggtaaagcta

SEQ ID NO: 62 - DNA, 200 bp, synthetic construct
  source 1..200: mol_type = other DNA
ttgcattggacatcacccgagaaccaagattcaatcaatagctgggggagttgttgtcattgctggcattcgaaatagcagatcacatcgcttgggtttnaattcaaggatgtatcagccaatcatcaagaccagggaacatttctggtacaatcccagttaccatgatgatacatggggaatgagatttgtatctcgtg

SEQ ID NO: 63 - DNA, 200 bp, synthetic construct
  source 1..200: mol_type = other DNA
tcgcttgggttttaattcaaggatgtatcagccaatcatcaagaccagggaacatttctggtacaatcccagttaccatgatgatacatggggaatgagntttgtatctcgtgggctccaggtaagctgccaggtgatagttggttaagccaacctcaaaaacattgtatggatttaaaactcccctcactcttcaaatg

SEQ ID NO: 64 - DNA, 200 bp, synthetic construct
  source 1..200: mol_type = other DNA
ccaacaatttatctgtcttgcctaatatttccttggaactttcttgaatcccgcttgcattaacttggattttctctactttgtcttgggctttccctgnctcctcttgctcttcccctgctttgtgtttgatcttcacatcatagcgagaaatccttccagacttcggcagtgcctctgatccaacaactcttaccgtc

SEQ ID NO: 65 - DNA, 200 bp, synthetic construct
  source 1..200: mol_type = other DNA
cgtaggagattatataaagaacagtacgtttccgcactgagctggtcaaaaccgttgcgaattctacagctcgctcatcagacaatatagtagtactctntctgtgggtagttttcatattacgatcacacggagaccaaaacaccctgctgaacggggtgtttggccgccgatctagtcaaccttttacgttagcaatg

SEQ ID NO: 66 - DNA, 200 bp, synthetic construct
  source 1..200: mol_type = other DNA
gtaggctggttatgcacatcatatcgcaaattcaaatgtgagcacatcatttgaatctccaagtgtgttctttgctaatcaattatttttctaaccctanggttggtggagcacatattttcttgattagaaaccttggagtggacgatcaaggacttaaaaaggccaaagagtcatagaggatcagctagaacatctac

SEQ ID NO: 67 - DNA, 200 bp, synthetic construct
  source 1..200: mol_type = other DNA
tgtctctttgcattctaagagaagatatctaggagaaacataacctgcccggcagtcttgttcaaagcccagttgaatgctggctgatcatttgacttcntttgcttcgaccagggctgctccttaagctcctcgatccacttccttaacaggagcttggcaccttcagtgggcctgaggaatagcatgcagctgcaaat

SEQ ID NO: 68 - DNA, 200 bp, synthetic construct
  source 1..200: mol_type = other DNA
aactggcaagtactcgtttgcttcatgggagactcttaggacagattttgatatcattctttaaacaatgccttgaggaaagcgtgatttatcgaaattnttcccgcaagcaagagagtgaatttgttttgagggcttaaggttgtgttgtgcgtgttggattggattgattaaaattcgtagtttcgtacagttttgtc

SEQ ID NO: 69 - DNA, 200 bp, synthetic construct
  source 1..200: mol_type = other DNA
ttttgtcaactacccttgaagatcttcttgcccttgctctttgctcagctggcgggtaattcagtaattgataactgtgcatcgttctttagcaaatatnttaacaatgaaatgggacggttgatgtgtaaccattttgatttgtccttcagtttttttgttttatcaagtttccctggtcggcggaagcatgcaataga

SEQ ID NO: 70 - DNA, 200 bp, synthetic construct
  source 1..200: mol_type = other DNA
aaataaatgaaattacaaaaacaccagaaaacacaaaactatgccttttctactctgtcctcatggccacaaggataagcttgcacttcaactacacatnacacctttaaaccaactccatcagaagtgggtctcctatggacattagccgaatcacctttatgaaatcaatctcattcataactttcacagggaaaaca

SEQ ID NO: 71 - DNA, 200 bp, synthetic construct
  source 1..200: mol_type = other DNA
ctaatttactatgaagtataaatcatggttgattgaaagttgcaaagaagagaagggttattggaatcagtaaacactcacttataagcgtgtgacaacntgcagcgttcaacagcaagctctgcattgctctgctcagcaagaatgataaccgtcttgaaaatccttccataaagcagcctccactcaagtgctgtgcg

SEQ ID NO: 72 - DNA, 200 bp, synthetic construct
  source 1..200: mol_type = other DNA
gcattcaccaatcatcttccacagctccccaggaagtcctacacctactactcttgaatattgaggcggtggcttcttttcttttacaacagacttacanatttcctctgcactcagtccagcccatctgcatttcaatgagcatataacaacaccattcacaaaacattaaggacaacaaattgaatatcatgctactt

SEQ ID NO: 73 - DNA, 200 bp, synthetic construct
  source 1..200: mol_type = other DNA
aagtgatgacaacagcgggcgaggcgatgacgcttctcatgacggggatgcggagcctcgcgtcggggtcgccattggcactgatctggttaacggggancatccggcagctaaaggtgaggatcatattaagagaagagtgctgtgaatttgaatgatgacttggtctcatgtaatgcagaggtagtggaaggtgctgg

SEQ ID NO: 74 - DNA, 200 bp, synthetic construct
  source 1..200: mol_type = other DNA
gcgcccgaggggtacggcctcaacgccgggtgcgtcgtcaggtactccacgcgggcgttctacctgcccgtcaacgccggcggcggcagcggcagcggcngatcgtccggtgattacattttctacctcgtgctgacctttgaatttccccgtttcacttggttataaagagaggaaaacgacatcggttagactttgtg

SEQ ID NO: 75 - DNA, 200 bp, synthetic construct
  source 1..200: mol_type = other DNA
aatttacaattagtatttcctaagcttaagttttgctagcttggacactgtctttcaaaaattcatttaggagaagttttattttatacttttattgatnaactgctacggtactcatatgcaggaaattgagaaggctgctgaaagaggagagcttactgaacttgtccttatggtcatctggaatcgacttgatattg

SEQ ID NO: 76 - DNA, 200 bp, synthetic construct
  source 1..200: mol_type = other DNA
atactggtgaaaagcttttccattgagccaaaggtcctaggttagaagcaacctctctgcaaaaatgcaggggtaaggtttgtctggattattacttcgncagacctcactcatatgaaagctgccagcactgggtctgtcgtctttttagggattgcagatggtatgcagtgaaagatccattatgtaatcataatttg

SEQ ID NO: 77 - DNA, 200 bp, synthetic construct
  source 1..200: mol_type = other DNA
taggagaaaatttagagagaactgtgcggacaggctaacgtcgcctcaagagcccccggcgcaaaactaaatagtcgcggcctctccaacggtcatctcntctgtacagccaaaacccaaacccaacgggtcgttcctattcaaaaatcgaactcctacgctccttcggtccttcccctccccatcttcccaaaccgccc

SEQ ID NO: 78 - DNA, 200 bp, synthetic construct
  source 1..200: mol_type = other DNA
gaaacaaaactgacgatatatatacatctgaatgttctttagctgtcgatgttaatttgggtgggttatctagatgaactgatacaatatgcttgtatantgcagctgaggccaaagtaagcttggttgcttgtattgtttatgtttacatacttgcacaaacaaacatgcctaggcttgcaagcagggcgggttgcgga

SEQ ID NO: 79 - DNA, 200 bp, synthetic construct
  source 1..200: mol_type = other DNA
atcctattgtattatttatattcagaaaactaaaaaatttggggccaaaaatttggatgccctgtgctattgcccatttatgctgcacagtagacgagcntgcaagtgccgtactgtcgtgacgacgataagcagggcggggcgccaggaagggggcatccgaacggactggcttgagaagcgtcagaatgaatgaggac

SEQ ID NO: 80 - DNA, 200 bp, synthetic construct
  source 1..200: mol_type = other DNA
gatgggtgcattagagagttcggtgagtatgcaaaatttatgcactcctttcaatggttagtattagacatgatgactatatataagacccccactggcnatttgagaggcgtaggagctaagaaacatagctagtgaattgagactccattcatcttgaccctcaaatgttcaaagatcactcggtgattggcataggt

SEQ ID NO: 81 - DNA, 200 bp, synthetic construct
  source 1..200: mol_type = other DNA
cgttgtattagtgcttgcttaggttatgcctagcgattagaagtgatgttatatatcataaaatctttggtgcttgcacgcactattatagttgtaccgngagggactgttaatctggcaaggtcgcaccaactgcgtatagtgtgaccactactgtgtattggagggactagatctgcggtaatttagccagaagctcg

SEQ ID NO: 82 - DNA, 200 bp, synthetic construct
  source 1..200: mol_type = other DNA
taatttagccagaagctcgatagtggagacgacaaggagcatccaagagagaccgaaaccgtatttcacttgcacgtgaagaagactcaaggatatctangaagttatatgatcgaaagcttaaccttcatgtggggctccaatggaaactagtaaaaagcttgtgtgcttctccatatctcgataaaagtatctacaca

SEQ ID NO: 83 - DNA, 200 bp, synthetic construct
  source 1..200: mol_type = other DNA
cggttcagggattggacggggtgttacaaaaggtttctttttagggcctctagctaagttgataaggtggtttgagtagtcctccacaagtcctgagttngttcagaggttttctcgacatgtgtgaaaagatctttttaatgtaaaatgctcgggacatgtgtgagaaggatcttgttttctatatttttaaaaaacaa

SEQ ID NO: 84 - DNA, 200 bp, synthetic construct
  source 1..200: mol_type = other DNA
atgtcgccttggcggtgataccgatgcaaaacgccttggaggtgagtctgggggagtcatcctcggtgtgctgttgtatgattgcaaaaatacatgtggncggggtggccgcctaggtgctgcaggtacgaaaaggttctgtgtgtaaaacatcattcaacagaattttcgtttcatacaagattaactatatcaaatgg

SEQ ID NO: 85 - DNA, 200 bp, synthetic construct
  source 1..200: mol_type = other DNA
agaaagtgttcgccaatgtgggcaagactatcgggtacaaagttggaacaatgattgagatccccagggcagctctggtggctgatgaggtagggaaaancaccaagttcagaatcgcccagaactttgcgaacaagtttgtttatctgtgcattcctacgctggtctgaaatctgtggctgttgttgttgtttttttgg

SEQ ID NO: 86 - DNA, 200 bp, synthetic construct
  source 1..200: mol_type = other DNA
cggtagcaaaaatgttcactgttttctttgaatgctttctgcagggttccgattgctaggctagctgcagctcaggtgcttgtctgaggctgcctcctcnttggcaaccggattgcctgctgctggtggatgtggtgatcaacagtattattacagagccatgctatgtgaacattactagtagcagtgctcataaaagc

SEQ ID NO: 87 - DNA, 200 bp, synthetic construct
  source 1..200: mol_type = other DNA
tgggtttctacctgctgtccaacacggaccactaccacgaggtcatggaggagatcggcgacttcctcgcggctaacctctagcagcacatgcacacaancttcacatgttatagcaagaagatgggtggtaagcttgactttatagttgtaccgatctgcatctgctgctgcttggggtggtggtggtgggctttgctg

SEQ ID NO: 88 - DNA, 200 bp, synthetic construct
  source 1..200: mol_type = other DNA
aaaatgaagtgaatatacattaaggataataattttctgtaaaagttgagtatcattacaaataaaatcacatctatttgatgtgcctaataatttcttntatttcttctggaatgttgtctttggattcattgtccttgtacttcaataattctactaagaatttggtcctcaggacattcccattctgcgcattcatt

SEQ ID NO: 89 - DNA, 200 bp, synthetic construct
  source 1..200: mol_type = other DNA
ataaggctttgcattcaaaaccaaagcttgaatagagaccaggacctgcagcatagttgaattggccgggttccacttctcacatccactgccagtccangtgtttaggaggctaaggcacaccttcccgcaagcatacaagtttggattaagccgcaaaccaccagcacggtaattcaccatctgtatagaaacaaccg

SEQ ID NO: 90 - DNA, 200 bp, synthetic construct
  source 1..200: mol_type = other DNA
tatctggattcgatttatgctgcatttcttccattaggctggtcaaatgatatctggacaggttgttttagtcactgcagctgctggtggaacaggacantttgctgttcaggtgagctgatgacacttatgaactaggattatgcctgttttatgtaatgctccattgcttgtctacttctgcagttattttttattgc

SEQ ID NO: 91 - DNA, 200 bp, synthetic construct
  source 1..200: mol_type = other DNA
aagttcctttgaatattgtgtagggtatgttagaactgcttgcgtctgcttttccgttcttcagctaataaaaaggtagcataaagtcggaatccatttncttaaatctgtcctgtttgccagcgacatcaagatttcgcagtcgagttcgactgctaattacgtattctcttcattttttttatttgtcgcggtttagt

SEQ ID NO: 92 - DNA, 200 bp, synthetic construct
  source 1..200: mol_type = other DNA
tatgccgtacatatatgcagatagtatgacgccaaattatgcactagcaaccttggacttgcgtgccatggcaaccacatggtgaaagcgttgcgcgtcncaagggaccttgagcaccccctgctgcccgtacccgaactgttcctcggccatctccagcagcaccgccatgcagggctccctgaggtctctcacgcgca

SEQ ID NO: 93 - DNA, 200 bp, synthetic construct
  source 1..200: mol_type = other DNA
ggcgcaagcagatgaacgagtacctcgccgcgctccgctccctcatgccgcccgcctacacgcaacgggtaaccgacgagctcgattccgatgatccccntgccagccatgcacgccctttccttgtacctagcaaaaaaaaaaaaaaatcaatctagcacgacgacgacgcaccaggctaatgggctcgcgcgtctgcc

SEQ ID NO: 94 - DNA, 200 bp, synthetic construct
  source 1..200: mol_type = other DNA
acggcagagcacacgcaattagtaggcgtgtgaattctgtttcgctcgctgtgggccactgggccggtcatcattcttctcggctgtttattggcatgcncgaaaagcatatgtatgcacagctagctagagccaagaagcggacaagctaatgtgtatggttcatccattcatgagatgggtgttgatggtgctgggca

SEQ ID NO: 95 - DNA, 200 bp, synthetic construct
  source 1..200: mol_type = other DNA
gcggagtggctggtgctaggcctcgtttctgcggccgcccgacgcctctcttcgccccggcagccttgccgcccctcctggatccaccgcgtccggtgcnctccttccgtccggttcctctcttgcgtttagtcgtctgctcctccatcgccgccggccgctttcgcgtcgtacccaggtgggaccccgacccccgcttc

SEQ ID NO: 96 - DNA, 200 bp, synthetic construct
  source 1..200: mol_type = other DNA
aagttgagggcggagcctgaagtaaaggtccatataacagaagcaagtataaccagtagagatatgaacatcagcctgccacctctggtaacctgaatantaggggtgcctggttcttcccctgttgcttgctgtgacctgaagctagagatgtgctgaagtgttctgttgataaaagtgttttcttcacctgcagtttg

SEQ ID NO: 97 - DNA, 200 bp, synthetic construct
  source 1..200: mol_type = other DNA
gtttcagctgatccaatgagtgcaatttttttttgtcattccagtaacccaaattaaaattcataacactttgtccacaataatgtcatatctctaattngtccagtaaattctgcaccactgctgcttgctggtttttcagagatgctttaaaaagatgcccaaaaggtttggatcataactgctgaattcattgagcc

SEQ ID NO: 98 - DNA, 200 bp, synthetic construct
  source 1..200: mol_type = other DNA
ccaacaacccaaaagacacttaacagacaagttggcaagcagagcacacactttgacatcaaaccatgaaacagagccccgtcgccgatcagccttggcnggcagacaggtttcctttgggatttcccgcgaaccggccgatcgttagggggcggaggtctactccggcctcggccgccgtgctcgcctccccgtccaag

SEQ ID NO: 99 - DNA, 200 bp, synthetic construct
  source 1..200: mol_type = other DNA
ccccccccccccacctccaccaaaagaaaaaaaacagtgggttttcccaaggaagttcttggcaattgcaagcctgccatttgtagcatatcttgctcanatgctttcggttccatctaacgcaacgaactcccatgcgtgcatgctttggacaaggatgtcaatgaacaagtcgtatcgtcacctgctttgcctgaact

SEQ ID NO: 100 - DNA, 200 bp, synthetic construct
  source 1..200: mol_type = other DNA
ctgtctggcgaggggcgccgcgcgcaagactacgtgtgcgggctgccgcgcaagatccgcaggatggaggagctggcccacgaccgtgccgcccaaaaanaggcccaatctgtcagcatcagctgggtgttcgacaggcccgtccgtctgcactgaaggaaagttgaaggacatacaccaatcctcaactgaagccccaa

SEQ ID NO: 101 - DNA, 200 bp, synthetic construct
  source 1..200: mol_type = other DNA
cccaaaaagaggcccaatctgtcagcatcagctgggtgttcgacaggcccgtccgtctgcactgaaggaaagttgaaggacatacaccaatcctcaactnaagccccaatcactactactactactggaggcgttggaaatgcaccttactagttaaatgctcctatattctaagttttctatatatatcatataaatat

SEQ ID NO: 102 - DNA, 200 bp, synthetic construct
  source 1..200: mol_type = other DNA
tctgtcagcatcagctgggtgttcgacaggcccgtccgtctgcactgaaggaaagttgaaggacatacaccaatcctcaactgaagccccaatcactacnactactactggaggcgttggaaatgcaccttactagttaaatgctcctatattctaagttttctatatatatcatataaatatcatataagtaagcttta

SEQ ID NO: 103 - DNA, 200 bp, synthetic construct
  source 1..200: mol_type = other DNA
catgatcgggtacggcgaccggtacccgcagcggatccaccaccgggcctcgtcgttgccctcgatcagggaccatcctcagccgatggcgtgcaagganggcacgccgtacttcaactcatccggtgcgaatcctaacccgctggtcggcgcggtggtcggcgggccgggggaggacgacgcttacgaggatgaccgtg

SEQ ID NO: 104 - DNA, 200 bp, synthetic construct
  source 1..200: mol_type = other DNA
cagaaaccaggccaacgatgttcattgcaacaagctgcacggaaacaagcatgttttggtggacatcctttacatttcatccacggagcgtacatctaanccgaaacatcaaacaacttaattacgtaatacaagacttaaagcacacatgcgctactgaaggtcacatctcatcttgacctgaaacaaagtatgcctgt

SEQ ID NO: 105 - DNA, 200 bp, synthetic construct
  source 1..200: mol_type = other DNA
attacgtaatacaagacttaaagcacacatgcgctactgaaggtcacatctcatcttgacctgaaacaaagtatgcctgtattttctaacgcctactganaaaccttcaaacgtgccaggtcctgtgcctaacctaggaaagaacagcgttagcctatacatacatatattacaaacttcagcaaatatacagagcaaac

SEQ ID NO: 106 - DNA, 200 bp, synthetic construct
  source 1..200: mol_type = other DNA
aatgaagtgaagtactacctagacagatatattgtacgtacctagcttgattgtcacatacatgtaataataattaatataatacggtgcatatgtgtcngagttctgcagcttagacgaatgtatgtatgcatgcatatggcgcgtggacggacgcacatagacgatcacgtacctgttgccgagcttggcgtggaggt

SEQ ID NO: 107 - DNA, 200 bp, synthetic construct
  source 1..200: mol_type = other DNA
agcaatttcaatttttcccgtctgaagcgagataaccaaattgaagcgcaaggaccaaggagtcgtaatttcgtagcaagtggtgttaattagatgtagnacaaaccacgtgtctactaaacatctcccgatgggggagaaaacctacagagaaaaaaaggaactcccagaactgaaaacctgagcccgatacggcgaca

SEQ ID NO: 108 - DNA, 200 bp, synthetic construct
  source 1..200: mol_type = other DNA
gcccgtagccacgtttgggttcgggctgggtgagtggagatactcgaaccctttgcagcctgttttcgcatccgcggggatcgacgcccctctgtggtgnacttgctttgggtagctgctcccatatcccacaaggtagctaagcttcatcgggttgtctcccaggatatagttagcctaaaatatatatacaacatgag

SEQ ID NO: 109 - DNA, 200 bp, synthetic construct
  source 1..200: mol_type = other DNA
gctcaggagtagcaacttttccggttccaatagcccaaacaggctcatatgcaatcacaacatcggcccagtttgaaatactatctgcaagcagatacangttgcgcatgcatcaacacctcaagctaaatcaatgcctcccaacttcttctctagaacgcccataaaaaaaatcctaagatgaagaaatttcacctgca

SEQ ID NO: 110 - DNA, 200 bp, synthetic construct
  source 1..200: mol_type = other DNA
ctctcctactaccactacactgactggagttgctccacaaagctgcacggctgcaccaaaccctcctctgcctcgcttcaaaacgcccctacccctaccnttgcctagctcaccagatcgatcttaccatggctttcctcaccatcctcctcctcgcgcgcatttccagctccattgccatctcgtccaactacatctca

SEQ ID NO: 111 - DNA, 200 bp, synthetic construct
  source 1..200: mol_type = other DNA
catcatgtgcatccaggctcaaggctcctgttttagcacgatatcgatgatgcctatggcggcctttggctggtaatgctcatcagtgtcgacttcttcnatctcctgttcacataagcaagcagacaaaaacaaaatctaataagcaactattatagtgatatcacccaaatcaaaatgcatcaacagcctgggccatg

SEQ ID NO: 112 - DNA, 200 bp, synthetic construct
  source 1..200: mol_type = other DNA
atcattttttccactagctttagctttcgttatccattcttgggaagctccatcaaaatcaaaatcacctccttgaattacaaaattttttatgatatgncgaaatggcattcctttgaaatgattactcttgctgcgcaataggataaatattagataatataccaatggccaaaaattaataatagctcatcctgaag

SEQ ID NO: 113 - DNA, 200 bp, synthetic construct
  source 1..200: mol_type = other DNA
tactctcagtggatacattctatgcagaagttgccagtgaagctgtgaaaagaggagctcacatgatcaacgatgtatccagtggacagcttgatccaanaattcttaaagtggcagctgaactgggagttccatatgttgcaatgcacatgaggggagatccgtcaactatgcaaagcgaacaaaatgtacactatgat

SEQ ID NO: 114 - DNA, 200 bp, synthetic construct
  source 1..200: mol_type = other DNA
agaagttgccagtgaagctgtgaaaagaggagctcacatgatcaacgatgtatccagtggacagcttgatccaagaattcttaaagtggcagctgaactnggagttccatatgttgcaatgcacatgaggggagatccgtcaactatgcaaagcgaacaaaatgtacactatgataatgtctgcaaggaagttgctttgg

SEQ ID NO: 115 - DNA, 200 bp, synthetic construct
  source 1..200: mol_type = other DNA
acaagggcattttgagatctactttctgcccatgtcttctaggaactctgaagttacataagaaatcccctgagaaatcggatctggtttcaaacaggcntttacagaatgttgccatacaaactgctcaactgtggttccatatgtttgtgagaaaacggtttgagtacaagaaaactatgtagaaaattttgtgctaa

SEQ ID NO: 116 - DNA, 200 bp, synthetic construct
  source 1..200: mol_type = other DNA
ccggctgctgctggggcggcggccatggagcgcggcgtgcacgcggagcagagtttgtggctctcgttagcgcctctcacgcctggtggaggagtgcccngtgggaagcgcctctcgttcgcgcctctcgcgcgtgttgatctattgaattgttaggctgtctccagcaacgtcctctatatgtattctctatatccgtc

SEQ ID NO: 117 - DNA, 200 bp, synthetic construct
  source 1..200: mol_type = other DNA
ccgacaagccgaagcacaaggccttcgtcacgccatggaagagcagatccaagaaggaccaggacgtgaacagcagcgcatcaaggccgccgctggcccntgtggaccataacaacgccaccaacgcgccagtcttcatgaccgagagctcgccgtcggaaacggttccctccgggaaggccgagaaagggaagaggagg

SEQ ID NO: 118 - DNA, 200 bp, synthetic construct
  source 1..200: mol_type = other DNA
tactatatactgcaagttacagtagtatatgatatactttaagagaagtgagaacaggtgttagcgccaaatcacttgttaatcgtgcagcagttcgacnggatttaatcaacccttgtttaagatacgagcctgactagctggtgactaattcccttctctgtctctgatatgcgagaataggatcgagtaagatatga

SEQ ID NO: 119 - DNA, 200 bp, synthetic construct
  source 1..200: mol_type = other DNA
cctgcatgcagggttggtttgctatggacccggcggttgctcagaaaccatgaatcttgatgtgctccttcttcacaatgccggcctgccaggacaacancaggaacagaatcagccgtctttatacatacgttgctacatgtctctaagcgcacaaggtgttgcaccgaatggtgcgtacctggacgaggaaattcgag

SEQ ID NO: 120 - DNA, 200 bp, synthetic construct
  source 1..200: mol_type = other DNA
agcctgcagttaggatgtcatagcagacgccttagatcttaggcctatggcatcctcgaatcgtccatggtgtccatgaggcgtgactaaggcatccatnggcgattaggctacagatttggacaccataagaagtgagtacagcatccaataaccctagatcaagcaacctaggtcatttgactccgccggacttccct

SEQ ID NO: 121 - DNA, 200 bp, synthetic construct
  source 1..200: mol_type = other DNA
caacaatagagcattagctaacctgtagtgtttttgccaaagcctcatcttccataatctgtcgaatcctcttgactgctgcgccatgttcctcactatncctcttacccttcattaagtcgctcaaatgcattggtacatgttcatcacccatgtccacgtacttcacgaaataatcaccaggaatcagttctaggaca

SEQ ID NO: 122 - DNA, 200 bp, synthetic construct
  source 1..200: mol_type = other DNA
ttctatcaaaatacacgctgtgtttcattttgttatcacataacaaaaaaatccaaaataaaaggtgcactacatgaacctacctatgaaaggtccctgnttttttacgaacagttatccggtgatgaatttcagaaagcttagagtcaagacctggttgtgatatctccaacccttctgatctaactatatttaagtat

SEQ ID NO: 123 - DNA, 200 bp, synthetic construct
  source 1..200: mol_type = other DNA
ttggatcgcgatccattaccacccctagccataccctcgtaggtcgagttgttttactagtagctgcctgaaccatgatttagaagtcgaataagcaggnatctgatgtgtatgcctttactctttttttgttattgctagggaatggtaagtttgataacataaggttggtgtcctgtaataaattacacaataccttc

SEQ ID NO: 124 - DNA, 200 bp, synthetic construct
  source 1..200: mol_type = other DNA
ttagaagaaaacagagttaggatgcctttttcttttgcctaataaagagagagctttacgaattactcaagaaagaaatacaatctgcagcaactagatnctcgacacacactggacaattattgcaatctgcagtatgaaacatccattttgctagctgggtgagctcgtgagtaactgaattctgctccttttgtgtg

SEQ ID NO: 125 - DNA, 200 bp, synthetic construct
  source 1..200: mol_type = other DNA
gtttgctagttggtactacgaagtaggagtatatcagcctatggttccgaattcaaggcaaccaaactctgctgaagtgtaaactgcagagcacaagcanatttcttaaagctgagctgtcatgagttttgtcaacataattgtacgcctggggtagcataatttcttaaaccacccgttcgtttgccctggaaccagct

SEQ ID NO: 126 - DNA, 200 bp, synthetic construct
  source 1..200: mol_type = other DNA
gtccactccagttttctgtaggggagagcccagtaaaggacaggggtgtcccaagaatatttttccccagaaaaaatccaagttagtaggcatgcacacnctggtaattagatcagatccggatgcaaatagccaaatagcttaagttagggttcgtgtgctgggtctcgcacggtaggatgggaagagaagggcgtacc

SEQ ID NO: 127 - DNA, 200 bp, synthetic construct
  source 1..200: mol_type = other DNA
atttgagcataattatccttttctagagctattttgtcctacatggaacaaatccaaacaacaacaaattgaaaattgagcagttttgtcccaccatacnccggaaccaaacaccacctaagtggtttgaatatatacatgtattgtggggtactcaaacaagtacatctgaagttttgtttattttgaagataaatagc

SEQ ID NO: 128 - DNA, 200 bp, synthetic construct
  source 1..200: mol_type = other DNA
gctcatattgccattgtctacaattcatggtctcaggataaaattcacaatcaataggccttcaaatgttgatgcaagtgaaatcgggctgccatggganaagacttacagtgacaatcccttctgaacgaaaggtatcagcctgacacagccaaactggatgcccaattcacgcacatccaaagtcgtcctacaagggc

SEQ ID NO: 129 - DNA, 200 bp, synthetic construct
  source 1..200: mol_type = other DNA
agctaatgaacttcgacttgtctttgcagaaatctacacatactgccattttggattttgtttgagagtgtgatgacattgcatcggatgagggctgcgntgactggactgctggagctagaaggattcaaccagtggattgtgacaaagaaggtggggaatgatctcgaggacactgaagttcctttgcttcagaaaac

SEQ ID NO: 130 - DNA, 200 bp, synthetic construct
  source 1..200: mol_type = other DNA
gaaggaaactgaacaggcgagacgatacggtttttttaggctgcttgcttcgctggaagaacgaggagacggaagggccgggaaagagggctatataggnggcgggcggcatcgtcgggccgtccgatttgtggcactctccatcggcaccgtccaggctgtgcaagaacactctggagcgggaccgacacgtagccgct

SEQ ID NO: 131 - DNA, 200 bp, synthetic construct
  source 1..200: mol_type = other DNA
atgttgctgttattaatgatcttttggcgaaactggaagaatatgccaagggcctgccaccagggttccaaatgaaacctatccaatttgagaaggtaantattccaaatgaaactcaatttgagaaggttccccaacgtgcacttttcttttattaggattttgtttgttcgagtcagatggatatgaatttaatagca

SEQ ID NO: 132 - DNA, 200 bp, synthetic construct
  source 1..200: mol_type = other DNA
ttcctctttccagagagctagtatagtaccgatcgtagagacaagggaacagccattaaagaacacacgaaggcagcaagggctagcaaaccactccttntagccgcttctgcagctttctagctccagctccagctccagctccaggacgcgctttcattcgtctgctcatccgatcgatgcgaaaaccaggcatctcg

SEQ ID NO: 133 - DNA, 200 bp, synthetic construct
  source 1..200: mol_type = other DNA
cgtggacctcccgggggccagggccagggggctcccccaatgcaactacagccgttgaacgatcctttaattttttcccgggaatagtggtagctttttntagttaaattattcggtcaatcaatcggggcccattccctcctccggcggagcccggccattcccaagtcccactcgagttcacttcctcccagcttcct

SEQ ID NO: 134 - DNA, 200 bp, synthetic construct
  source 1..200: mol_type = other DNA
aacaatctcaagctgctcataatgtagtcagactttctgtaggaaatgagaccgagatagtaaagaacatcccggatactgaatttttgggatatgattntctctctgcttctgctgtcattaaaggtctcctagttaatggaaacccagttaatgaagtttctcaaggttctgaagtagaaatattattagaccggaca

SEQ ID NO: 135 - DNA, 200 bp, synthetic construct
  source 1..200: mol_type = other DNA
tactggaagtatagggataggattcatatgaaatgtatatgtaattgtcattggcattctaagcgaaaacacttgtatttctccctaccttgccgtgtangctttctgcagatgacggagcaaattttgtagcttgtaattgtaactagtggccgaacagaacggggaaaagatcaaatcttcacacagcaaaactactg

SEQ ID NO: 136 - DNA, 200 bp, synthetic construct
  source 1..200: mol_type = other DNA
ttccccagaccccgcacagtgcgggaagcctacggcactgggtacgcccttatagtaccacacaaattcttctcttactatgatggtgttaattcacttntattcatggtatgttgctaaaattaaacccacctgttgtgctgcaatttcagccttcagggtaagtctcaaatggttcaggatgaattggcaaggctggg

SEQ ID NO: 137 - DNA, 200 bp, synthetic construct
  source 1..200: mol_type = other DNA
ttaaaagcatgatcccagtgctaagagtagcagctaagtgtaataagataaacaacatatgcatatcgctagcggagaccggagaacgatcttaaaagtnagcgtacaaagcactctcacgccgcaccgagtacctgcgcgtgtccgtagcggcgcggcagagacaacgccgcggggactcgcgacggcgggaagggagc

SEQ ID NO: 138 - DNA, 200 bp, synthetic construct
  source 1..200: mol_type = other DNA
ttgctcgtgcgatagagatcaggattgaggacgacgtcgagaaggagtcctttgtgctagactccatgttgtcggagtccaaaagttgtctttgtgttcnttgcagcaaggatgaagaaggatgtcacatagctcgctacctcgctagcaggtatggttcttgcaattttctccatagtgactacaatctcacagcaaag

SEQ ID NO: 139 - DNA, 200 bp, synthetic construct
  source 1..200: mol_type = other DNA
gccttcttccgctgggcagagaggcaggagggcttcagttacacggctgagggcttccacaacctgattgaggcgctgggaaagatcaagcagttcaggntggtgtggagcttggtggagaccatgcggtgccggggcttgctatctaaggacacattcaagctcatagtccgaaggtacgctcgggctagaaaggtcaa

SEQ ID NO: 140 - DNA, 200 bp, synthetic construct
  source 1..200: mol_type = other DNA
tgaacttcaacttctgttttcttgggtgttctattcttttattttgggtatcctattttggcagcatcctgctgccttgttttttagtgtgggtctgggntttacgttagggtggtactttcagaacattcgtttttgagtgtgggtctggggttcatgttggggtggtactttgagaacattccttttgcctgtggaat

SEQ ID NO: 141 - DNA, 200 bp, synthetic construct
  source 1..200: mol_type = other DNA
tcaatgcatttagtgatctagagagtaacagcattacactagccttccgccttgatgaagatagaaaaagaccaaagctgctctcaagcatggaacggantctaacttgatgaagacagaagagctaggggccttccgccttcgtgaagaaacaaaagagctgtctgaaatccaatcgtcataccttcctgtccgttata

SEQ ID NO: 142 - DNA, 200 bp, synthetic construct
  source 1..200: mol_type = other DNA
attccacagtccttggcttgccattccaccatcagcaacagcaacatcacttgaacatccaggcttaaaagacatgtccggcacaccaaatgactggganctgaatgcagcagaggatagctcgttttccacatccctatgatccctttgtccagaaggtagattgcatataccctttccagacgacatccctcttgaca

SEQ ID NO: 143 - DNA, 200 bp, synthetic construct
  source 1..200: mol_type = other DNA
gttgatgggtttcttttcccctggtttgcaaataaaaacagagagccaaagccaattattagactgcgtcccggatcaagtagagaaaatccatgatcantttcctcgcaattagccgcaagcaacaactagagctctagaagtaactgcaatcaccaggcaggctgacagctcaacggtgatgaacatgaatcatgaca

SEQ ID NO: 144 - DNA, 200 bp, synthetic construct
  source 1..200: mol_type = other DNA
cagctgacgtcaacatcgtccactgcatgctctgaaaccgtagaatgtacctccagccatcagaaacggtcacatgctagtgcaaggtccaaagatgacncccagtcctcgcaaaacaagcagaaggtcgacggagctcgtgtcggtgctggcacaagtgcaccttcttgcagtgccggcgttccaaagtctgatccgaa

SEQ ID NO: 145 - DNA, 200 bp, synthetic construct
  source 1..200: mol_type = other DNA
ctctgcaggtattaaatcttgagcacctttgagttcattgtagcaggcaacctatctgataaacagcagcacatcaccttacatacgccggcaattatgnattcttatacattgatttgtgactgtttttacaaggaacctctacatggtggcaggataagcacttcttacaagggacaaattaaatggttggatcgatt

SEQ ID NO: 146 - DNA, 200 bp, synthetic construct
  source 1..200: mol_type = other DNA
gcaaaatgtggcaggagtcaatgttgataaatgtagaaggcaagctagttcagagatctgatgttcagcagcatggaacgggagccatgtgaacctcacnactttcagtttctgttccattatgtcgtagcgccgaacaaaggcgtgacataatgttgtgttgtattgtattcattcagtttaaaaaaagctgcattaga

SEQ ID NO: 147 - DNA, 200 bp, synthetic construct
  source 1..200: mol_type = other DNA
aaatattatagaaccagtataattcggtacaaattaaaatcaagtgaatacaatagttgtgatcactccggccggtagtgaatgggcattgtcttaatcnaattccacgagggaaaatacattatggcacataatgagtgtgtgtgtgtgtgtgtttttcatgtctatatatgatgatccactgatggaccggcagcttt

SEQ ID NO: 148 - DNA, 200 bp, synthetic construct
  source 1..200: mol_type = other DNA
atgtaggatgatactccagatttattttatttattaggagactgtcacacaggatatagcaacatagaaagtcacatcgatagttactgttgcactctcnatgcttctgacactcttacgcccacaatggtgcacagttcatattctatctctagcggactgattgaagcagcgattgttttatagtttgttgcgtgcca

SEQ ID NO: 149 - DNA, 200 bp, synthetic construct
  source 1..200: mol_type = other DNA
caaactgctccattgttgcggtctactctaccttgaccttgagctgacgattacgaagtcttgatcggttgccttgccattgtgtctctgtctgtgtctngtcgtctccagtgatacgtttatctgggtgcttgtcgcggacggcgtttaggaacaaggactgcggcaagggccggcgatcgcgaggcacagtgggcggt

SEQ ID NO: 150 - DNA, 200 bp, synthetic construct
  source 1..200: mol_type = other DNA
tttgcacgacccgtgcaggtctcttctggtcgctaatgccggtgactgtagggcggttctttcccggtgtggcaccgcagtcgaaatgtccatggaccanaggccttgcagtctcagcgaaaaactgcgcgtagaatcgcttggtggctacgtggacgatggctatctgaacggtctgctaggagtcaccagagcacttg

SEQ ID NO: 151 - DNA, 200 bp, synthetic construct
  source 1..200: mol_type = other DNA
ctgctccagctggtcctgcatctcttcagaagacaacgtttggttcaaagcccttgcagcagagcctcccccattaccgggctccacttgtgcagatgcntcattttcgaccacctgccaatatgcaaagcaaaaaaactcatctcctgcaaggctggcaagggcagtacaaaaaaaccattgtgatgaaattcgaccga

SEQ ID NO: 152 - DNA, 200 bp, synthetic construct
  source 1..200: mol_type = other DNA
caatgccggtatgtggtctggcctctggcccctatagtataaacataacactgaaaaatctgatttagttgtaagctttggagaaattgttgtgagctcnttcatgactaggatctgggagacgtcacccaacaggactcgaacgacttcggtcctataaacagttgaattttttattttttttgtaaagtgtgtaaacg

SEQ ID NO: 153 - DNA, 200 bp, synthetic construct
  source 1..200: mol_type = other DNA
tctaaatatttgacatggactaaagtttagtgggactaccccacgtccaataaaccagcagcaggtcgactgaaacatggcaccaattggattgaacccnagcaaagaccaacgcacattttttgtaccatgcatatctcatgaaacctttgcttgtttcagagcttatttatcaattgtagaagctgttcggctggtgg

SEQ ID NO: 154 - DNA, 200 bp, synthetic construct
  source 1..200: mol_type = other DNA
ttccctgtttccgactcgctgctgcttgaactcacgcgattttcgtggggattgttgctcttttcattcacgtaaatctagctgcggatcgcctgcaccnttttgattgctaatgttgtgccgtagcacgatcggcctgccgagttcatccagattttgggcttgctagctgagcgatttgcctgtgttggtgcaagtgt

SEQ ID NO: 155 - DNA, 200 bp, synthetic construct
  source 1..200: mol_type = other DNA
aactactgtgcttaccgttcagacatgcatcggctcatgctgtactggtactggccgtactgctgtctcttgagcatttcacgttcttggactgaatgtncatcttcgtacctttccggcaatatatttgatccaatcttgatttgccatgtgctgcgagatacttgaatatatttcttaatttaagataaggttcttat

SEQ ID NO: 156 - DNA, 200 bp, synthetic construct
  source 1..200: mol_type = other DNA
tcacactcgaggtttgttgcaccaccggactggtctagtgctcgccacgttagatagtcgttggagctagccattgtcgaccatgagcctctggtccggntgtccattggtgcatcaccaggctagtctggtgtgccaggatcagtattgctgaagaatacgtctctacgtactcgatctggcatgccctctatgtcgtg

SEQ ID NO: 157 - DNA, 200 bp, synthetic construct
  source 1..200: mol_type = other DNA
cggactggtctagtgctcgccacgttagatagtcgttggagctagccattgtcgaccatgagcctctggtccggctgtccattggtgcatcaccaggctngtctggtgtgccaggatcagtattgctgaagaatacgtctctacgtactcgatctggcatgccctctatgtcgtgctagacttgtccgatgtgttaacgg

SEQ ID NO: 158 - DNA, 200 bp, synthetic construct
  source 1..200: mol_type = other DNA
agattaattaggggtacaggacctgtgaaagtctaagaggcaaccacaagcaacaaaagtagatccaccaaaaacatatggcagttgagtcacgtcatgnaccaaactatacaactgaaaaggaaaagggaatccagctgctgctcctttgcaaatgacatcttctagtttcgcataaatagatttatgagaactgacaa

SEQ ID NO: 159 - DNA, 200 bp, synthetic construct
  source 1..200: mol_type = other DNA
cataacagccctccaactgctcttgtcccgtgcccatgccttcagttcagagctttgctgacttctcccttcagcaaaatgagtcctatggcttgctctncgttccctccggtgatggctgctgtttttggtagaggagtcaaaactcttcttgcagttgtagatatgagatgcatgcacattgggtaacgactgccgta

SEQ ID NO: 160 - DNA, 200 bp, synthetic construct
  source 1..200: mol_type = other DNA
ttcataattaaattagtttaggtctcgccctgtgtgacttgcttccaatagaggacgctagtaaccattctgatctttttgccaaatagtagtagtttgntgccaaaatcaccatcccgaacgtctcacacaattcaaattgaagatggaacaactgtttcttgggccttggttctcttaggcctccacatggtccgggc

SEQ ID NO: 161 - DNA, 200 bp, synthetic construct
  source 1..200: mol_type = other DNA
cccatcgtcggcaacacacgctttacgaaggtgctcatggatggaggtagcagcatcaacaacatctatatgaacaccttggagctcatgggggtcaggngatcccagatccggcccagggtcgtgccattccctgggatcaccccggcaagagggtgcatccccttggacagatcgacctacccatctgctacggaacc

SEQ ID NO: 162 - DNA, 200 bp, synthetic construct
  source 1..200: mol_type = other DNA
ggttttaaattagaaatcaaagaagtttcaaaaaatgaaaaaagagataaaccgggcaattttgctgattagccacccttgttagccgattaatcaaccncggataaagataaaccgggggcctctagtagctaggtttttgggattttctcccttttaacttcaagtctttttaacattggtgttgctacttgtaataa

SEQ ID NO: 163 - DNA, 200 bp, synthetic construct
  source 1..200: mol_type = other DNA
agatgtacggagagagcttccctggtggtgcaggccggcgcgagctcgactcctacgggtgcaggaaccaatcacgccgatagagcagcttagggtgcgnatcttcttccctagacaccagcagaatccattgcccaatttccattagaaaaccttaggatagctctagctgacttgagatgttattgcttcaggactga

SEQ ID NO: 164 - DNA, 200 bp, synthetic construct
  source 1..200: mol_type = other DNA
tgacattaatatcagccactcctctctctcaaaaaaagacgacattaatacaactgctcctgcatcgttttcccactgattccaggcgctacagccaacnggagtacaggacggaacgagcccggttagtgttcgattagaaaagagcaaaggaatactcgcgcatcacaagcatcgcggtctttgtggctactagagta

SEQ ID NO: 165 - DNA, 200 bp, synthetic construct
  source 1..200: mol_type = other DNA
tgttacgaagtttaacgtacaagaatataattttaatgttaaggccatggataaccaaaatgaattatttgaattgggtggagtctccatggacccggcncgaaatctgaactcaaaactcgaaaccgctccaaacaccgtttcaggtgataatcgaaacccggaccaaaatctgctggatcccaaaaccctgattggtt

SEQ ID NO: 166 - DNA, 200 bp, synthetic construct
  source 1..200: mol_type = other DNA
tgcagctttgtacaaagacagcttcattttatacttgaaactaagtgacatcaaaagagtgatgcatcctataaagcttatgtggaaatatggttcaagngaactcgtgccagaagctctgctcaagccaccagctatttgagttgacaaactctggtcctttttttgagggtggagcaagctttgaatcagatcaatgg

SEQ ID NO: 167 - DNA, 200 bp, synthetic construct
  source 1..200: mol_type = other DNA
tttgtacaaagacagcttcattttatacttgaaactaagtgacatcaaaagagtgatgcatcctataaagcttatgtggaaatatggttcaagagaactngtgccagaagctctgctcaagccaccagctatttgagttgacaaactctggtcctttttttgagggtggagcaagctttgaatcagatcaatggcttgaa

SEQ ID NO: 168 - DNA, 200 bp, synthetic construct
  source 1..200: mol_type = other DNA
aggccggtgtctcagactcagagacgcccttgctgtctgatgatagaataattgaagctttccagtcgcgaaagcttgaggagatagacagggcccagcnacccagatggacaagatacatgcatgcatgcatgcaacaatgaaatcactcggctcatctcgcgtcctggacctggaaactgtagcactccactagggta

SEQ ID NO: 169 - DNA, 200 bp, synthetic construct
  source 1..200: mol_type = other DNA
tctttgggtacccaccggccaccgcaaacagtaatttgcctctttttatttaattacaaacgagtcacggccctaccaaagaacacgatgaaaagaagcntacactgtcagattctagtaaaaagaagttctgctactctgtttttgaaaatttgacaaaagattaggaacaccatcatagatctgttctttttgtcccc

SEQ ID NO: 170 - DNA, 200 bp, synthetic construct
  source 1..200: mol_type = other DNA
actcgagttctcatacataatttgagtaaccatcaatgttttgctatattatttctaaataatggagaagctgggaactctactagatcggcatatctanactctcgtgggtgtttacgaaaatgtggctgacttaagggcgtgactaccattttcgcaagaattgagcccgacagcaaaaagaaaggtgacgtttctct

SEQ ID NO: 171 - DNA, 200 bp, synthetic construct
  source 1..200: mol_type = other DNA
cgatgctgtaaaattggtggagatcgagcacacagtgtcagcgtcgacgacgtcaattccacgcaggtactagctcaccagtatcggttccatcttcagncagatacctggcttgagcgcatcgatcggttgcatacaacactagaatgaacactactgtggatagtcttggagaagctagctgagtgagcatgtttgac

SEQ ID NO: 172 - DNA, 200 bp, synthetic construct
  source 1..200: mol_type = other DNA
ccgacagcggcaacaccagtggagttcttacatgtgaaggttggggcaaagaagcgagggcggcccaaaggcagcaagaacaaaaaagctgtggttgagntaaattagggctatggaatgacacatgtgaaggttggggcaaagaagcgaggatgccctgactgcctgaaggcagcaagaacaagaaagtggtgattgag

SEQ ID NO: 173 - DNA, 200 bp, synthetic construct
  source 1..200: mol_type = other DNA
ttgtccagggcgactgatagaagatgaggggatgggactcggactgcgactgggactggaccaggtgttacgttggattcttgcgtgattatacaacaancaaatgatatttaagctgaggcagtgttagattcctcacaaacgagatctctgtgtcctaaagttttggctccccgttgaattgtgtcgctgccgcacct

SEQ ID NO: 174 - DNA, 200 bp, synthetic construct
  source 1..200: mol_type = other DNA
ccgacggggacggcgggtggttgtggtccacggcgaggcggactccggagggagggtggtggggacctgaaagcagaccgctgctcgtgtctgaattctnatcagcgaaggaaatatcaggtacgggagagatatttctgcaagagcgcaactcaaactcagttagctacccacagatgagaatgcccgtttacttattt

SEQ ID NO: 175 - DNA, 200 bp, synthetic construct
  source 1..200: mol_type = other DNA
gtcagaattcagtgacgagatatgtgccgagaatggtgctgaagaggacgatgaaacagaggatttaatttggagcaaagaagagatcgatgctatttcngcgctcttcgaccggccgatgcgccagaagcccctgaagccgcgaaaccccgcgaggcagagggcgcttccattgccactgccgcacaagacgaggctgc

SEQ ID NO: 176 - DNA, 200 bp, synthetic construct
  source 1..200: mol_type = other DNA
aggagtaagctgtcacggataagctcgcaggatactttatcatcactggagcctgcaaacagtcagtgcacaaagaggacatgtgtaaatctgcatgagngatcgattaacatagacagtgaagtcctctcatcttcttgctggcaagaacacacacacagaaaaagaggaaaggggctcgctcgggattaatcgcaagc

SEQ ID NO: 177 - DNA, 200 bp, synthetic construct
  source 1..200: mol_type = other DNA
tacacatggtgtaggtctgcaaagcagataaagtagaattccctagagtcaatcttcctgccatcgaaagcagccataccctgaccatgttcaaccgtcnttcaattttgttcatgagggggagaaaatccctgactttaggttcagtagggtacatgttttcgttatatacctttctaaagtgtgcgcacacaaatgac

SEQ ID NO: 178 - DNA, 200 bp, synthetic construct
  source 1..200: mol_type = other DNA
cagcatctccaggtaccgccgggtctcgcgcctctccaggttggacacgtacttccagaacccgtacacgccggtgagggtcatcagcctctccgagtanagcttccaggtgtacctgcagtaccgaacgcgacgaccggtaagaagcacatcaaaaaaaacggctggggaaaccatcttcacgtcagtgcatacttctc

SEQ ID NO: 179 - DNA, 200 bp, synthetic construct
  source 1..200: mol_type = other DNA
cgcgctcagcctcagggatcgcattgttgtactcggcaatgatctggtggggctgcagcataccctttccaaggtttttcagcctgcgcaaaacgatgtnccaaataacatcagactatgccagatctataaactcatcaaacatatacaatttcaagaaatagtttagacgtatgatcagcagtcagtagcgtgggaac

SEQ ID NO: 180 - DNA, 200 bp, synthetic construct
  source 1..200: mol_type = other DNA
gtattcgatagaccctttagtaacaagctatacattgggtaagttcagactccaatcattctgttcagaaacatcgtattgaatataaaactaaagaacncacatgcaggtgcagccagatctaacagcagtttacagtcggtactaaaaaaagcatggtgtatgtatgtatcatcagtatccagtactaggtttcgaca

SEQ ID NO: 181 - DNA, 200 bp, synthetic construct
  source 1..200: mol_type = other DNA
gtagcaaaataaatgtaaacttgcagacgaagcgggggaaatgagggcagacctggtgaagacggcgacaagctcattggggtgggcagagagtgagtcnccaatgcgctccctgacgctgtggaggcggctcaggacacggtcacctgcaccttcccccattgctgtcctcttcctggatcctcaggcctgcacagcga

SEQ ID NO: 182 - DNA, 200 bp, synthetic construct
  source 1..200: mol_type = other DNA
agcgtgacgcggcagggtacttctctagaaaagcacggcatcagcaaggtgggggggctggggttccttattgcaggcaatcacgaggtgattagcacanacggaagtaattacacctagatgatgataataaggcgcaggattaacgtaagttaaccatggccattagcacaagaacccaggactgtcagcctgtctcc

SEQ ID NO: 183 - DNA, 200 bp, synthetic construct
  source 1..200: mol_type = other DNA
gcagatcgccaactgctgcaaggccggcgtggtgtcggcgtacgggcaggacccggcggggtccgtctccgcgttccaggtctccgtcggcctggccggnaccaccaacaagacggtgaagctgcccaggaacttcacgctcatggggcccgggctgggctacacctgcgggcccgccgccgtggtgccgtccaccgtgt

SEQ ID NO: 184 - DNA, 200 bp, synthetic construct
  source 1..200: mol_type = other DNA
cgagtgcaagatgccgccgccggactcctacccctacctgcccaacgccgcgcccgtcgtcgcctcgcagctggtcctgtccgccgccgcctcggcgttnctactgttgctgctcctggtggcatgaccgtgaccgaaccaagggcaaggcctccgttttgttttcccgtctcgtcccgtgggcagggagcagacttcag

SEQ ID NO: 185 - DNA, 200 bp, synthetic construct
  source 1..200: mol_type = other DNA
gtctgccgccactttgcccttcgctgtgttgccctcgcgtgcgttgcctcggcatattttccaccccacggaccgggtctggttcctaggccggtgcgangaggaaaggccaggatacggctcactcagggcttaacagaacacaatttttttggaccgggtttctttcgaggcgggttttggccttgatcccggccagg

SEQ ID NO: 186 - DNA, 200 bp, synthetic construct
  source 1..200: mol_type = other DNA
ggcaatggagtgtgaccgcttgtgtccaccgagcgcctgacccgagccaaacactctaaaacagatgggacactcgaatttgatcgctttgtgtgacttngccttcaaggccaccaccgctgtagtagccatgtgagtggtgtagttgatgttaaacgaggagatgttggtctcgacaattcgctccggtcgttggtcga

SEQ ID NO: 187 - DNA, 200 bp, synthetic construct
  source 1..200: mol_type = other DNA
gatcgcatagagcactatgaggaggaagatgaacaccaggggaaacttggcggtcaggtgaaccaggaacgacaaggacttgcgcagcgaagcttgtacnctctctgcgttcagcagagggacagggaccccatggttggcatgatttgcattggtgggcgaagtagtgtcaaccggcggtggcagcggaaggtggcgtc

SEQ ID NO: 188 - DNA, 200 bp, synthetic construct
  source 1..200: mol_type = other DNA
gactctctcgatttggcgcatctgtgaggttcggtttgtctctgacaggtatggttcgcttcctctagtgtttacggttctatttgctctttccatgatncgattttcaccaacactgaagctgctctcaccttttagtgcattgcaagagttatgtgtctagaaaagccaaaaaaacatcttataatttggaatagagg

SEQ ID NO: 189 - DNA, 200 bp, synthetic construct
  source 1..200: mol_type = other DNA
catttttgctgaggcaatctagaactgtgttgtatgtaattaaattaggtggataaatggtacgtgtcatttcttcgagcagctgcattacctccttaantttgccttctcttcctaatcctgacaaaagtgtgttgtacgtaccattagtgggctcaattttcatttctttcaatttatggaaaagttgccatgcttcg

SEQ ID NO: 190 - DNA, 200 bp, synthetic construct
  source 1..200: mol_type = other DNA
ctcgttcagcaaccagagaacagtgtcgacatccctcttcttcccaaacgccaccatcagcacggagtaggtcctcacactcggcgagatgccgtcctcnaccatcgccttgtaaacctccatggcctccgcatcgaagccggacttaacgaggaagtaaatcaagccattgtacgtgtaggcgttgagggacatccccg

SEQ ID NO: 191 - DNA, 200 bp, synthetic construct
  source 1..200: mol_type = other DNA
catttgatgcggtcacaacattgtcatatagttttattttataattgcctcagcaagcagaattgatggcagataattttgtttgctaatctcaaggatngagatatttgagcttgggtagaaaaataaaaaaattaggtatatccatttttgaagtttttgatgctgctgcagcctgcagacttcaaacaacctcaaag

SEQ ID NO: 192 - DNA, 200 bp, synthetic construct
  source 1..200: mol_type = other DNA
gatcatatgtgtacataatctacataattcacaccaccattagcacacaccaacctacataattcacatcaccattagcacacaccaatcgctgcaactnttttttagttgcttctggtgtctgtaagccatcgtatgagagtggcctacagcagccgttgctgtaaagtagggctaccgaacaacaacctctgtcttta

SEQ ID NO: 193 - DNA, 200 bp, synthetic construct
  source 1..200: mol_type = other DNA
gatcccaggagcctgtcacgggccaacacctgtagttcaatggaaatgcagaagtgtaaacatgatctatctgtgcaactagctatatcttggtggccantgaatcatgtttgtttgttttttgttcacctctggtggcttcacccacatctggccgtcgtaccatccggtttcttcgtacggtaccaccgctgacagca

SEQ ID NO: 194 - DNA, 200 bp, synthetic construct
  source 1..200: mol_type = other DNA
tttttatatgaggtcaaaagatcaatggactttttactatcaatattctattactacggtgggttaaatactgtttcaagttatacttatgacgcactantactctacagttcttccatcgttctcaggaaatgtcccagctattttcgcaaaattatttccgtcagccaacattacgcgacttggtgattggtaccaca

SEQ ID NO: 195 - DNA, 200 bp, synthetic construct
  source 1..200: mol_type = other DNA
tttggttgaatgaagcacgtgtctatataaattattattacggctggtgccgtgcttaattaggtcgccatatatatgtagctgcgccgtattttacagntacggtgtatccgaattagctagtgagtctcgtgtggatcggagccacacgaccggtcgacgaggacaatattgttaatttgggagtttcagtttaattt

SEQ ID NO: 196 - DNA, 200 bp, synthetic construct
  source 1..200: mol_type = other DNA
cgacctcgctggcctcgccgccagtcccgaaattaagttcaccgaaccacaggtccctgagatccccaaccaccgttcccgcactgcatccgctgggtgncatcagcttcatgcgctcacagcgtgcgaatggaatgcaggttaagtgctatatgcatcagctgctgtcgggtctggagcactgccacgaccggggggtg

SEQ ID NO: 197 - DNA, 200 bp, synthetic construct
  source 1..200: mol_type = other DNA
tcggttcacgcactttggctttagtaacgtagagcctgcatgctttcggttaattatcatggtagtaggtcttaagcgccagctagctgcaacaaatctngtatcgcacacgctggatcagcaaaggtagctttcgagtcatcccattgcgtttgggcccggcctcctcccactcacgctgcatccgtcttgcttccttc

SEQ ID NO: 198 - DNA, 200 bp, synthetic construct
  source 1..200: mol_type = other DNA
aaaatcatgaaacttttcaagtgaagtagtgaacaacaactataagcaacgtccgcaagcgcaatccacagcttgaggttacattgtgagtgtagtaganaaactaggtggtcccaaatagcacaagattatccaagctacccagttttctacgattatgggcacgaccataagagcatagcagtgcttcgcatcctgtt

SEQ ID NO: 199 - DNA, 200 bp, synthetic construct
  source 1..200: mol_type = other DNA
tgccaagctgatgctgagccttattgtgtgtggatccgttttctgctaattcagatatatgccttttctcaaaggccggcagatctaggggtataacttntctcgaacactggcagatccttgttacacaatgattgatagcattggacaccagcagcgaaagaaatctgtaataaatcacatgtacggacacgtacagt

SEQ ID NO: 200 - DNA, 200 bp, synthetic construct
  source 1..200: mol_type = other DNA
cgtcacggacctgcgtcacttaccggcgtgtgtaggcatacaccggatggcgtatttgtggcgtcctttctagtatgcaggtcactgtagaaactcctangctgaggtcccaacgaccggggtggactggccccatacgtaagtgtgggtttgcctgggatagtacgtgacgacgtaacgtttgatgttcagcagggacc

SEQ ID NO: 201 - DNA, 200 bp, synthetic construct
  source 1..200: mol_type = other DNA
ggcggaggtggaggatgcgggtgccgggcagctggacctgccgccggggttccgcttccaccccaccgacgaggagatcatctcgcactacctcgcccanaaggccctcaaccaccgcttcgtctccggtgtcatcggcgaggtcgacctcaacaagtgcgagccatgggacctgccaggttagtacttgctgctactgc

SEQ ID NO: 202 - DNA, 200 bp, synthetic construct
  source 1..200: mol_type = other DNA
ttcaaacctctcctatgttgatctttcaagcaattttttgcacggctcactgcctatgggcattggtgatttgagcgcgttgacattgctgaaccttcgncagaacaactttactggcgaaatcccaagaacaatcaccaaacttaagaatctcttatacattgacttatcaagcaacaatttcaatggtagcatacctg

SEQ ID NO: 203 - DNA, 200 bp, synthetic construct
  source 1..200: mol_type = other DNA
actttattgcgtcttgatcggcaaaacgaaatgcctatttatacatttgccttgacacattggattttgtttttctctttcttcttttcaatgttagggntcgctgcattcatccatctccaagccttgctcttcatagggcttttagtttgcaagtcttgaattctgtagatgatgtgttgttggtaatttgaattgac

SEQ ID NO: 204 - DNA, 200 bp, synthetic construct
  source 1..200: mol_type = other DNA
atttttccttccaaaagatgaccaatgctattttcctttgtatgcaggctataccgatccttctaaaccagatcttccccggttgcatgaatggcaattncgatctggactggatcgctacatttggatcgtggggatgatatatgcatattaccatccaactgtatgtttttgaccatatattttttattgtagttgtt

SEQ ID NO: 205 - DNA, 200 bp, synthetic construct
  source 1..200: mol_type = other DNA
tgccaagggatttctcgaggctgtctgcataatgatcatgctgatgaaacactaggcaacaggctgtggaagacatcatgcatatgacaaacgggtccgncatctgtgataacctgctgtggaattatgaagcaccaggtttgtggaagtatcaacttcagcctctatttgcagctctttccctctcccgcttgttatac

SEQ ID NO: 206 - DNA, 200 bp, synthetic construct
  source 1..200: mol_type = other DNA
gtcctggtttagtagccatatcgtcaaccattagacagttacaaccataatgatgtacacaatcagccagacgcacgccagtcactgtacagtacaatantatggtacttcgtttccaataaaaagatggaaacccataggagtcttttaagcacagtactacagtaggataagctgaagcttctaaaacaccaatgcat

SEQ ID NO: 207 - DNA, 200 bp, synthetic construct
  source 1..200: mol_type = other DNA
ggcaactatttctagtgatcatgagacaaatcaactccttaacatggattttcatgaaaaccatcaccaggaaggagatgaatatcatgcccaggattcnaatccatttccctcgtcagaagctgaatgtttggcagaaattgattcagtaaaaagtgaaaatctgcacccattgcagctacctgagcttatagttgttc

SEQ ID NO: 208 - DNA, 200 bp, synthetic construct
  source 1..200: mol_type = other DNA
gcggtggattcggaacccttggcgcccttgatctcacgagtgctaccctggagaactgctgctgccttctgtgacacagtgagctgctgagatccgtggnttccttcgcgtcatcgttgctttggacaaactgcttctccttggagtagctgatgctggtcgcagaactctccagcagcttcttccatagcatcctcttg

SEQ ID NO: 209 - DNA, 200 bp, synthetic construct
  source 1..200: mol_type = other DNA
gcgtcaccgggaatctctcggcgctgggaacgtgaaaggccggaggaggaggaggggtgtcggttgtttcttgcgccgcggacggcgccgcttggtcgcnatccgtggcccgccggcagctggtcttttgcatgcttgttatttcctcgccgggtagctcaaccggaggagctctgcctccgcgttctgggagggattcc

SEQ ID NO: 210 - DNA, 200 bp, synthetic construct
  source 1..200: mol_type = other DNA
tttgaatacattagtcgtataatgcaaggcgcagctcagaataatgggcgtcgtttgcatcaccatcgcccgcgacagaaacacactccgccgaacggtngtgtgcatcgtcgcatctcgcggcaaagcgcacgggccggccattggtggatccggtgggggggccgccagcagccttgctccaccagccgcgatcatgg

SEQ ID NO: 211 - DNA, 200 bp, synthetic construct
  source 1..200: mol_type = other DNA
cagcaggcaaccatgtgatcctctgtcatcagatccaatcactcacatgctgaggtgagcagctgttgccaccaaatcaaaaccaacgcagggtgtttcncatcatagcactgtcagcatggacaccataaaaaatgttcagaccagcgagctcaacagatagcagccataagcatcatcgtgtaaggagagcgatgtgt

SEQ ID NO: 212 - DNA, 200 bp, synthetic construct
  source 1..200: mol_type = other DNA
gctctcccttcatcagagtgttgagcctctgatgctctgtgattgcatcaatacatgcttgaatagctggaaggtgggggttccgttgtggctgttgtanactgccaaaagtttgcgctgcacatggaaaataatgactaagaactaacgaaaatctaagctaaactttaagaagtaggtcaacttcaaccacagcacaa

SEQ ID NO: 213 - DNA, 200 bp, synthetic construct
  source 1..200: mol_type = other DNA
acgctgccgcttctttttctattgtttgacttttgaactccacgaatgaaggatggtggaagagacttggaccttgtcaatttactagtagttgtgtctntccaaccatcattggtgctgatgccaagaggatatgagctttgtattctatcactctgcacttctgactttggacatttcctgcatgtcattttatgggt

SEQ ID NO: 214 - DNA, 200 bp, synthetic construct
  source 1..200: mol_type = other DNA
acctgactcacccatatctcctcttttgacttctgatcctaagaacgagcggtcacattcacaaacttttagcagaccgcagagtgaacaagatgatacnagttctgaacgtagtgaactggatgaggttcagcaccagaaacctccatctttttggcgtcttgcaacacttagtatcgctgaatggccttatgctcttt

SEQ ID NO: 215 - DNA, 200 bp, synthetic construct
  source 1..200: mol_type = other DNA
tatagtccttattactattaagttctatatatagaaagcaataacggctcagtagattaatccaaacaccaaaacttcgcaaactaggagagaacgaacncagcagctagcgactagcttgttaccatatccctaaacacaagcgcatccagaagctcatccgcgcaatcagaacctcatcaactggcatttacactagg

SEQ ID NO: 216 - DNA, 200 bp, synthetic construct
  source 1..200: mol_type = other DNA
tacagtacaaaatgtgctgaatggtaagagcatacaaggcattgctgataggccatgaccaattctatattggtgacagataagatacagtttacaacanattacacttgccttccagaaattcctggaaactagacagagtgctacagcaccactaatccccgttaaaactttttgacaaatcctaaagacactggcaa

SEQ ID NO: 217 - DNA, 200 bp, synthetic construct
  source 1..200: mol_type = other DNA
cctcccctcacctcggcccgcctggagctctctgctcagcgagcacgccatcacagagacgcacatgtcaacacagcgagaaaactgcactgatctgaancccaaatcacttgtttaataattggccgtggcaatcagatgacgtcagccgccagtgcctcgatcccccatttcttaactcccgagaatgagttggaacg

SEQ ID NO: 218 - DNA, 200 bp, synthetic construct
  source 1..200: mol_type = other DNA
caatgcaacaataatgaaatctactctaggattagaataaaaaacaataattaatgtacttagtcgctacatggtacaatagctgtaatcttgtgaaagngcaatccgatgagcttacgaacaaaggactcttgcaacagtctttcctctcaagtagccgctacgaagtcgaataaccatgtgctatgtgcagggccttc

SEQ ID NO: 219 - DNA, 200 bp, synthetic construct
  source 1..200: mol_type = other DNA
agggcaattataagtttcctcatgatttcagttttgaaaaaacatgggttatggtagataagttgagcggccaccgagacattcaaaattcttcaaattntttaccctgctgctactcctattgagttgccagtctatatagagtttgctacaatgcacttgacagagaaatttcctatgtaaatacatcatgttcgctt

SEQ ID NO: 220 - DNA, 200 bp, synthetic construct
  source 1..200: mol_type = other DNA
tgattggatgctttctgttgaatttgcaatcagctaaatgtgactaatgccttggcttctgcacagacagtaagtaattgaagtttctatgcaacagtantttgctgcatctgtctttgttgcatgaacacgacacatgtatcatccctatatcaaggggttcagttatgttctgtcttgtcagtgtggtggccagatga

SEQ ID NO: 221 - DNA, 200 bp, synthetic construct
  source 1..200: mol_type = other DNA
agtgaagttgagtagcaatagatggatttctgcaataagacatgaggaatggtagcaaataagggctccgctgagtccatccatcatccgtgtatgattngctttcttctcgtgttttgttcatgatttgtgtcagaaacctaggtggcatctagtctggtttatgtattgccagtgctgttgtgtagaaaaatctccgt

SEQ ID NO: 222 - DNA, 200 bp, synthetic construct
  source 1..200: mol_type = other DNA
tcattcaacaaagttagaagggtgcaggctcatctgctcccatgcataatccatatatagatatctattattaggtacaaaaggcagcacgaaaatttcnttgtgaaagcattaaaacatgggtggaggggtctgcctctactttagaagattggaatattgcagacttgagtaaaactcttctgataccttaaaggagc

SEQ ID NO: 223 - DNA, 200 bp, synthetic construct
  source 1..200: mol_type = other DNA
caatctacccggaggtctctgctttccaactgagtttgcagtgttcaattcacctcttaaaccaaatcttggtctgcagctaccaaagtgggaggaatcnatcatggaggcaagaagcagatacgcaagcataatcaaagctcttgctgacaaatatcctcatgaaaacctgctattagtaacacacggtatatttccat

SEQ ID NO: 224 - DNA, 2370 bp, synthetic construct
  source 1..2370: mol_type = other DNA
atgaacaagaacaacaccaagctgagcacccgcgccctgccgagcttcatcgactacttcaacggcatctacggcttcgccaccggcatcaaggacatcatgaacatgatcttcaagaccgacaccggcggcgacctgaccctggacgagatcctgaagaaccagcagctgctgaacgacatcagcggcaagctggacggcgtgaacggcagcctgaacgacctgatcgcccagggcaacctgaacaccgagctgagcaaggagatccttaagatcgccaacgagcagaaccaggtgctgaacgacgtgaacaacaagctggacgccatcaacaccatgctgcgcgtgtacctgccgaagatcaccagcatgctgagcgacgtgattaagcagaactacgccctgagcctgcagatcgagtacctgagcaagcagctgcaggagatcagcgacaagctggacatcatcaacgtgaacgtcctgatcaacagcaccctgaccgagatcaccccggcctaccagcgcatcaagtacgtgaacgagaagttcgaagagctgaccttcgccaccgagaccagcagcaaggtgaagaaggacggcagcccggccgacatcctggacgagctgaccgagctgaccgagctggcgaagagcgtgaccaagaacgacgtggacggcttcgagttctacctgaacaccttccacgacgtgatggtgggcaacaacctgttcggccgcagcgccctgaagaccgccagcgagctgatcaccaaggagaacgtgaagaccagcggcagcgaggtgggcaacgtgtacaacttcctgatcgtgctgaccgccctgcaggcccaggccttcctgaccctgaccacctgtcgcaagctgctgggcctggccgacatcgactacaccagcatcatgaacgagcacttgaacaaggagaaggaggagttccgcgtgaacatcctgccgaccctgagcaacaccttcagcaacccgaactacgccaaggtgaagggcagcgacgaggacgccaagatgatcgtggaggctaagccgggccacgcgttgatcggcttcgagatcagcaacgacagcatcaccgtgctgaaggtgtacgaggccaagctgaagcagaactaccaggtggacaaggacagcttgagcgaggtgatctacggcgacatggacaagctgctgtgtccggaccagagcgagcaaatctactacaccaacaacatcgtgttcccgaacgagtacgtgatcaccaagatcgacttcaccaagaagatgaagaccctgcgctacgaggtgaccgccaacttctacgacagcagcaccggcgagatcgacctgaacaagaagaaggtggagagcagcgaggccgagtaccgcaccctgagcgcgaacgacgacggcgtctacatgccactgggcgtgatcagcgagaccttcctgaccccgatcaacggctttggcctgcaggccgacgagaacagccgcctgatcaccctgacctgtaagagctacctgcgcgagctgctgctagccaccgacctgagcaacaaggagaccaagctgatcgtgccaccgagcggcttcatcagcaacatcgtggagaacggcagcatcgaggaggacaacctggagccgtggaaggccaacaacaagaacgcctacgtcgaccacaccggcggcgtgaacggcaccaaggccctgtacgtgcacaaggacggcggcatcagccagttcatcggcgacaagctgaagccgaagaccgagtacgtgatccagtacaccgtgaagggcaagccatcgattcacctgaaggacgagaacaccggctacatccactacgaggacaccaacaacaacctggaggactaccagaccatcaacaagcgcttcaccaccggcaccgacctgaagggcgtgtacctgatcctgaagagccagaacggcgacgaggcctggggcgacaacttcatcatcctggagatcagcccgagcgagaagctgctgagcccggagctgatcaacaccaacaactggaccagcaccggcagcaccaacatcagcggcaacaccctgaccctgtaccagggcggccgcggcatcctgaagcagaacctgcagctggacagcttcagcacctaccgcgtgtacttcagcgtgagcggcgacgccaacgtgcgcatccgcaactcccgcgaggtgctgttcgagaagaggtacatgagcggcgccaaggacgtgagcgagatgttcaccaccaagttcgagaaggacaacttctacatcgagctgagccagggcaacaacctgtacggcggcccgatcgtgcacttctacgacgtgagcatcaagtag

SEQ ID NO: 225 - DNA, 2946 bp, synthetic construct
  source 1..2946: mol_type = other DNA
acaccaatgatgcaaataggctgggaatagtctgtctaatagtttgagtgaatcatgtcactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccatgaacaagaacaacaccaagctgagcacccgcgccctgccgagcttcatcgactacttcaacggcatctacggcttcgccaccggcatcaaggacatcatgaacatgatcttcaagaccgacaccggcggcgacctgaccctggacgagatcctgaagaaccagcagctgctgaacgacatcagcggcaagctggacggcgtgaacggcagcctgaacgacctgatcgcccagggcaacctgaacaccgagctgagcaaggagatccttaagatcgccaacgagcagaaccaggtgctgaacgacgtgaacaacaagctggacgccatcaacaccatgctgcgcgtgtacctgccgaagatcaccagcatgctgagcgacgtgattaagcagaactacgccctgagcctgcagatcgagtacctgagcaagcagctgcaggagatcagcgacaagctggacatcatcaacgtgaacgtcctgatcaacagcaccctgaccgagatcaccccggcctaccagcgcatcaagtacgtgaacgagaagttcgaagagctgaccttcgccaccgagaccagcagcaaggtgaagaaggacggcagcccggccgacatcctggacgagctgaccgagctgaccgagctggcgaagagcgtgaccaagaacgacgtggacggcttcgagttctacctgaacaccttc

SEQ ID NO: 226 - DNA, 209 bp, synthetic construct
  source 1..209: mol_type = other DNA
atgcaaataggctgggaatagtctgtctaatagtttgagtgaatcatgtcactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtac

SEQ ID NO: 227 - DNA, 20 bp, synthetic construct
  source 1..20: mol_type = other DNA
tgaatcatgtcactgatagt

SEQ ID NO: 228 - DNA, 20 bp, synthetic construct
  source 1..20: mol_type = other DNA
aaacgtccgccatggtctga

SEQ ID NO: 229 - DNA, 10579 bp, synthetic construct
  source 1..10579: mol_type = other DNA
tcctgtgttgttggaacagacttctgtctcttctggtgatcataaatatttaaatgaaccagttgtgttggaaaatgttgttttcttttgtctctagactggaaagcggagttctcgtcaacacggttctttcaactagggatgaaagtggtaatccgaattgttagtacaaatttaatattttaaaatagatatgtataaaattttatgttgatcttttttatgttatcaagcacattagtataaattagtataaatatgaataaaatattacataaaatgttttatgtattatttggtccctacaacataaatagttgaaaaaattactaaatttgttttcgaatctatatcgaagtttatatctattatttaagaaaaatataggatgaaaaggtttatcttttatgaatctttacaagctggatcttataaacaagaaaataaatttatattgtagattttatatcctatttattcgcaatcaaagaaaagcgactaaaaaactgattaccgagtaaatactgtttccaaccgttttcgtccctactatcaacgccttctcccaaccgcagtcgatctgtccgtctgtatcaggcgcagcggcacccctgctgttcgactatctagaccatagaatattttaggtatacaataattttagttccacgctagaacattttagttagaataataacaagatttgctattgatgtaggactcgcccgtcactgtctaaaaaagcattctgtcggtcttattctttaggcatcagcgggtgtactatctcatttttcctatcatattcctcagtactctgttaagtataaatggtctattttacatgatgaactaataaaactaattaaggatcctaactttttgtgaaggtaatttggatcattatgcattaccatcctacgtatacctgctgcagcagcatctgcgtaagcacagcctagatatatgcttctgtgtggactgaaaggagactttgtttatcaattagtatactcccaaaaaactgatgacaccaatgatgcaaataggctgggaatagtctgtctaatagtttgagtgaatcatgtcactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccatgaacaagaacaacaccaagctgagcacccgcgccctgccgagcttcatcgactacttcaacggcatctacggcttcgccaccggcatcaaggacatcatgaacatgatcttcaagaccgacaccggcggcgacctgaccctggacgagatcctgaagaaccagcagctgctgaacgacatcagcggcaagctggacggcgtgaacggcagcctgaacgacctgatcgcccagggcaacctgaacaccgagctgagcaaggagatccttaagatcgccaacgagcagaaccaggtgctgaacgacgtgaacaacaagctggacgccatcaacaccatgctgcgcgtgtacctgccgaagatcaccagcatgctgagcgacgtgattaagcagaactacgccctgagcctgcagatcgagtacctgagcaagcagctgcaggagatcagcgacaagctggacatcatcaacgtgaacgtcctgatcaacagcaccctgaccgagatcaccccggcctaccagcgcatcaagtacgtgaacgagaagttcgaagagctgaccttcgccaccgagaccagcagcaaggtgaagaaggacggcagcccggccgacatcctggacgagctgaccgagctgaccgagctggcgaagagcgtgaccaagaacgacgtggacggcttcgagttctacctgaacaccttccacgacgtgatggtgggcaacaacctgttcggccgcagcgccctgaagaccgccagcgagctgatcaccaaggagaacgtgaagaccagcggcagcgaggtgggcaacgtgtacaacttcctgatcgtgctgaccgccctgcaggcccaggccttcctgaccctgaccacctgtcgcaagctgctgggcctggccgacatcgactacaccagcatcatgaacgagcacttgaacaaggagaaggaggagttccgcgtgaacatcctgccgaccctgagcaacaccttcagcaacccgaactacgccaaggtgaagggcagcgacgaggacgccaagatgatcgtggaggctaagccgggccacgcgttgatcggcttcgagatcagcaacgacagcatcaccgtgctgaaggtgtacgaggccaagctgaagcagaactaccaggtggacaaggacagcttgagcgaggtgatctacggcgacatggacaagctgctgtgtccggaccagagcgagcaaatctactacaccaacaacatcgtgttcccgaacgagtacgtgatcaccaagatcgacttcaccaagaagatgaagaccctgcgctacgaggtgaccgccaacttctacgacagcagcaccggcgagatcgacctgaacaagaagaaggtggagagcagcgaggccgagtaccgcaccctgagcgcgaacgacgacggcgtctacatgccactgggcgtgatcagcgagaccttcctgaccccgatcaacggctttggcctgcaggccgacgagaacagccgcctgatcaccctgacctgtaagagctacctgcgcgagctgctgctagccaccgacctgagcaacaaggagaccaagctgatcgtgccaccgagcggcttcatcagcaacatcgtggagaacggcagcatcgaggaggacaacctggagccgtggaaggccaacaacaagaacgcctacgtcgaccacaccggcggcgtgaacggcaccaaggccctgtacgtgcacaaggacggcggcatcagccagttcatcggcgacaagctgaagccgaagaccgagtacgtgatccagtacaccgtgaagggcaagccatcgattcacctgaaggacgagaacaccggctacatccactacgaggacaccaacaacaacctggaggactaccagaccatcaacaagcgcttcaccaccggcaccgacctgaagggcgtgtacctgatcctgaagagccagaacggcgacgaggcctggggcgacaacttcatcatcctggagatcagcccgagcgagaagctgctgagcccggagctgatcaacaccaacaactggaccagcaccggcagcaccaacatcagcggcaacaccctgaccctgtaccagggcggccgcggcatcctgaagcagaacctgcagctggacagcttcagcacctaccgcgtgtacttcagcgtgagcggcgacgccaacgtgcgcatccgcaactcccgcgaggtgctgttcgagaagaggtacatgagcggcgccaaggacgtgagcgagatgttcaccaccaagttcgagaaggacaacttctacatcgagctgagccagggcaacaacctgtacggcggcccgatcgtgcacttctacgacgtgagcatcaagtaggagctctagatctgttctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccaggggtaccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccccgggtctagacaattcagtacattaaaaacgtccgccatggtctgaaggcaacagataaggcatactgggccttgtggtagttgttttactgggcctttttgtatgatctataaaattcactgggatcaacccggagaggaatggcagcagatgcagtccccagggtcctccgtcgccgcctgagcacccggcacccgcgctgaaccggagagggacgcgcggacgccgtgcagctggtgcggagggggctgtggcagatgaggatgagacgcgtacgtggctgggaaggccagcaggccaccgggtcttcgtccagcccggcgcgagtggacaggactagagatggcaacggttacaaacccgctgggttttaccgtcccaaacccgtacccgtgaaaaatatctatgcccattaaaaaacccgtacccatgacgggtttgagattttgcccaaacccgtacccatcgggttaacgggtacccatgggttacccgcgggtttcatctccaatatacctgttcttctcataatcaataagtatcgtaatgattaatgatatcatgatccaaaatctatgtaatgaacaacgagttcatgatttggtataaaaattattagtagagagaatgaaatacaaataataagttgtataattaagtgaccttgcactaagttatccatccatcacatatataacgctagtaaaaactataatatcaagcaagcaacactctcaccgactactgatacattcaccaattgataaaaaatatgaagtaaataaggaataacaagtttgttgttcgtttataaaataaaatgacaatatgcactaggtttggtcgggtttaaaaaacccacgggttcacgggtttgggtactataggaacaaacccgtacccataaacccattgggtacagatttatgcccgttaacaaacccatgggtatgaaaattgacccaaacctataccctaatggggtaaaaacccatcgggtttcggatttcgggtacccattgccatctctagacaggacaacctcggccggtcctgtatgtaggccaccagcatcggccagttggtacatccagccggggtcaggtcacttttactcgtctcaatcagacaatcaccgtccaccaacgaacgccaacgttgtcacttgtcaggtcggttgagacttgtatttttttttgtcctccgtaaaaatcggttcaccag

SEQ ID NO: 230 - DNA, 1230 bp, synthetic construct
  source 1..1230: mol_type = other DNA
cctgtgttgttggaacagacttctgtctcttctggtgatcataaatatttaaatgaaccagttgtgttggaaaatgttgttttcttttgtctctagactggaaagcggagttctcgtcaacacggttctttcaactagggatgaaagtggtaatccgaattgttagtacaaatttaatattttaaaatagatatgtataaaattttatgttgatcttttttatgttatcaagcacattagtataaattagtataaatatgaataaaatattacataaaatgttttatgtattatttggtccctacaacataaatagttgaaaaaattactaaatttgttttcgaatctatatcgaagtttatatctattatttaagaaaaatataggatgaaaaggtttatcttttatgaatctttacaagctggatcttataaacaagaaaataaatttatattgtagattttatatcctatttattcgcaatcaaagaaaagcgactaaaaaactgattaccgagtaaatactgtttccaaccgttttcgtccctactatcaacgccttctcccaaccgcagtcgatctgtccgtctgtatcaggcgcagcggcacccctgctgttcgactatctagaccatagaatattttaggtatacaataattttagttccacgctagaacattttagttagaataataacaagatttgctattgatgtaggactcgcccgtcactgtctaaaaaagcattctgtcggtcttattctttaggcatcagcgggtgtactatctcatttttcctatcatattcctcagtactctgttaagtataaatggtctattttacatgatgaactaataaaactaattaaggatcctaactttttgtgaaggtaatttggatcattatgcattaccatcctacgtatacctgctgcagcagcatctgcgtaagcacagcctagatatatgcttctgtgtggactgaaaggagactttgtttatcaattagtatactcccaaaaaactgatgacaccaatgatgcaaataggctgggaatagtctgtctaatagtttgagtgaatcatgtcactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagcc

SEQ ID NO: 231 - DNA, 1518 bp, synthetic construct
  source 1..1518: mol_type = other DNA
tctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccccgggtctagacaattcagtacattaaaaacgtccgccatggtctgaaggcaacagataaggcatactgggccttgtggtagttgttttactgggcctttttgtatgatctataaaattcactgggatcaacccggagaggaatggcagcagatgcagtccccagggtcctccgtcgccgcctgagcacccggcacccgcgctgaaccggagagggacgcgcggacgccgtgcagctggtgcggagggggctgtggcagatgaggatgagacgcgtacgtggctgggaaggccagcaggccaccgggtcttcgtccagcccggcgcgagtggacaggactagagatggcaacggttacaaacccgctgggttttaccgtcccaaacccgtacccgtgaaaaatatctatgcccattaaaaaacccgtacccatgacgggtttgagattttgcccaaacccgtacccatcgggttaacgggtacccatgggttacccgcgggtttcatctccaatatacctgttcttctcataatcaataagtatcgtaatgattaatgatatcatgatccaaaatctatgtaatgaacaacgagttcatgatttggtataaaaattattagtagagagaatgaaatacaaataataagttgtataattaagtgaccttgcactaagttatccatccatcacatatataacgctagtaaaaactataatatcaagcaagcaacactctcaccgactactgatacattcaccaattgataaaaaatatgaagtaaataaggaataacaagtttgttgttcgtttataaaataaaatgacaatatgcactaggtttggtcgggtttaaaaaacccacgggttcacgggtttgggtactataggaacaaacccgtacccataaacccattgggtacagatttatgcccgttaacaaacccatgggtatgaaaattgacccaaacctataccctaatggggtaaaaacccatcgggtttcggatttcgggtacccattgccatctctagacaggacaacctcggccggtcctgtatgtaggccaccagcatcggccagttggtacatccagccggggtcaggtcacttttactcgtctcaatcagacaatcaccgtccaccaacgaacgccaacgttgtcacttgtcaggtcggttgagacttgtatttttttttgtcctccgtaaaaatcggttcaccag

SEQ ID NO: 232 - DNA, 2401 bp, synthetic construct
  source 1..2401: mol_type = other DNA
ctaacacttagacaagatgttagtacacaaaactaatgtactaaggcttagaaacatacctttacttgtgatttacactttgttcatccatgagcatattttcacatttaggcccttgtgtttgcactcaatcaccaaaatacttagaaatggcccaagggcacatttccctttcaatctccccctttttggtgatttatgccaacacaacataaagcaagtggaacaagtgcaaaatcacttcaaataaaaactcaaattggttttgattcaattttggcatatatggatcatcctttgccaccacttggtttgtttttgcaaatcaaactcaaatctctatctataagtcaaacacacatattgaagcataaagagagtcatttcaaaagaatttgaccacggatttcaaaaactccccatttttcccataatcaacacttctccccacaagaagccaacttttgaaaatagagacaataagagacaataaaagcttttgacaaaacaaaaactctattctactattttcaaaatctctcaagtggtagctgatccatttatcactttggcctttattttctccccctttggcatcaagcaccaaaacgggatcaatcttggcctgttaaccccattgcctcaccaaagtcttcaattaagagcaaatggcaataagatttcatgagatgaacttggaattagttaccctctcatcggagtgcagtggaagtctttcatggtccaagtccaccttttccctttcaatcctccttcgagactaaattatcaaactcaagcacatggttagtctcaaagggtcaagttgtaacacatctccccctacacatgtgcatcactttgcaacggacttgtgaggtccagggagtgtttgtacaacttgagcaccataataagcaacataatgcaaaaaggaacatgatcaaaagcataactacatgtatgctacaattcaatccaggttccgcgaatctaagacatttagctcactacgcaacctgcaaaaggtcttctcatctagaggcttagtgaagatatcggctagctggttctcggtgctaacatgaaacacttcgatatctcccttttgctggtggtctctcaaaaagtgatgccggatgtctatgtgctttgtgcggctgtgttcaacaggattttccgccatgcggatagcactctcattatcacataggagtgggactttgctcagattgtagccaaagtccctgagggtttgcctcatccaaagtagttgcgcgcaacactgtcctgcggcaacatactcggcctcagcggtggatagggcaacggaagtttgtttcttagagttccatgacaccagggaccttcctaagaattggaacgtccccgatgtactcttcctatcgaccttacatccagcatagtcggagtctgagtatccaactaagtcaaaggtagacccctttggataccagagcccgaagcaaggcgtagcaaccaaatatctaagaattcgcttcaccgccactaagtgacactccttaggatcggattgaaatctagcacacatgcatacgctaagcataatatccggtctactagcacataaataaagcaaagaacctatcattgaccggtatgctttttgatcaacggacttacctcctttgttgaggtcggtgtgtccgtcggtccccatcggagtctttgcgggcttggcgtccttcatcccaaactgctttagcagatcttgcgtgtacttcgtttgggagatgaaggtgccgtccttgagttgcttcacttggaacccaaggaagtagttcaactcgcctatcatcgacatctcgaatttctgcgtcatcaccctgctaaactcttcacaagacttttggttagtagaaccaaatattatgtcatcgacataaatttggcacacaaacaaatcaccatcacatgtctttgtaaaaagagttggatcggccttcccaaccttgaaagcattagcaagtaaaaagtctctaaggcattcataccatgctcttggggcttgcttaagtccatagagcgccttagagagcttacacacatggtcggggtaccgttcatcctcgaagccagggggttgctctacgtacacctcctccttgattggcccgttgaggaaagcgctcttcacatccatttggaacaacctgaaagaatggtgagcggcatatgctagcaagattcgaattgactctagcctagccacaggagcaaaagtctcctcaaaatccaaacctgcgacttgggcataaccttttgccacaagtcgagccttgtttctcgtcaccaccccgtgctcgtcctgtttgttgcggaacacccacttggttcccacaacattttgctttggacg

SEQ ID NO: 233 - DNA, 2404 bp, synthetic construct
  source 1..2404: mol_type = other DNA
agcacttagacaaaatatgttagtactgaaaacaatgtactaagtatagaaacatacctcatgtcttgatttgcacttttctcttcatttagcacataagaactcaattaaatgtgttgggtacttaatcaccaaaacataatagaaattgcccaagggcacatttccctttcaatctccccctttttggtgatttatgccaacacatccaaaagcaaccaaaagaagtgcaacatcaatgcaattgagaacaaaatggtctttgcacaagttttgacatatttggatcattctttgccaccacttggtttgtttttgcaaatcaaattcattttcctatctctaagtcaaacacacttgtttaggcacaaagagagatattccaatggaaaaattgatcaagtaccaaaaactcccccttttgccataataaaaaattctccccccataagaggccaaattttgcaataagtgtattttggacaaatcaaaaattctaactctactattttcaaaattctcaagtggtagctgatccatttgatttggccttaatttctccccctttggcattaagcaccaaaacgggatcactcttggccctttaaccccattgcctcaccaaaaatgtcaataaagagcaaaaaggcaactagagcataagaatgaacttggaggtgagtacactaataccggagtgcagtggaagtctttgcgtggtccaagttcacctttcccttccaattcacctttgagactacatcaagtatactcaaagacaagggttagtctcaaagggtcaagttgtagcagttctccccctaaatatgtgcatcattcacacatggacttgtgaggtctggggattccttgcacaacttgaacaccataaataagcaacaaattacataaatgcataagtaacatgatcaaaggcatagaacacatgtatgttatagatcaatccaagttatgcgaatctaagacatttagctcagtacgtaacctgcaaaaggttttctcatccaacggcttggtaaagatatcggctagctagttctcggtgctaatatggaacacctcgatatctcccttttgctagtggtctctcagaaagtgatgccggatgtctatgtgcttagtgcggctgtgttcaacaggattatctgccatgtggattgcactctcattgtcacataggagtgggactttgctcagattgtagccaaagtcccggagggtttgcctcatccaaagtagttgcgcgcaacactgtcctgcggcaacatactcggcctcagcggtggatagggcaacggaggtttgtttctttaagctccaagacaccagggaccttcctaggaattggcacgtccatgatgtactcttcctatcaaccttgcatccagcataatgggagtctgaatatccaatcaagtcaaaggtaaaccccttaggataccagatcccgaagcaaggcgtagaaactaaatatctaagaatccgcttaacggccacaaggtgacattcccttgggtcggattgatatctagcacacatgcatacgctaagcataatatctggtctactagcacataaataaagtaatgaccctatcattgaccgatatgccttttgatcaacggacttacctcctttgttgaggtcgacatgcccgtcggttcccataggtgtcttcgcgggcttggcgtccttcatcccaaaccgcttgagaagatcttgagtgtacttcgtttgggagaagaaggtgccgtccttgagttgcttcacttggaacctgtcggggaccacaattaggggtaccctcaagacgcctaattctcagatggtaaccccatcagcataaagctgcagaggcctgatgggtgcgattaagtcagggatcagtccatacgagtgactcgatcacgcttcacccgagcctagcctcggactcgggcagccgacctcgagggacttctgtctcgcccgaggccccccttttaacggcggacacatctccggctcgcccgaggccttggcttcgctaagaagcaaccctgactaaatcgccgcaccgactgaccaagttgcaggagcatttaacgcaaaggtggcctgacacctctatcctgacgcgcgccccccggcagagccgaagtgaccgccgtcactccgccgctccactgaccggtctgacagaaggacagcgccgcctgcgccactccgactgcagtgccactcgacagagtaagtctgacaggcagtcagacctcgccagaggcgccataggaaactccgctccgcccgaccccagggctcggactcgggctaagacccagaagacggcgaactccgctccgcccgaccctagggctcggactcgggctaagacccggaagac

SEQ ID NO: 234 - DNA, 14405 bp, synthetic construct
  source 1..14405: mol_type = other DNA
atgaacaagaacaacaccaagctgagcacccgcgccctgccgagcttcatcgactacttcaacggcatctacggcttcgccaccggcatcaaggacatcatgaacatgatcttcaagaccgacaccggcggcgacctgaccctggacgagatcctgaagaaccagcagctgctgaacgacatcagcggcaagctggacggcgtgaacggcagcctgaacgacctgatcgcccagggcaacctgaacaccgagctgagcaaggagatccttaagatcgccaacgagcagaaccaggtgctgaacgacgtgaacaacaagctggacgccatcaacaccatgctgcgcgtgtacctgccgaagatcaccagcatgctgagcgacgtgatgaagcagaactacgccctgagcctgcagatcgagtacctgagcaagcagctgcaggagatcagcgacaagctggacatcatcaacgtgaacgtcctgatcaacagcaccctgaccgagatcaccccggcctaccagcgcatcaagtacgtgaacgagaagttcgaagagctgaccttcgccaccgagaccagcagcaaggtgaagaaggacggcagcccggccgacatcctggacgagctgaccgagctgaccgagctggcgaagagcgtgaccaagaacgacgtggacggcttcgagttctacctgaacaccttccacgacgtgatggtgggcaacaacctgttcggccgcagcgccctgaagaccgccagcgagctgatcaccaaggagaacgtgaagaccagcggcagcgaggtgggcaacgtgtacaacttcctgatcgtgctgaccgccctgcaggcccaggccttcctgaccctgaccacctgtcgcaagctgctgggcctggccgacatcgactacaccagcatcatgaacgagcacttgaacaaggagaaggaggagttccgcgtgaacatcctgccgaccctgagcaacaccttcagcaacccgaactacgccaaggtgaagggcagcgacgaggacgccaagatgatcgtggaggctaagccgggccacgcgttgatcggcttcgagatcagcaacgacagcatcaccgtgctgaaggtgtacgaggccaagctgaagcagaactaccaggtggacaaggacagcttgagcgaggtgatctacggcgacatggacaagctgctgtgtccggaccagagcgagcaaatctactacaccaacaacatcgtgttcccgaacgagtacgtgatcaccaagatcgacttcaccaagaagatgaagaccctgcgctacgaggtgaccgccaacttctacgacagcagcaccggcgagatcgacctgaacaagaagaaggtggagagcagcgaggccgagtaccgcaccctgagcgcgaacgacgacggcgtctacatgccactgggcgtgatcagcgagaccttcctgaccccgatcaacggctttggcctgcaggccgacgagaacagccgcctgatcaccctgacctgtaagagctacctgcgcgagctgctgctagccaccgacctgagcaacaaggagaccaagctgatcgtgccaccgagcggcttcatcagcaacatcgtggagaacggcagcatcgaggaggacaacctggagccgtggaaggccaacaacaagaacgcctacgtggaccacaccggcggcgtgaacggcaccaaggccctgtacgtgcacaaggacggcggcatcagccagttcatcggcgacaagctgaagccgaagaccgagtacgtgatccagtacaccgtgaagggcaagccatcgattcacctgaaggacgagaacaccggctacatccactacgaggacaccaacaacaacctggaggactaccagaccatcaacaagcgcttcaccaccggcaccgacctgaagggcgtgtacctgatcctgaagagccagaacggcgacgaggcctggggcgacaacttcatcatcctggagatcagcccgagcgagaagctgctgagcccggagctgatcaacaccaacaactggaccagcaccggcagcaccaacatcagcggcaacaccctgaccctgtaccagggcggccgcggcatcctgaagcagaacctgcagctggacagcttcagcacctaccgcgtgtacttcagcgtgagcggcgacgccaacgtgcgcatccgcaactcccgcgaggtgctgttcgagaagaggtacatgagcggcgccaaggacgtgagcgagatgttcaccaccaagttcgagaaggacaacttctacatcgagctgagccagggcaacaacctgtacggcggcccgatcgtgcacttctacgacgtgagcatcaagtaggagctctagatctgttctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccaggggtaccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccccgggtctagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccacc

SEQ ID NO: 235 - DNA, 17 bp, synthetic construct
  source 1..17: mol_type = other DNA
atgcttttcgagcatgc

SEQ ID NO: 236 - DNA, 19 bp, synthetic construct
  source 1..19: mol_type = other DNA
catatgcttttcgggcatg

SEQ ID NO: 237 - DNA, 19 bp, synthetic construct
  source 1..19: mol_type = other DNA
tgtccgcttcttggctcta

SEQ ID NO: 238 - DNA, 23 bp, synthetic construct
  source 1..23: mol_type = other DNA
actgggccggtcatcattcttct

SEQ ID NO: 239 - DNA, 17 bp, synthetic construct
  source 1..17: mol_type = other DNA
actaggccaaccaagcc

SEQ ID NO: 240 - DNA, 16 bp, synthetic construct
  source 1..16: mol_type = other DNA
actaggccacccaagc

SEQ ID NO: 241 - DNA, 19 bp, synthetic construct
  source 1..19: mol_type = other DNA
tcacatgggccacgttttc

SEQ ID NO: 242 - DNA, 19 bp, synthetic construct
  source 1..19: mol_type = other DNA
gttgggctagcgtgggaat

SEQ ID NO: 243 - DNA, 24 bp, synthetic construct
  source 1..24: mol_type = other DNA
tgcctaataatttcttatatttct

SEQ ID NO: 244 - DNA, 25 bp, synthetic construct
  source 1..25: mol_type = other DNA
atgtgcctaataatttcttgtattt

SEQ ID NO: 245 - DNA, 25 bp, synthetic construct
  source 1..25: mol_type = other DNA
aattttctgtaaaagttgagtatca

SEQ ID NO: 246 - DNA, 18 bp, synthetic construct
  source 1..18: mol_type = other DNA
gcgcagaatgggaatgtc

SEQ ID NO: 247 - DNA, 18 bp, synthetic construct
  source 1..18: mol_type = other DNA
tagagcttaccgactgta

SEQ ID NO: 248 - DNA, 18 bp, synthetic construct
  source 1..18: mol_type = other DNA
tagagcttactgactgta

SEQ ID NO: 249 - DNA, 20 bp, synthetic construct
  source 1..20: mol_type = other DNA
cgtaggcttcccgcactatg

SEQ ID NO: 250 - DNA, 20 bp, synthetic construct
  source 1..20: mol_type = other DNA
aggctttgttgtggcctggt

SEQ ID NO: 251 - DNA, 15 bp, synthetic construct
  source 1..15: mol_type = other DNA
tccacaccaacctga

SEQ ID NO: 252 - DNA, 15 bp, synthetic construct
  source 1..15: mol_type = other DNA
cacaccagcctgaac

SEQ ID NO: 253 - DNA, 18 bp, synthetic construct
  source 1..18: mol_type = other DNA
gcaccgcatggtctccac

SEQ ID NO: 254 - DNA, 20 bp, synthetic construct
  source 1..20: mol_type = other DNA
gagggcttccacaacctgat

SEQ ID NO: 255 - DNA, 642 bp, synthetic construct
  source 1..642: mol_type = other DNA
tctgtaaaacaaacagccatttgatccattcagcctgatttttcgtatagggtcaaaaaacttggtaacttcaaagcaaggcactaaatgttcacatgaacaaaacaaagggcagacccagctcccacatgaatgtggggtctttcccccacaaatgcggaaaggaaaatccagcatgtaaattcatccaagcaacagaaacaaaaactcggccngggaaggaaagaccgccctcccggtattctattaagaagagaccgaaacatggtcccggccgaaaaaatccccgaaccctagccccccatcactagttggccgacatcgcgcactctgcaaatgcccagccggagggtggggtgcatgacataacccgagggcgggcggggcacaacgaagggattttttaaccaagcccgaaattcgcccccaagggggatcgaacccgggacctggaggtgctacttggaagctttaaccattacgctaaaggccctttcgcagcgacagaaacacacataagttgattcctaaactaaagctgttacagaaaaacagtaatgtccgaactccggatatgtagattatctaacagaactgatcaccagaagcaaagggcctgttcagtttgggtttcagctgctg